(19) United States
(12) Patent Application Publication
(10) Pub. No.: US 2017/0224520 A1
(43) Pub. Date: Aug. 10, 2017

(54) METHODS FOR INTEGRATING SENSORS AND EFFECTORS IN CUSTOM THREE-DIMENSIONAL ORTHOSIS

(71) Applicant: Osteoid Saglik Teknolojileri A.S., Istanbul (TR)

(72) Inventor: Deniz Karasahin, Izmir (TR)

(73) Assignee: Osteoid Saglik Teknolojileri A.S., Istanbul (TR)

(21) Appl. No.: 15/496,997

(22) Filed: Apr. 25, 2017

Related U.S. Application Data
(63) Continuation of application No. PCT/IB2015/002432, filed on Nov. 2, 2015.
(60) Provisional application No. 62/075,082, filed on Nov. 4, 2014.

(51) Int. Cl. A61F 5/02; G06K 9/00; A61B 5/11; A61F 5/058; A61F 5/01

(52) U.S. Cl. CPC A61F 5/022; A61F 5/05841; A61F 5/01; A61B 5/1116; A61B 5/1121; A61B 5/11; G06K 9/00201; A61F 2005/0167; A61F 2005/0197; G06K 2209/40

(57) ABSTRACT

A conformable body interface includes a body scaffold comprising a three-dimensional lattice which can be removably placed over a three-dimensional soft-tissue surface, such as a knee, elbow, spine, ankle, wrist, hip, or neck. One or more sensors are located at one or more locations on the body scaffold, and the one or more locations are selected to position the sensor near a target region on the body surface when the body scaffold is placed over the three-dimensional body surface. Typically, the sensors are positioned near a body joint to detect motion of the body joint.

### CROSS-REFERENCE TO RELATED APPLICATION

This application is a continuation of PCT application no. PCT/IB2015/002432 (Attorney Docket No. 50016-703.601) filed Nov. 2, 2015, which claims priority from provisional application 62/075,082 (Attorney Docket No. 50016-703.101), filed on Nov. 4, 2014, the full disclosures of which are incorporated herein by reference.

### BACKGROUND OF THE INVENTION

1. Field of the Invention

While orthotic interventions for physical rehabilitation have been known for centuries, they still present challenges in implementation. Starting with temporary immobilization using splints made from sticks, the field has progressed to the use of space age composites for fabricating sophisticated modern dynamic orthosis. The latest step in this evolutionary path is the use of three-dimensional imaging and printing for fabricating personalized orthosis, custom build for individual patient anatomies.

In contemporary casting and splinting applications, the injured area is isolated from its surrounding environment. As a result of this isolation many beneficial technologies assisting the rehabilitation process cannot be used while the injured area remains splinted. Fundamental problem in current monitoring and medical rehabilitation efforts is the inability to repeatedly and precisely position sensors/effectors at a particular anatomical location to allow consistent data mining/delivery of therapeutic stimulation.

The ability to model and fabricate splints and other body scaffolds from three-dimensional image scans of a patient have opened up a new field of orthopedic treatment and monitoring. It would be desirable to utilize these new technologies to enable a treatment and monitoring protocols that were njot previously available. In particular, it would be useful to provide diagnostic systems which allow integrated healthcare solutions for the orthopedics industry

2. Description of the Background Art

Relevant background patents and publications include U.S. Pat. Nos. 5,107,854; 5,823,975; 5,836,902; 6,179,800; 6,725,118; 7,632,216; 8,613,716; US2003/0032906; US2007/0132722; US2009/146142; US2011/0004074; US2011/0301520; US2011/0302694; and EP 2671544. The design and fabrication of body splints and casts incorporating sensors and treatment element are described in US2014267116; WO2015/124900; WO2015/032006; WO2007/056734 and U.S. Pat. No. 8,838,263B2.

### BRIEF SUMMARY OF THE INVENTION

The present invention can combine computer-aided design, software analytics, digital manufacturing, sensory, medically beneficiary and digital data collection and analysis technologies to create a digital process for manufacturing, personal three-dimensional printed orthoses with monitoring and compliance capabilities. The present invention may incorporate diagnostic capabilities and/or therapeutic capabilities. In particular, one invention is directed at methods and apparatus for determining the motion of a body joint which is useful in monitoring joint stability in a variety of therapeutic and rehabilitative situations. Other inventions are also described herein.

Physical examinations are essential for diagnosis and classification of numerous neuromuscular and musculoskeletal systems. The diagnostic systems of the resent invention can digitize physical examination processes by providing anthropometric sensor placement patterns. The custom, conformable medical devices of the present invention are further useful to collect, store, and analyze biomechanical data obtained by the sensors, commonly referred to as “data mining” Different sets of sensory input in this digital environment enable both personal, population-based data analytics for specific diagnostic challenges.

The present invention also provides therapeutic systems which converts personal three-dimensional printed orthotics and enable the manufacturing of custom conformable medical devices for relevant therapeutic and medical beneficiary delivery challenges. The therapeutic system specifically, provides a reliable platform for medical purposes.

The sensor and therapeutic systems of the present invention may incorporate any custom, three-dimensional printed or molded body scaffold, such as an orthotic device, configured for static or dynamic orthotic intervention and rehabilitation. Exemplary three-dimensional orthoses include upper-limb orthoses, lower-limb orthoses, spinal and neck orthoses, wrist orthoses, ankle orthoses, and similar applications. Monitoring technologies include sensors of any kind (compliance sensors, inertial measurement units, tilt sensor, stretch sensor, pressure/tension sensors, accelerometers, gyroscopes, magnetometers, velocity sensors, pulse sensors, pulse oximeter, electromyography sensors, sensory ultrasound, electrical impedance tomography, etc.). A target body surface will typically be scanned directly or indirectly (from a mold) to generate a three-dimension data set representing the body surface geometry. Three-dimensional design software (CAD) may be used to both design the body scaffold and to place the sensor or therapeutic elements on the scaffold, typically by locating a receptacle or other attachment point on the scaffold. Design software may employ any one or more of a variety of design protocols, such as finite element analysis, generative design, and virtual and augmented reality technologies. The scaffolds may also have other capabilities including internet-of-things (IOT) devices and systems, signal processing units, wireless communication units (Bluetooth®, infrared, GSM, local wireless networks, and internet connectivities), on-board or remote interfaces (tactile, photometric, augmented or virtual reality interface, web or software interface linked mobile devices, smart phones, LED, LCD, tablets, laptops etc.), various types of power sources (including thermoelectric generation, wireless energy transfer technologies, alternative and direct current), cloud computing and storage units. Certain medically beneficial effectors include devices which deliver therapeutic stimulation (electromyography, ultrasound stimulation, low-intensity pulsed ultrasound (LIPUS), TENS, EMS, cryotherapy, and thermotherapy etc.) and pharmaceutical administration (dermal administration, injection, electroporation, etc.).

In a first specific aspect, the present invention provides a method for fabricating a conformable body interface which can sense motion of a body joint. A body scaffold which can be removably placed over a three-dimensional body surface is fabricated to conform to one or more target regions of said body surface adjacent to the body joint. At least one sensor element is attached to the body scaffold at a location selected to position the sensor near the target region on the body surface when the body scaffold is placed over the three-dimensional body surface. The sensor is configured to detect motion of the body joint, typically by measuring or sensing at least one of pressure or tension. Typically, two, three, or more individually sensors will be placed at selected location about the joint so that a variety of body motions can be tracked in real time, such as flexion, extension, rotation, pronation, and supination. Sensors may be selected from the group consisting of pressure sensors, strain sensors, force sensors, accelerometers, gyroscopes, velocity sensors, tilt sensors and pulse sensors.

While the body scaffold can be fabricated by conventional techniques, including direct casting from the body surface, the present invention is particularly useful when incorporating digital scanning and design techniques as described previously. Thus, a preferred fabrication technique will first obtain a data set representing the three-dimensional, soft tissue body surface adjacent to a body joint. The data set may be obtained by scanning the three-dimensional soft-tissue body surface of a patient to produce an initial data set representing the geometry of the one or more target regions on the soft tissue body surface. The scanning may be direct optical scanning of the actual body surface or may be indirect scanning of a mold or other representation taken of the body surface. The data set is then modified to include locations for attaching the one or more sensors to the body scaffold. Other features as noted above may also be designed into the scaffold during the design phase.

The body scaffold will usually be fabricated from the design data set by three-dimensional printing, also referred to as stereo lithography, but other digital fabrication methods, such as numerically controlled machining of a substrate, may also be employed. Fabrication will further include attaching the sensors or other interface elements to the body scaffold, typically by inserting sensor elements into receptacles that are defined in the data set and/or by securing the sensors or interface elements to marked locations on the body scaffold that are defined in the initial data set.

In a second specific aspect, the present invention provides a method for generating a data set for fabricating a conformable body scaffold. A three-dimensional soft-tissue body surface of a patient is directly or indirectly scanned to produce an initial data set representing the surface geometry of at least one target region on the soft tissue body surface adjacent to a body joint. The initial data set is then modified to include one or more locations for attaching one or more sensors configured to detect motion of the body joint to the conformable body scaffold to produce a final data set suitable for controlling a fabrication machine to produce the conformable body scaffold. The initial data set typically defines a lattice structure which at least partially circumscribes the soft tissue surface, and the soft tissue surface may be any one of an upper limb, a lower limb, a wrist, an ankle, a spine, a neck or the like.

In a third specific aspect, the present invention provides a conformable body interface including a body scaffold and one or more sensors. The body scaffold is typically formed as a three-dimensional lattice which can be removably placed over a three-dimensional soft-tissue surface. The one or more sensors are attached to one or more locations on the body scaffold, wherein the one or more locations are selected to position the sensors near the target region on the body surface when the body scaffold is placed over the three-dimensional body surface, wherein at least some of the sensors are configured to detect motion of the body joint.

The sensors are typically configured to detect at least one of flexion, extension, rotation, pronation, and supination, and the sensors may be selected from the group consisting of pressure sensors, strain sensors, force sensors, accelerometers, gyroscopes, velocity sensors, tilt sensors and pulse sensors. The body scaffold is often formed as an orthotic aid. In some embodiments, the conformable interface element may further include a therapeutic element, e.g. an ultrasound transducer, a heat source, a cooling source, an electrical source for muscle stimulation, an electrical source for electroconvulsive therapy, or a magnetic source.

In further aspects, the present invention provides a method for fabricating a comfortable body interface which can sense biomechanical forces on a body portion. A body scaffold which can be removable placed over a three-dimensional body surface is fabricated to conform to one or more target regions of said body surface adjacent to the patient anatomy. At least one sensor element is attached to the body scaffold at a location selected to position the sensor near the target region on the body surface when the body scaffold is placed over the three-dimensional body surface. The sensor is configured to detect pressure and tension on the patient anatomy. Typically, one sensor with pressure or tension monitoring capability is enough to detect biomechanical forces caused by edema accumulation and swelling. Also two, three, or more individually sensors can be placed at selected location about the anatomy to provide even deeper analysis in post fracture and diabetes cases. Sensors may be selected from the group consisting of pressure sensors, strain sensors, force sensors, pulse sensors, temperature sensors, oximeters etc.

In still further aspect, the present invention provides a method for fabricating a comfortable, conformable body interface and an activator unit (diagnostic probe) which can sense the kinematics of anatomic motion during orthopedic evaluation. A diagnostic probe which can be removably placed over a three-dimensional body surface is fabricated to conform to one or more target regions of said body surface adjacent to the patient anatomy. At least two sensor elements are attached to the diagnostic probe which attaches to pre-determined locations on body scaffold. The sensors are configured to detect pressure and changes in 3d space due to anatomic motion. Typically, one sensor with pressure monitoring capability and one sensor with gyroscopes and accelerometers is sufficient. Also, three, or more individually sensors can be placed at selected location about the anatomy to provide even deeper analysis in a variety of anatomic motion in different anatomic planes can be tracked in real time, such as flexion, extension, rotation, pronation, and supination. Sensors may be selected from the group consisting of pressure sensors, strain sensors, force sensors, pulse sensors, temperature sensors, oximeters, accelerometers, gyroscopes, velocity sensors, tilt sensors, and the like.

In yet further aspects, the present invention provide a method for applying adjustable external pressure units attachable to comfortable body interfaces. An external mechanical unit attachable to a body scaffold which can be removably placed over a three-dimensional body surface is fabricated to conform one or more target regions of said body surface adjunct to the anatomy. At least one sensor element is embedded to the adjustable pressure delivering unit at a location selected to deliver pressure on the targeted region. The sensors are configured to detect the adjustable pressure. Typically two, three or more individual external pressure units may be used to apply precision progressive orthosis.

In additional aspects, the present invention provides a method for fabricating a comfortable body interface as a hub for therapeutic equipment. In some embodiments, the conformable interface element may further include a therapeutic element, with an external electrical power source. A body scaffold which can be removably placed over a three-dimensional body surface is fabricated to conform to one or more target regions of said body surface adjacent to the anatomy. The orthotic is configured to stabilize certain medically beneficial therapy delivering probes to enable their usage during orthotic intervention periods, for LIPUS, TENS, EMS etc.

In more aspect, the present invention provides a method for fabrication a comfortable body interface as a part of a thermotherapy or cryotherapy delivering device with an external circulation pump and tubing. In some embodiments, the comfortable interface element may incorporate structural compartments for allowing circulation of hot or cold liquid to deliver thermotherapy or cryotherapy to conform to one or more target regions of said body surface adjacent to the anatomy.

In yet more aspects, the present invention provides a method for fabrication a comfortable body interface to house a pharmaceutical administration device. The pharmaceutical administration device may incorporate techniques and equipment for dermal administration, injection, electroporation.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an apparatus which assists in placing an arm in desired scanning position.

FIG. 2 illustrates the placement device of FIG. 1 in an imaging field.

FIG. 3 defines the carpal region (wrist) in the upper extremity with carpal bones and neighboring skeletal system elements.

FIG. 4 illustrates the areas on the wrist with the highest pressure profile caused by flexion and extension attempts.

FIG. 5 illustrates the areas on the wrist with the highest pressure profile caused by ulnar and radial deviation attempts.

FIG. 6 illustrates the area on the wrist with the highest pressure profile caused by pronation attempt.

FIG. 7 illustrates the area on the wrist with the highest pressure profile caused by supination attempt.

FIG. 8 defines the elbow region of the upper extremity with neighboring skeletal system elements.

FIG. 9 illustrates the areas on the elbow with the highest pressure profile caused by flexion and radial deviation attempts.

FIG. 10 defines the tarsal (ankle) region in the lower extremity with tarsal bones and neighboring skeletal system elements.

FIG. 11 illustrates the areas on the ankle with the highest pressure profile caused by dorsiflexion and plantarflexion attempts.

FIG. 12 illustrates the areas on the ankle with the highest pressure profile caused by inversion and eversion attempts.

FIG. 13 illustrates the areas on the ankle with the highest pressure profile caused by supination attempt.

FIG. 14 illustrates the areas on the ankle with the highest pressure profile caused by pronation attempt.

FIG. 15 defines the metatarsal and phalanges in the lower extremity with neighboring skeletal system elements.

FIG. 16 illustrates the areas on the toes with the highest pressure profile caused by claw toe deformity.

FIG. 17 defines the knee region in the lower extremity with patella and neighboring skeletal system elements.

FIG. 18 illustrates the areas on the knee with the highest pressure profile caused by flexion and extension attempts.

FIG. 19 defines the spine region in the torso with cervical, thoracic and lumbar spine regions.

FIG. 20 illustrates the areas on the cervical, thoracic and lumbar spine regions with the highest pressure profile caused by flexion in such areas of the spine.

FIG. 21 illustrates the areas on the cervical, thoracic and lumbar spine regions with the highest pressure profile caused by extension to left and right in such areas of the spine.

FIG. 22 defines flexor and extensor muscles in the forearm.

FIG. 23 illustrates the proportion of the forearm where post fracture swelling is the highest.

FIG. 24 fines flexor and extensor muscles in the upper arm.

FIG. 25 illustrates the proportion of the arm where the post fracture swelling is the highest.

FIG. 26 defines compartments of the lower leg.

FIG. 27 illustrates the proportion of the lower leg where the post fracture swelling is the highest.

FIG. 28 defines compartments of the upper leg.

FIG. 29 illustrates the proportion of the upper leg where the post fracture swelling is the highest.

FIG. 30 illustrates the areas in plantar foot and the likely areas of callus and ulcers development in diabetic foot.

FIG. 31 illustrates a wrist orthotic with an embedded pressure sensor to monitor flexion attempt.

FIG. 32 illustrates a wrist orthotic with a custom embedded pressure sensor to monitor radial deviation attempt.

FIG. 33 illustrates the production technique of personal biomechanical sensors with comfortable body interface.

FIG. 34 illustrates the placement of mems barometers in molding process of personal biomechanical sensors with comfortable body interface.

FIG. 35 illustrates an ankle orthotic with an embedded tension sensor to monitor plantar flexion attempt.

FIG. 36 illustrates a dynamic upper extremity orthotic with an embedded strain sensor and modular beneficiaries.

FIG. 37 illustrates the section of the dynamic upper extremity orthotic with an embedded strain sensor.

FIG. 38 illustrates an external pressure provider with embedded pressure sensor, accelerometer and gyroscopes.

FIG. 39 defines the metacarpals

FIG. 40 illustrates convenient areas to apply external pressure to cause flexion/extension to the wrist.

FIG. 41 illustrates convenient areas to apply external pressure to cause ulnar and radial deviation to the wrist.

FIG. 42 illustrates convenient areas to apply external pressure to cause pronation to wrist and upper extremity.

FIG. 43 illustrates convenient areas to apply external pressure to cause supination to wrist and upper extremity.

FIG. 44 illustrates convenient areas to apply external pressure to cause flexion/extension to the elbow.

FIG. 45 defines the shoulder region in the upper extremity with neighboring skeletal system elements.

FIG. 46 illustrates convenient areas to apply external pressure to cause flexion/extension to the shoulder.

FIG. 47 illustrates convenient areas to apply external pressure to cause abduction/adduction to the shoulder.

FIG. 48 illustrates convenient areas to apply external pressure to cause plantar flexion and dorsiflexion to the ankle.

FIG. 49 illustrates convenient areas to apply external pressure to cause abduction/adduction to the ankle and lower extremity.

FIG. 50 illustrates convenient areas to apply external pressure to cause flexion/extension to the knee.

FIG. 51 defines the hip joint with neighboring skeletal system elements.

FIG. 52 illustrates convenient areas to apply external pressure to cause flexion/extension to the hip joint.

FIG. 53 illustrates convenient areas to apply external pressure to cause abduction/adduction to the hip joint.

FIG. 54 illustrates convenient areas to apply external pressure to cause medial rotation/lateral rotation to the hip joint.

FIG. 55 illustrates the use of the diagnostic probe on an ankle orthotic in foot abduction.

FIG. 56 is a wrist orthotic with a plurality of sensors/accelerometers to monitor anatomic correlation patterns of different exercises trough a guided learning mode.

FIG. 57 illustrates a modular adjustable external pressure provider unit.

FIG. 58 illustrates the use of modular adjustable external pressure provider units in spinal orthotics.

FIG. 59 provides more detail in the use of modular adjustable external pressure provider units in spinal orthotics.

FIG. 60 illustrates a therapeutic splint system with, modular probe stabilizers, probes, cables, and an energy source generator.

FIG. 61 illustrates a therapeutic splint with solid heat distribution system, modular probe stabilizers, probes, cables, and an energy source generator.

FIG. 62 illustrates a therapeutic splint system with liquid heat distribution system working with liquid cooling or heating equipment.

FIG. 63 illustrates the liquid flow in the therapeutic splint system.

FIG. 64 illustrates an upper extremity splint with a passive dermal pharmaceutical administration patch (storage).

FIG. 65 illustrates an upper extremity splint with an active dermal pharmaceutical administration with ventilation holes, a needle, a pharmaceutical liquid compartment, a microcomputer, batteries, and wiring to provide medication on demand.

### DETAILED DESCRIPTION OF THE INVENTION

The present invention relies on known techniques for manufacturing personal three-dimensional printed orthotics which generally utilize three steps. The first step is reference geometric data gathering; the goal of this step is capturing the anatomic geometry of the patient. Relevant three-dimensional scanning or medical imaging technologies are used in order to capture the personal three-dimensional geometry. This three-dimensional geometry later provides the basis for the interior geometry of the custom three-dimensional printed orthotic. The second step is personal splint design; the goal of this step is determining and modeling the physical structure of the custom three-dimensional printed orthotic. During this step, design features of the orthotic, such as hinges, large window openings (any three-dimensional modification applicable to the orthotic structure within traditional methods applied in plaster and thermoplastics) can be marked on a patient's skin with ink markers for a CAD designer to follow as instructions. In particular, the locations for the sensor target regions can be marked so that they are carried over into the scanned three-dimensional data set. The third step is three-dimensional printing of the personal orthotic. Any known three-dimensional printing technology can be used for the task.

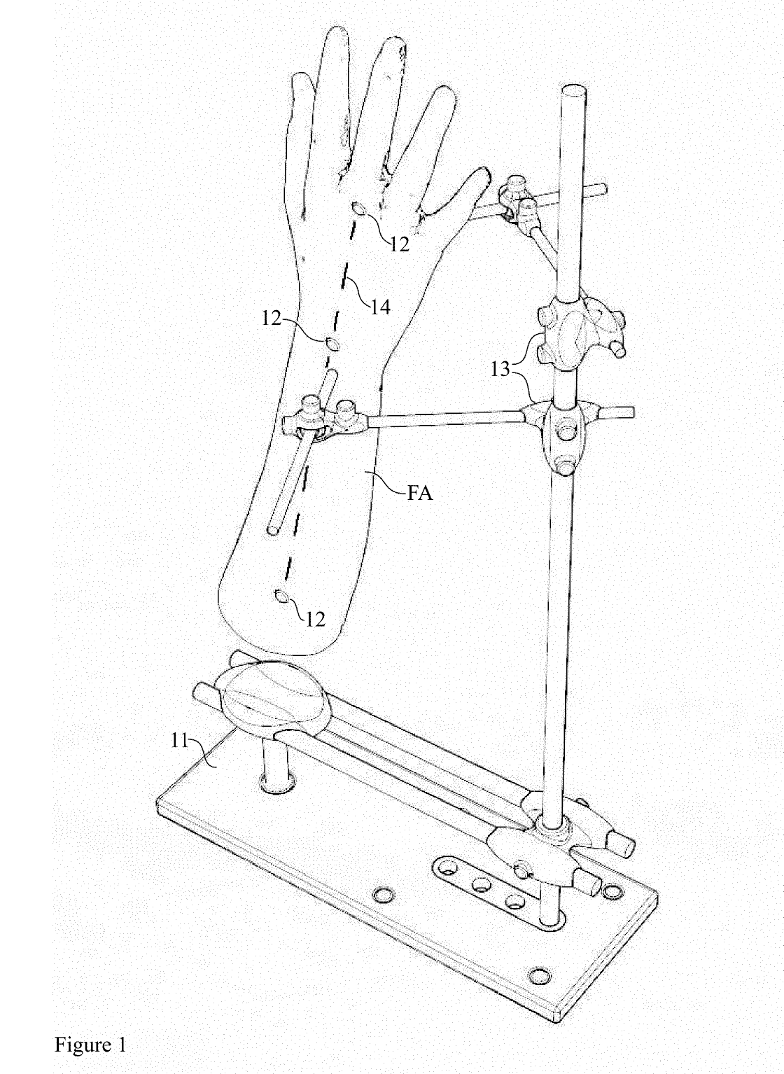

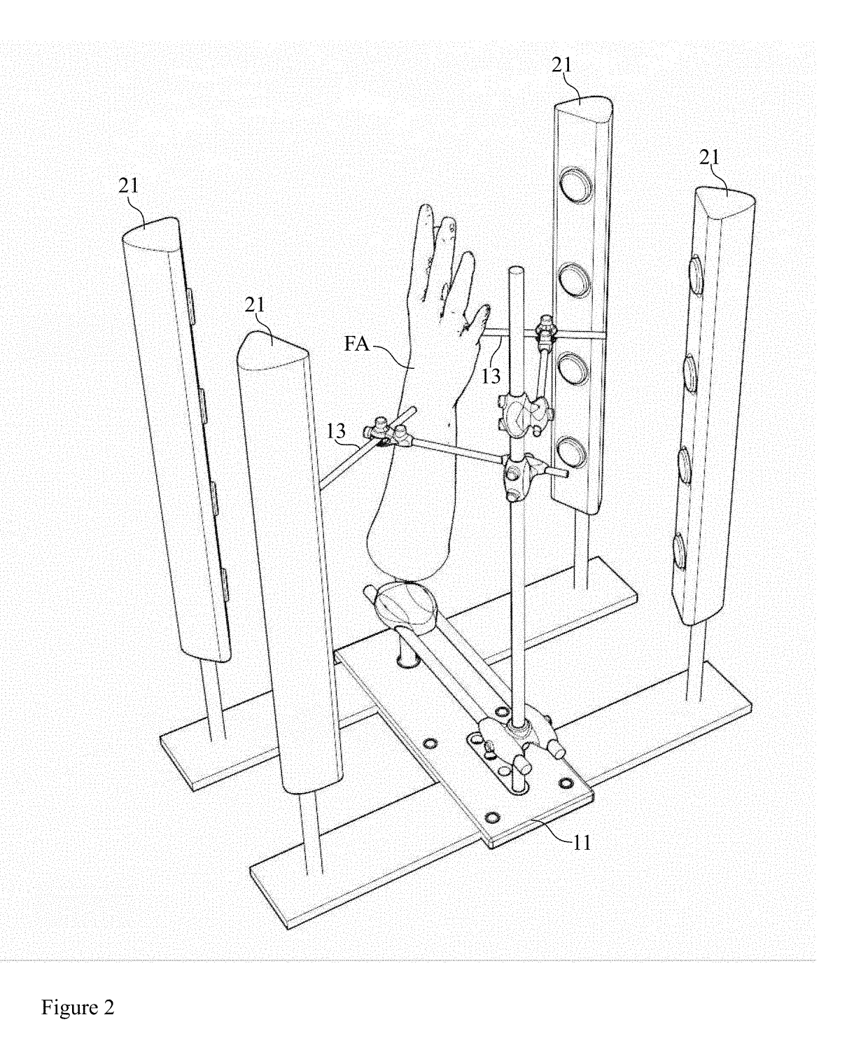

With further reference to geometric data gathering, this stage presents the foundation of the entire process as it is the geometric reference of the three-dimensional printed personal diagnostic device. The goal of this stage is to capture patient's desired (relevant) anatomic region with the use of three-dimensional scanning technologies. Depending on the anatomic location and the three-dimensional scanning technology available, this process can take between a few seconds to a few minutes. The relevant anatomic region of the patient is usually stabilized during scanning, and depending on patient's medical condition or physical limitations, patients may need different levels of orthotic intervention, support, and assistance during this step. Although stabilization and the three-dimensional scanning process can be exactly the same among different anatomic regions, the following example is specifically designed for upper extremity cases such as a forearm FA (FIG. 1). The scanning apparatus includes an elbow rest (11) and adjustable external orthotic intervention platforms (12). With this equipment, the forearm is stabilized by applying pressure from multiple supports or fiducials (13) aligned along an axis 14 of the forearm. Surrounding three-dimensional imaging equipment such as scanning towers 21 (FIG. 2), can be used to efficiently three-dimensionally scan the desired areas. Such scanning and assistance systems incorporate three-dimensional imagery equipment (cameras, lasers), physical intervention mechanisms, data processing units, wireless communication units, data storage, on board or remote interface through software to assist, monitor and guide the physical positioning process.

Commercially available computer aided design (CAD) software, such as Autodesk Fusion 360, Rhinoceros 5, and Solid Works, can be used to import the patient's three-dimensional body surface geometry and to design a body scaffold suitable for incorporation into the conformable body interfaces of the present invention. Usually, a medical professional skilled in designing orthotics or other body splints and a CAD designer will work together in designing an orthosis for an individual patient. The medical professional positions the patient's anatomy and marks interventional and locational information on the patient's skin with ink markers. The CAD designer follows these instructions during the CAD design stage. The medical professional can mark important orthotic design considerations with a combination of different colored markers and line types (straight lines, dashed lines, dotted lines, specific symbols, shapes etc.). Such orthotic design considerations (inputs) provide the limitations of the orthotic, hinges, joining mechanisms, window openings, areas for damping, areas to avoid, areas for sensor placement and areas for therapeutic beneficiary placement. Alternatively, the CAD software can also be modified to trace the marked input as commands with determined anatomic locations and generate the design. Further alternatively, the body scaffold can be designed using software with an augmented or virtual reality interface.

Physical examinations are used for diagnosis and classification of numerous neuromuscular and musculoskeletal systems. An orthopedic examination process can include the following stages: (1) Inspection (surface anatomy, alignment, gait and range of motion) and (2) Palpation/Manipulation (muscle testing, pain sensation testing, reflex testing and stability testing). Diagnostic systems useful in the present invention will usually provide a range of orthopedic examination techniques and digitize these processes in the areas of flexibility/stiffness, muscle strength, range of motion and other relevant examination processes involving palpation/manipulation. These diagnostic systems rely on analyzing biomechanics, orthopedic biomechanics, anatomy and sensory technologies in order to determine the correct types and anatomic locations for sensing motion and other physical and biological data and/or therapeutically intervening. The sensory equipment used include anatomic (pressure, tension, position, etc.) and physiologic sensors (pulse, temperature, oximeter, and the like. The sensed data can be collected and used for both local and remote data analysis. In particular, the data for individual patients and for populations of patients may be collected at central location(s) (e.g. in the “cloud”) and used for individual and population analytics.

The language used to refer to anatomic structures and biomechanical phenomenon as used herein will now be defined. Terms related to anatomic locations are often used to indicate the position of one structure relative to another. Proximal means anatomically nearer to a point of reference such as an origin, a point of attachment, or the midline of the body opposite of distal. Distal means anatomically located far from a point of reference, such as an origin or a point of attachment opposite of proximal. Anterior means anatomically situated at or directed toward the front, in human anatomy, denoting the front surface of the body, that is, situated nearer the front part of the body, opposite of posterior. Posterior means directed toward or situated at the back, denoting the back surface of the body, opposite of anterior. Medial means anatomically situated toward the midline of the body or a structure, i.e. the opposite of lateral. Lateral means a position farther from the median plane or midline of the body or a structure, pertaining to a side, i.e. the opposite of medial. Joint motion is assessed within three planes of movement: the sagittal plane, the frontal plane, and the transverse plane. The sagittal plane passes through the body front to back, thus dividing a body region into left and right. Movements in this plane are the up and down movements referred to as flexion and extension. The frontal plane divides the body into front and back or anterior and posterior. Movements in this plane are sideways movements, referred to as abduction and adduction. The transverse plane divides the body into top and bottom or superior and inferior. Movements in this plane are rotational in nature, such as internal and external rotation, pronation, and supination. Flexion and extension describe movements that affect the angle between two parts of the body. Flexion describes a bending movement that decreases the angle between a segment and its proximal segment. Extension is the opposite of flexion, describing a straightening movement that increases the angle between body parts. Abduction refers to a motion that pulls a structure or part away from the midline of the body. In the case of fingers and toes, it refers to spreading the digits apart, away from the centerline of the hand or foot. Abduction of the wrist is also called radial deviation. Adduction refers to a motion that pulls a structure or part toward the midline of the body, or towards the midline of a limb. In the case of fingers and toes, it refers to bringing the digits together, towards the centerline of the hand or foot. Several joints are capable of movements that resist being forced into this system of classification. This has given rise to other descriptive terms particular to specific parts of the anatomy, such as opposition, inversion/eversion, and pronation/supination.

All orthotic designs are based on three relatively simple principles: pressure, equilibrium and the lever arm principle. The “equilibrium principle” is that the sum of the forces and the bending moments created must be equal to zero. The “lever arm principle” is that the further a point of force is from the joint, the greater the moment arm and the smaller the magnitude of force required to produce a given torque at that joint. The present invention relies heavily on these principles to digitize the biomechanics of orthopedic evaluation and rehabilitation processes. Static orthosis have no moveable joints incorporated in to the design. However, a static orthosis may allow active joint motion in one direction, but block motion in another direction (static with a block). A static orthosis may also be changed or adjusted to alter motion allowed or alter the pressure across a joint for stretching purposes (progressive static). Dynamic orthoses have movable joints that can limit motion (block), increase motion through traction, or substitute for weak muscles using supplemental force (assist).

Static custom three-dimensional printed orthotic structures or “splints,” referred to as static body scaffolds,” according to the present invention will provide an external force to counter act imbalances of internal forces resulting from joint motion or instability. The sensors and diagnostic systems of the present invention are incorporated into the scaffolds to measure and “digitize” the biomechanical forces caused by incipient anatomic motion (flexion, extension, deviation, rotation, pronation, supination and other accumulation of pressure, tension and torque) within the areas under orthotic intervention. The detection of such internal forces can be indicative or diagnostic of a variety of conditions such as spasticity, brain damage, nerve damage, cerebral palsy, strokes, arthritis, carpal tunnel syndrome, scoliosis, lordosis and kyphosis. Specific conditions may be of congenital or non-congenital origins and may be triggered by neuromuscular and/or musculoskeletal reactions, such as stiffness and contractures.

Incipient anatomic motion is particularly useful for monitoring spasticity in muscles. It is known to evaluate spasticity by applying force to joints and judge the counter force caused by spasms. By applying a constant pressure to the joint using a body scaffold in accordance with the present invention, the anatomy is trained to neutralize the same imbalances. Alternatively occurrence of such internal forces may cause by voluntary muscle tension/compression. Digitization of voluntary attempts of anatomic motion is a convenient method for monitoring the progress of the changes in muscle strength, for example from degenerative illnesses such as multiple sclerosis (MS), and the like. A dynamic relationship between the orthotic or other body scaffold and the patient anatomy provides sensory locations to create a diagnostic tool for patients with related disorders and digitize current subjective methods of evaluation. A pressure profile will occur within the scaffold on an anatomic plane of the intended motion. The present invention relies on the equilibrium principle to determine the anatomic location of each pressure point. The scaffolds will usually provide a three point pressure equilibrium where the highest pressure point occurs is located on a side of the joint opposite to the direction of the intended motion. The other two pressure points occur in the orthotic distal and proximal to the patient's anatomy, located on the direction of the intended motion. Although orthotics with four or more pressure points exist, the lever arm and the pressure principles are universal for all orthotics. The diagnostic systems of the present invention utilize sensory locations within the scaffolds in tandem with equilibrium principle within static orthotic structures in relation to internal imbalances.

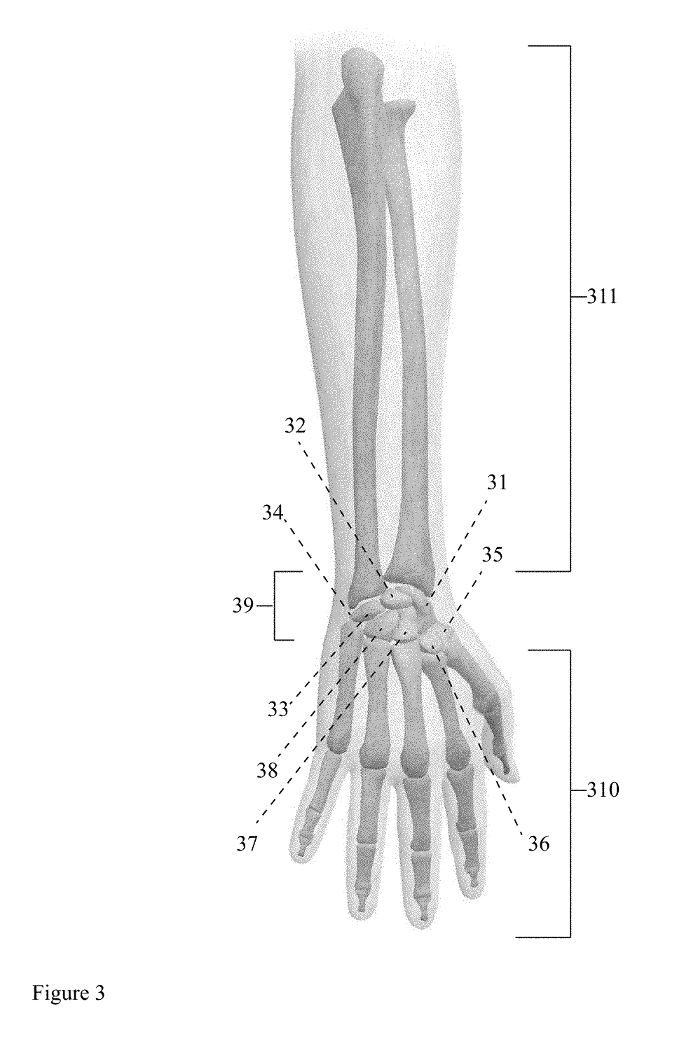

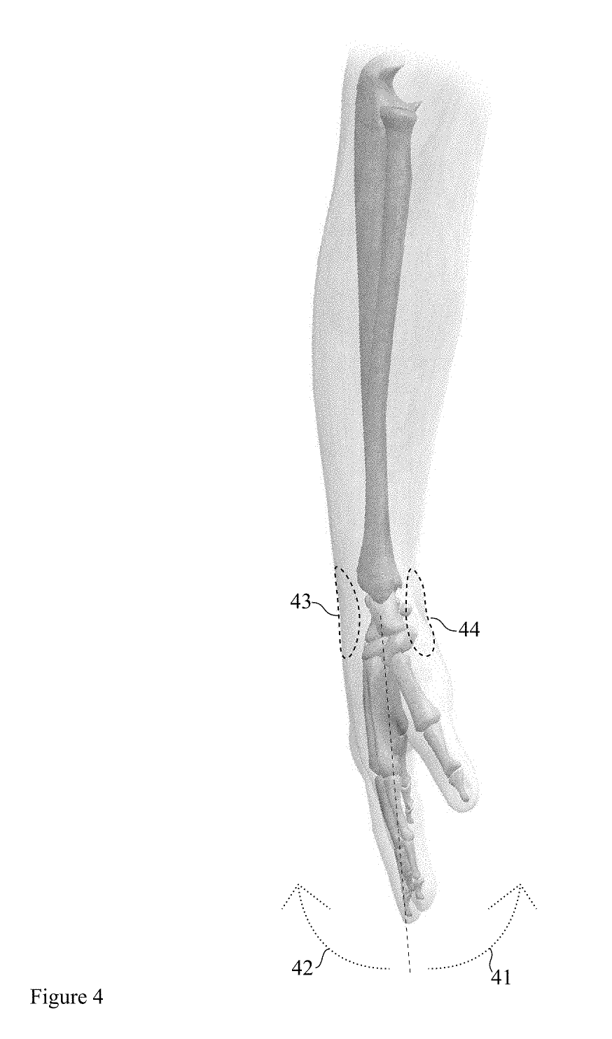

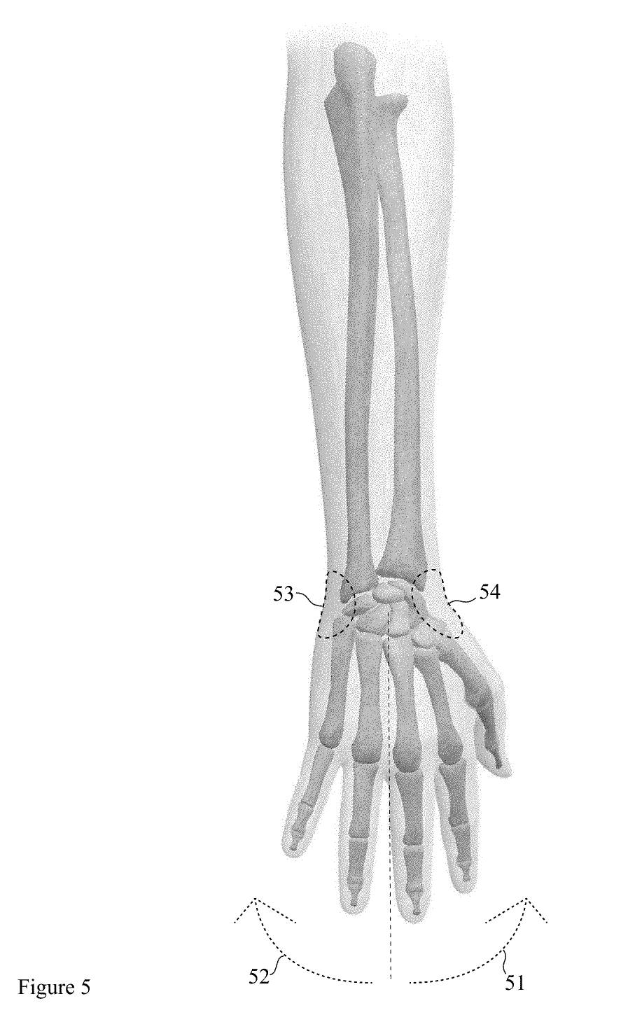

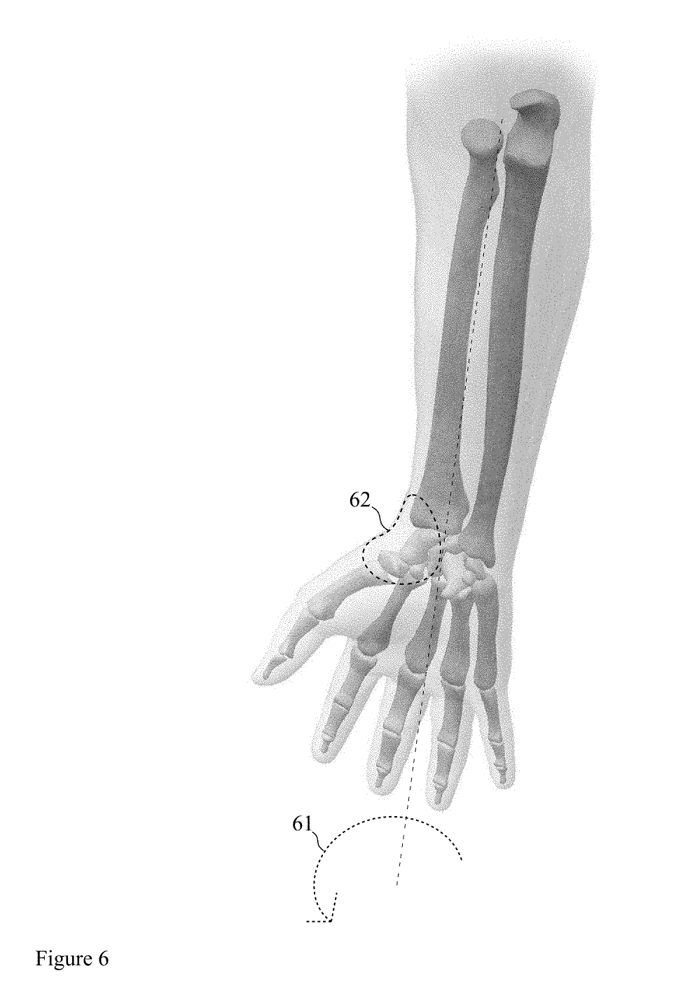

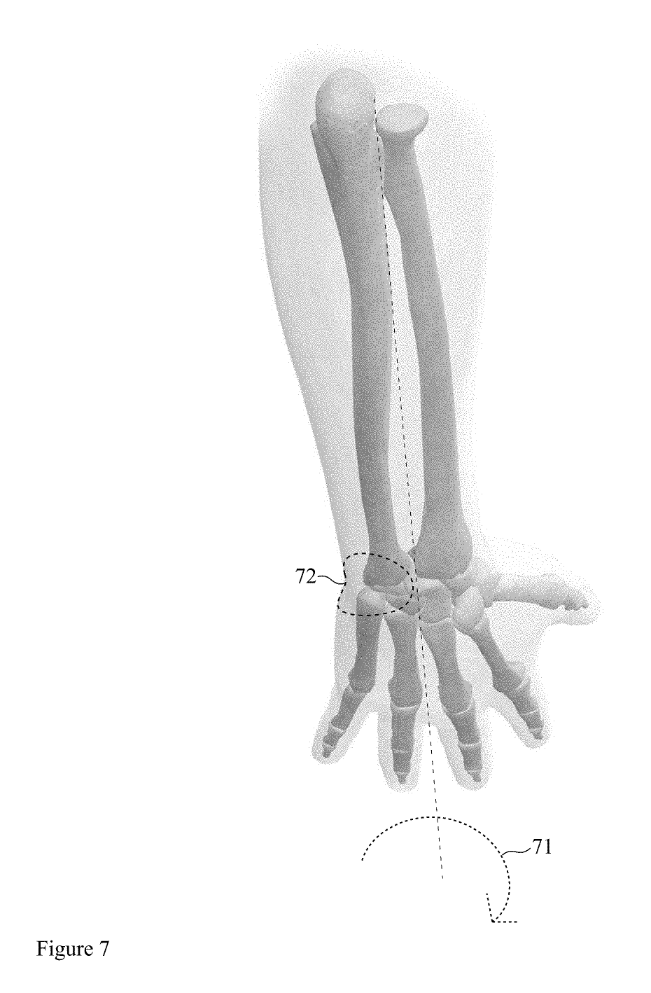

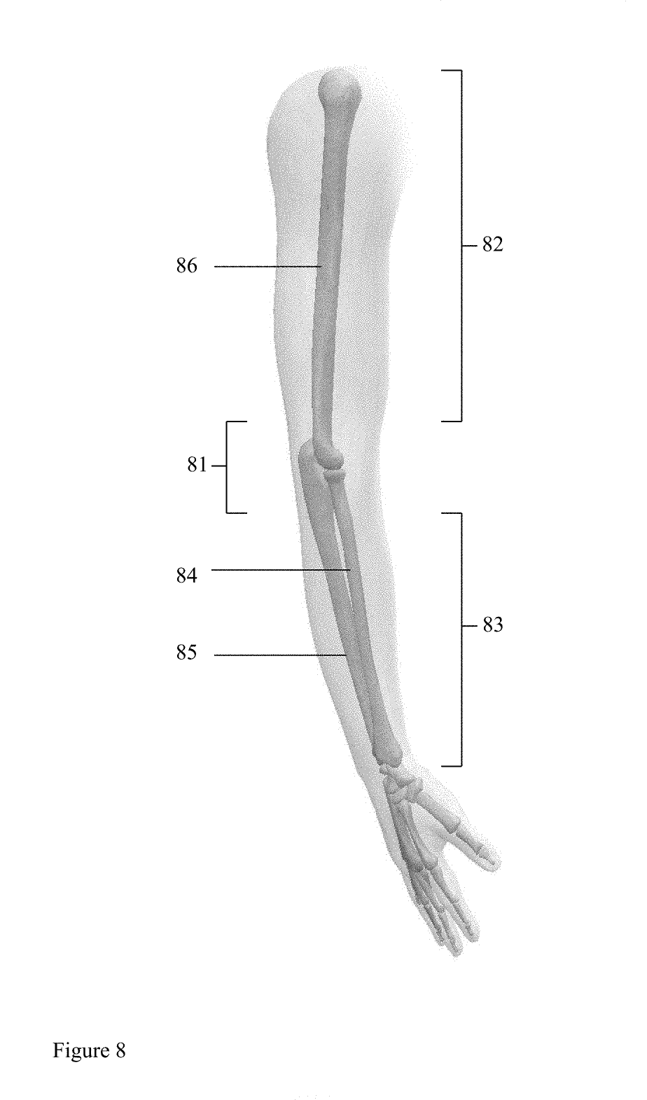

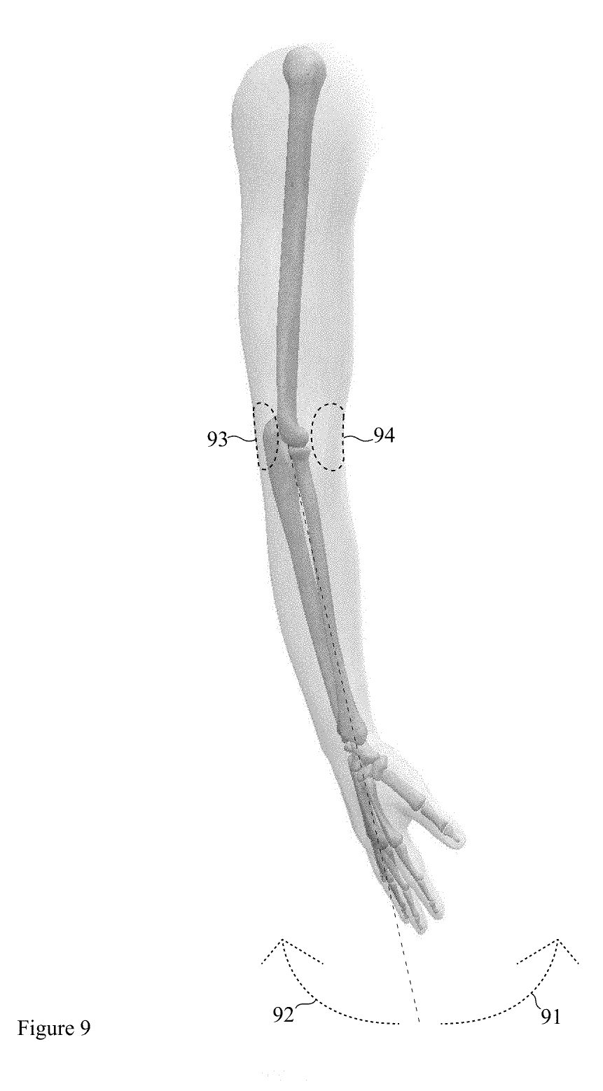

“Anchor” points useful in the present invention for locating pressure and tension sensors for upper extremity body scaffolds and splints, e.g. for the wrist and elbow, are described in FIGS. 3-9. As described elsewhere herein, the pressure and tension sensing will often be correlated with the detection of incipient motion but may find other uses as well. Referring to FIG. 3, a wrist joint (carpals) (39) is between hand (310) and forearm (311). The wrist joint contains eight carpal bones: scaphoid (31), lunate (32), triquetral (33), pisiform (34), trapezium (35), trapezoid (36), capitate (37) and hamate (38). Wrists can move in three each of the three different anatomic planes; sagittal, frontal and transverse planes. Incipient wrist motion in the sagittal plane is illustrated in FIG. 4 including of flexion (arrow 41) and extension (arrow 42). In incipient flexion (41), the highest pressure profile will occur in posterior carpal region (43.) In incipient extension (42), the highest pressure profile will occur in anterior carpal region (44). Incipient wrist motion in the frontal plane is illustrated in FIG. 5 and includes radial deviation (51) and ulnar deviation (52). In incipient radial deviation a (51), the highest pressure profile will occur in lateral carpal region (53). In incipient ulnar deviation (52), the highest pressure profile will occur in medial carpal region (54). Incipient wrist motion in the transverse plane is illustrated in FIGS. 6 and 7 include pronation (61) and supination (62). In incipient pronation (61), the highest pressure will occur in anterior-medial carpal region (62). In incipient supination (71), the highest pressure will occur in the posterior-lateral carpal region (72). Referring to FIG. 8, an elbow (81) is the joint between upper arm (82) and forearm (83), formed at a junction between (1) the proximal radius (84) and proximal ulna (85) with (2) the distal humerus (86). The elbow moves in only the sagittal plane. Incipient motion for elbow in the sagittal plane is illustrated in FIG. 9 and includes of flexion (91) and extension (92). In incipient flexion (91), the highest pressure will occur in a posterior elbow region (93). In incipient extension, (92), the highest pressure profile will occur in anterior elbow region (94).

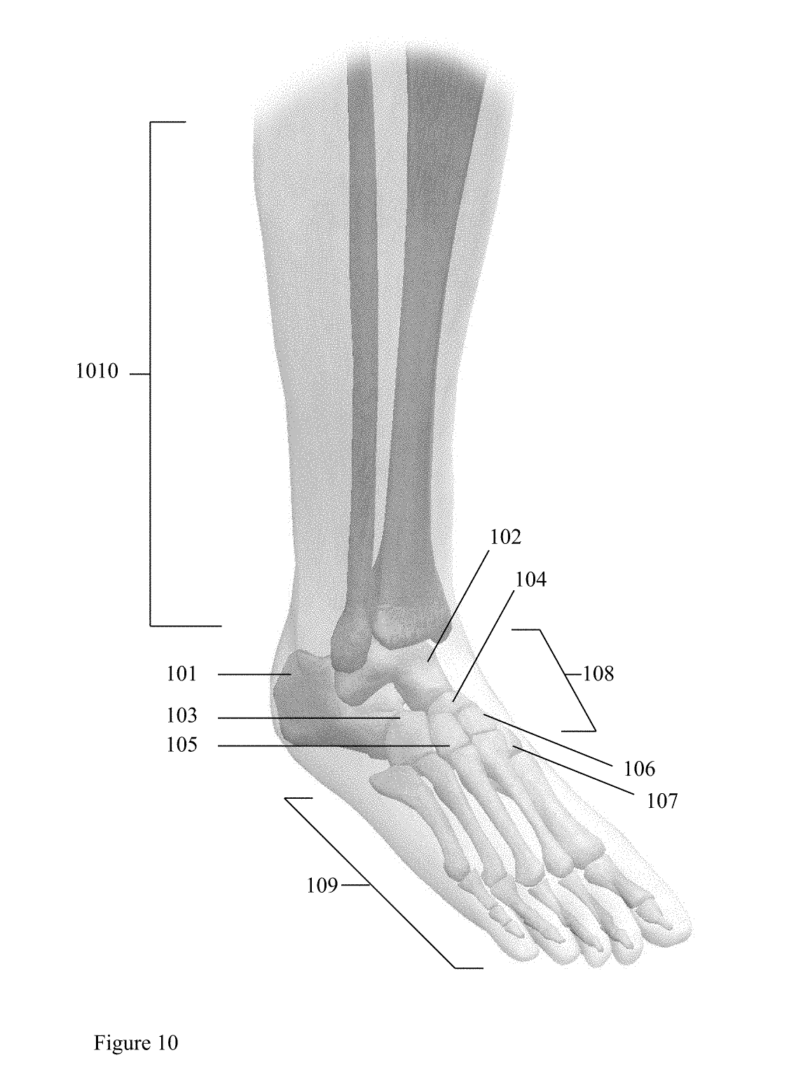

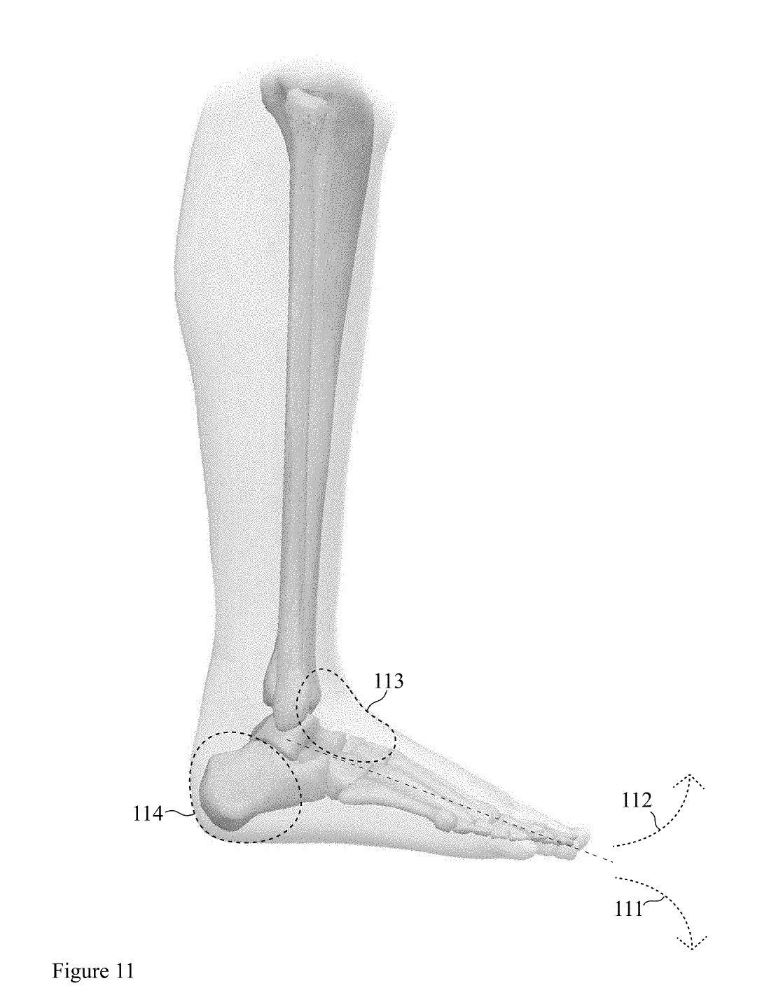

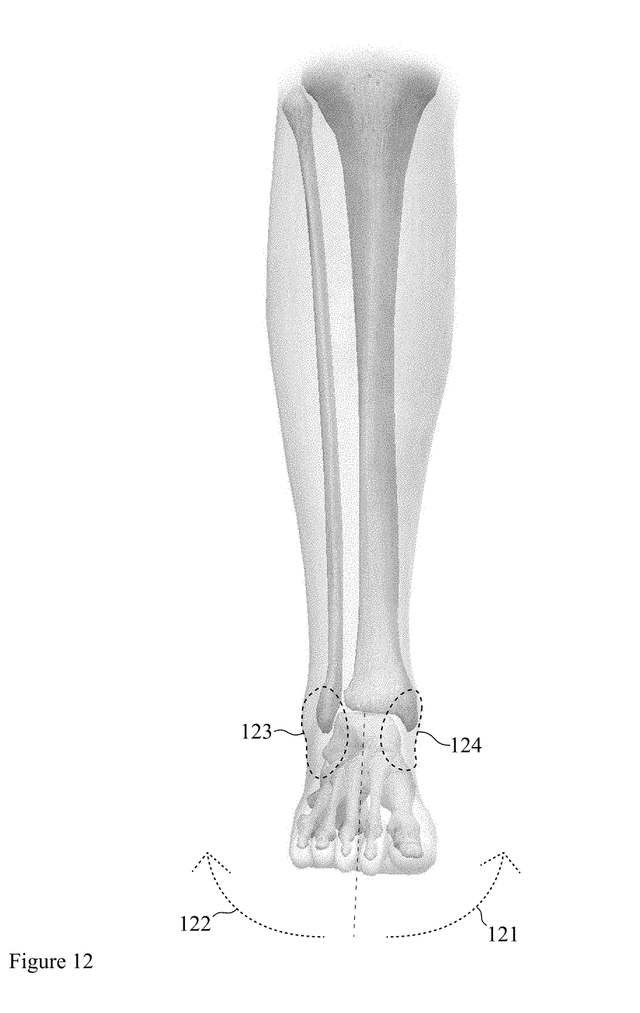

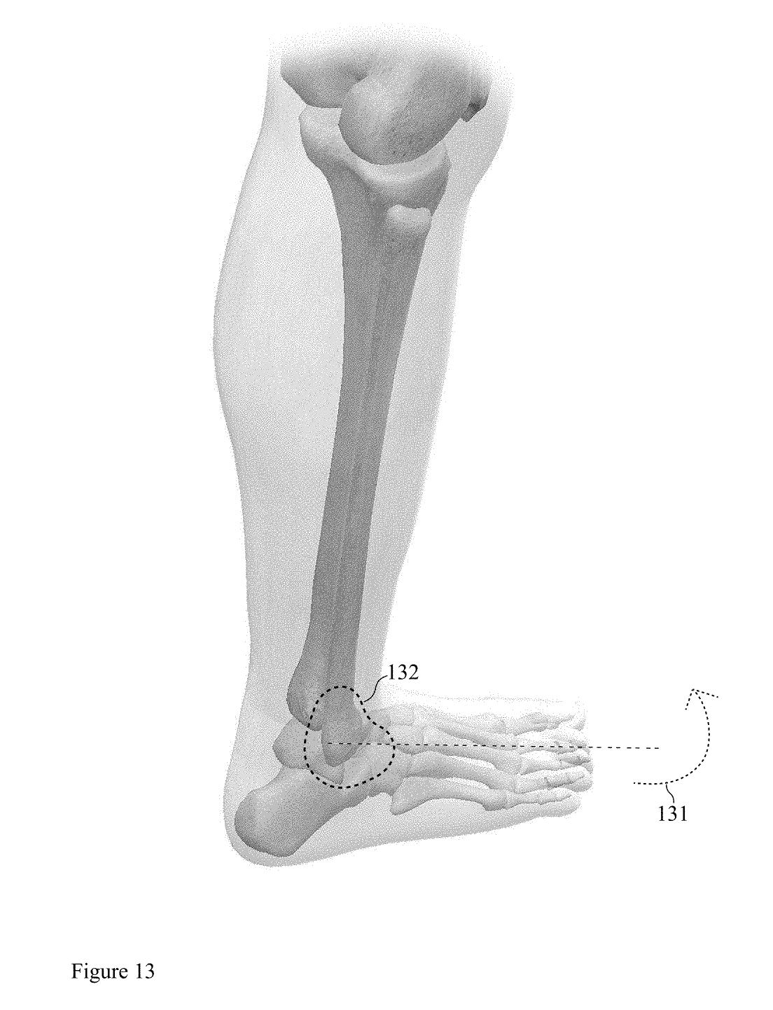

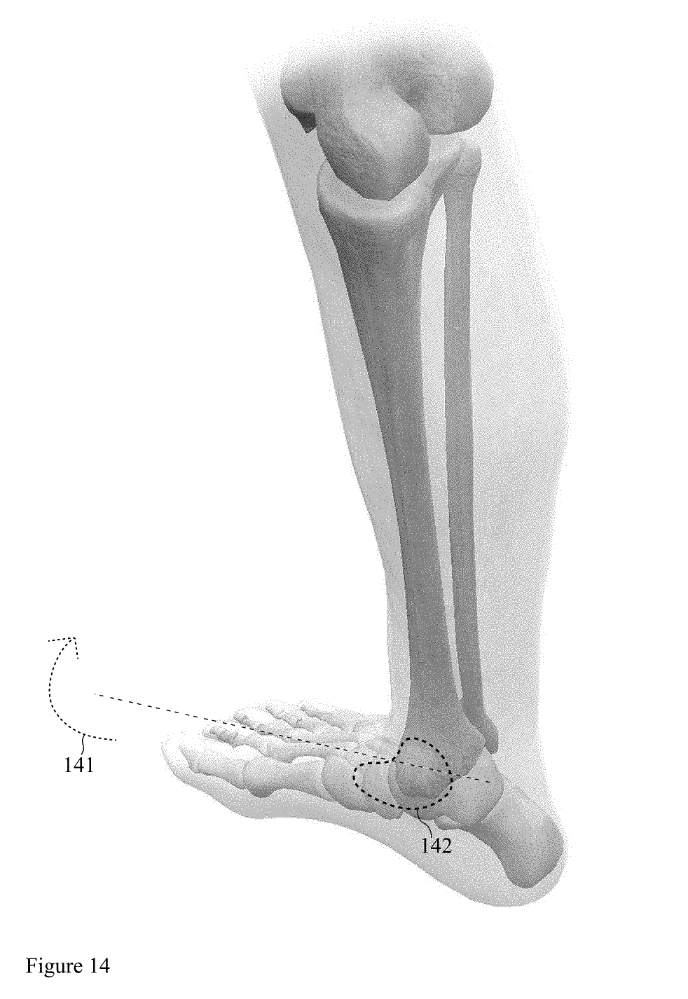

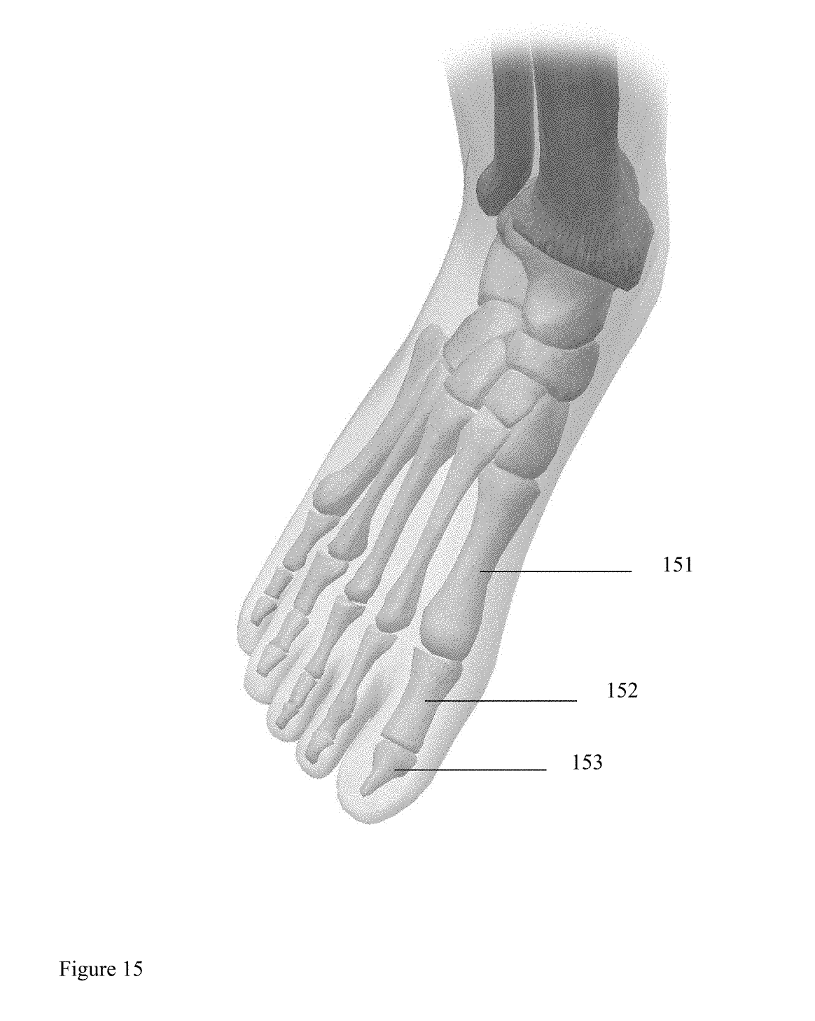

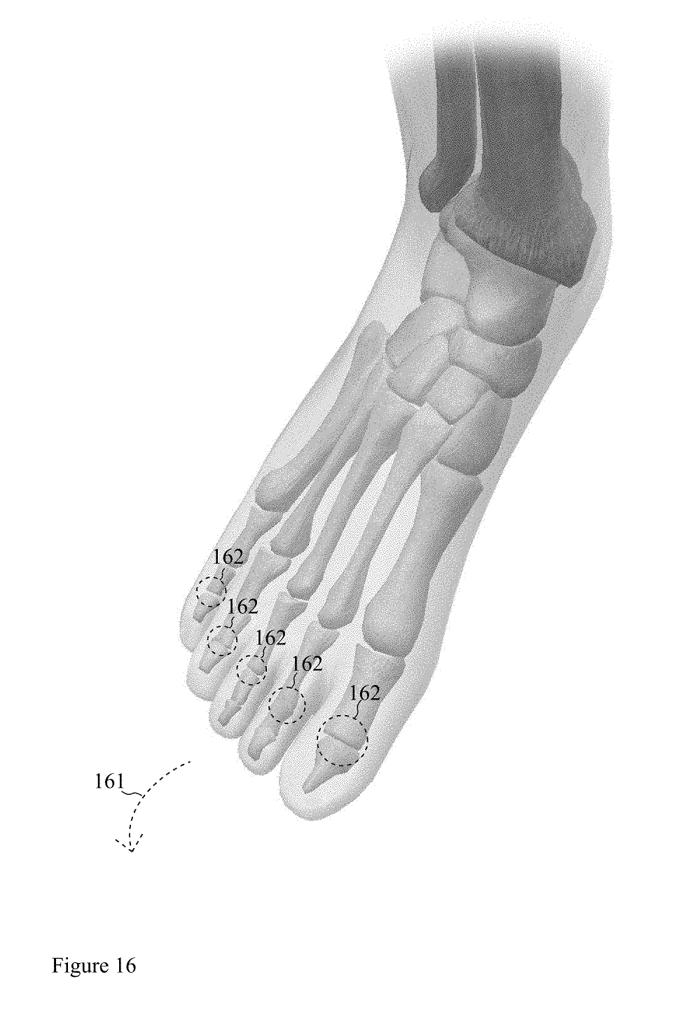

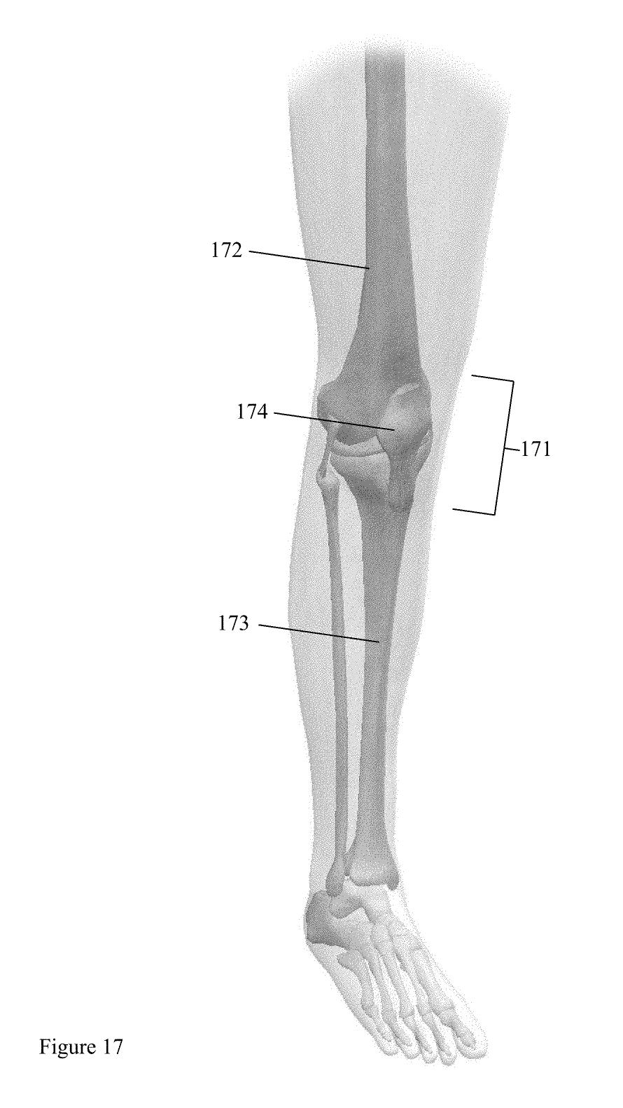

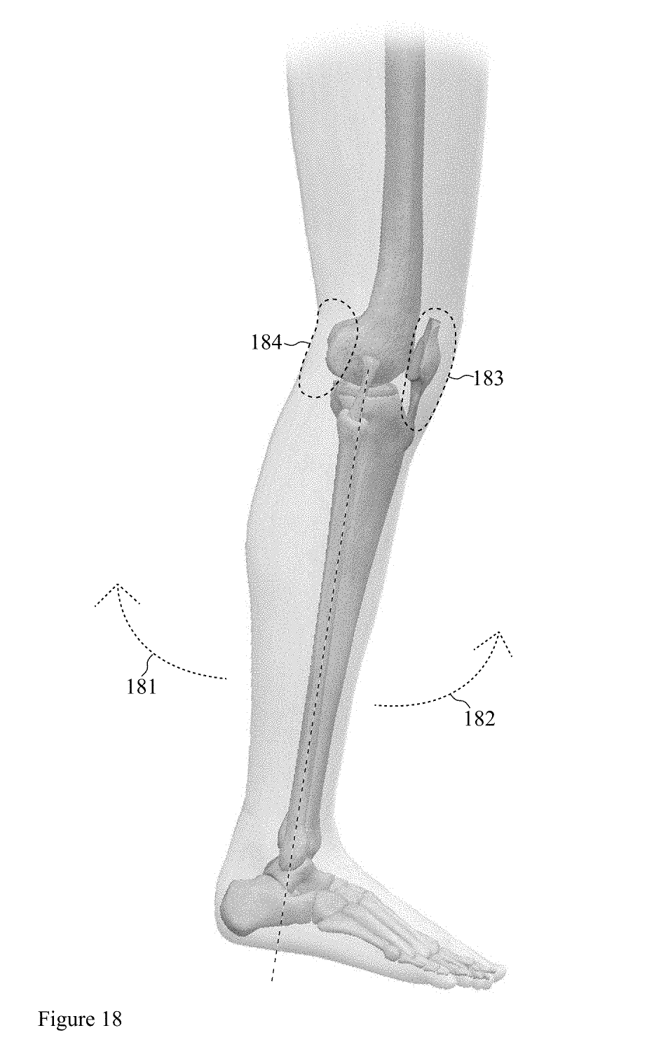

“Anchor” points useful in the present invention for locating motion sensors (e.g. pressure and tension sensors) in lower extremity body scaffolds and splints are described in FIGS. 10-18. Referring to FIG. 10, an ankle joint is present in the tarsals region (108) and formed by the articulation of the lower leg bones (1010) with the talus. The ankle connects the foot (109) with the leg bones (1010). The tarsus is a cluster of seven articulating bones: the calcaneus (101), talus (102), cuboid (103), navicular (104), lateral cuneiforms (105), intermediate cuneiform (106), navicular (107). As shown in FIG. 11, incipient motion in the sagittal plane includes plantar flexion (111) and dorsiflexion (112). During plantar flexion (111) the highest pressure occurs in posterior ankle region (114) (medial malleoli, extensor retinaculum, and lateral malleolus). In dorsiflexion (112), the highest pressure occurs in anterior ankle region (113) (medial malleoli, calcaneus, and lateral malleolus). As shown in FIG. 12, incipient motion in the frontal plane includes inversion (121) and eversion (122). In inversion (121) the highest pressure will occur in lateral tarsal and lateral-posterior metatarsal region (123) (lateral malleolus, cuboid and plantar 5th metatarsal). In eversion (122), the highest pressure will occur in the medial tarsal region (124) (medial malleolus, medial talus, medial navicular and medial cuneiform). As shown in FIG. 13, a first incipient ankle motion in the transverse plane is supination (131) where the highest pressure will occur in lateral-posterior tarsal region (132) (lateral malleolus, lateral calcaneus, lateral-posterior cuboid and cuneiform). As shown in FIG. 14, a second incipient ankle motion in the transverse plane is pronation (141) where the highest pressure will occur in medial-anterior tarsal region (142) (medial malleolus, medial talus, medial navicular and medial cuneiform). Incipient motion in a patient's toes is illustrated in FIGS. 15 and 16. Toes are digits of the foot, the toe bones are phalanges proximal (151), phalanges mediae (152), and phalanges distales (153). Incipient flexion (161) in the sagittal plane results in highest pressure will occur in the interphalangeal joint areas (162). Referring to FIGS. 17 and 18, the knee (171) is the joint of the leg that allows for movement between the distal femur (172) and proximal tibia (173) and is protected by the patella (174). Incipient motion in the sagittal plane includes flexion (181) and extension (182). The highest pressure in flexion will occur in anterior knee region (183) (patella). In extension (182), the highest pressure will occur in posterior knee region (184) (posterior-proximal tibia and posterior-distal femur).

Although toes can move in multiple anatomic planes the diagnostic system of the present invention is particularly useful for monitoring incipient motion in patients having “hammer toe” or “claw toe” which are deformities which appear in sagittal plane Hammer toe (or claw toe) result from continuous flexion of the proximal interphalangeal joints in the sagittal plane. Incipient motion of the toe in the sagittal plane is illustrated in FIG. 16.

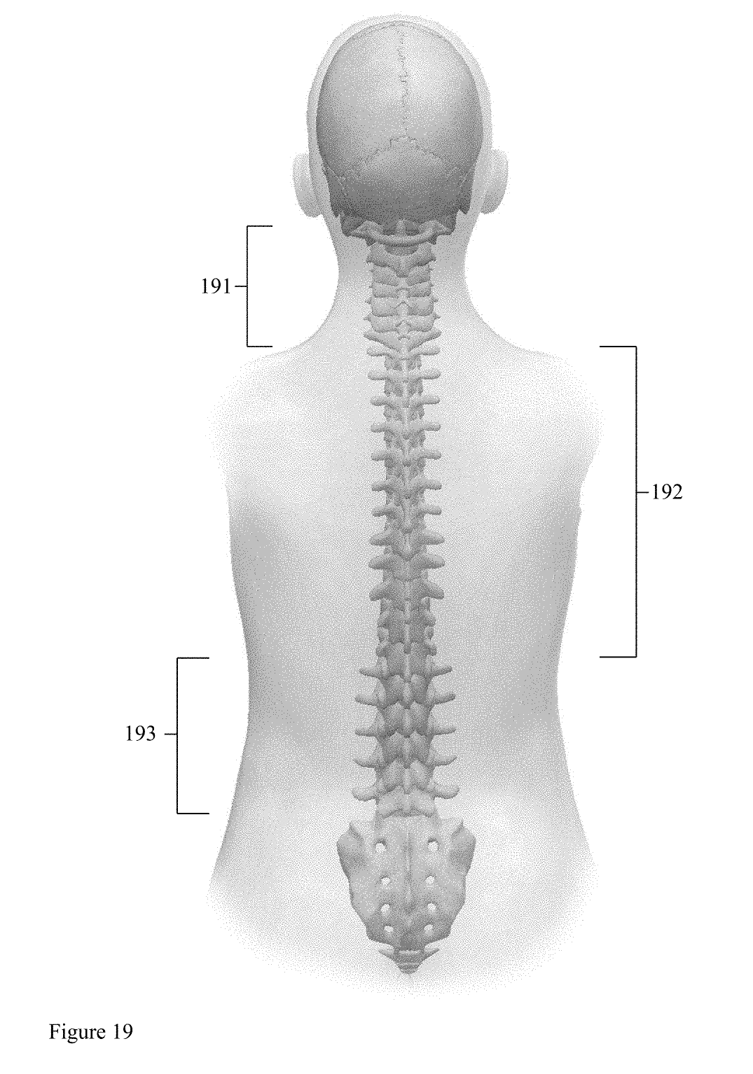

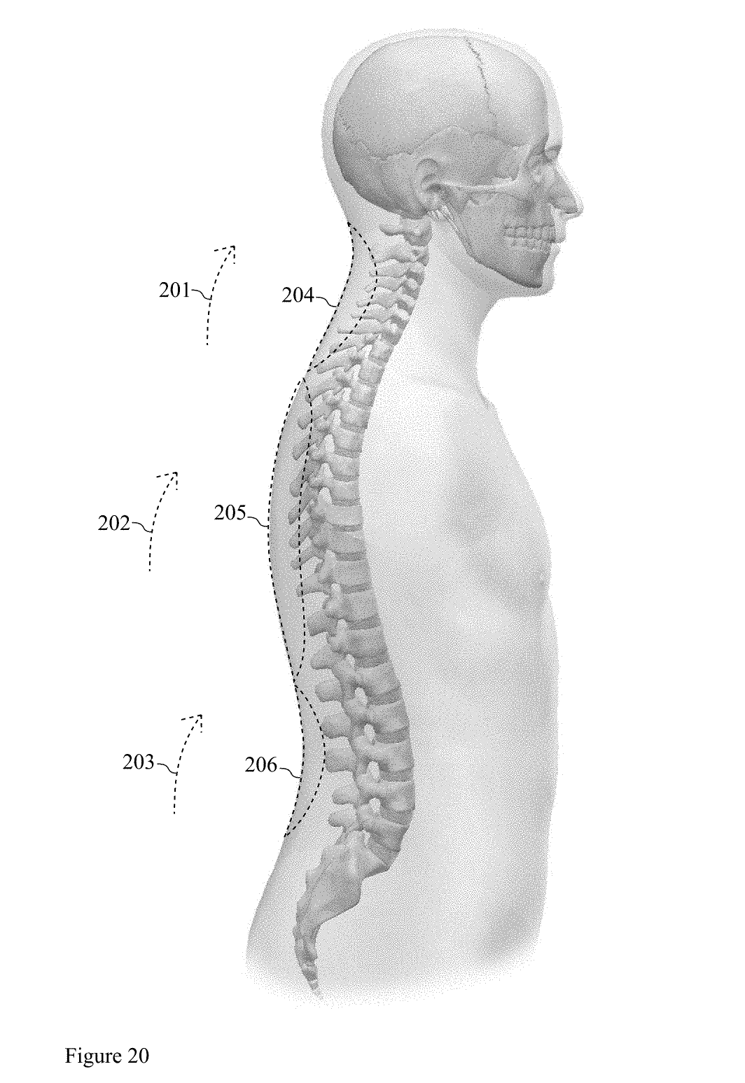

Conventional spinal orthotics may be classified according to the anatomical areas to which they are applied. Referring to FIG. 19, the spine includes three major sections: the cervical vertebrae 191, the thoracic vertebrae 192, and the lumbar vertebrae 193. Each section includes individual bones, called vertebrae. There are seven cervical vertebrae (191), twelve thoracic vertebrae (192), and five lumbar vertebrae (193). Although spine can move in all three planes, the diagnostic systems of the present invention will usually be intended to monitor incipient motion in the sagittal and frontal planes. Incipient motion in the sagittal plane is illustrated in FIG. 20 and includes flexion in each of the three spinal sections. In incipient flexion 201 in the cervical spine (191), the highest pressure profile will occur in posterior cervical spine region (204). In incipient flexion (202) in the thoracic spine (192), the highest pressure will occur in posterior cervical, thoracic region (205). In incipient flexion in the lumbar spine (193); highest pressure will occur in posterior lumbar spine region (206).

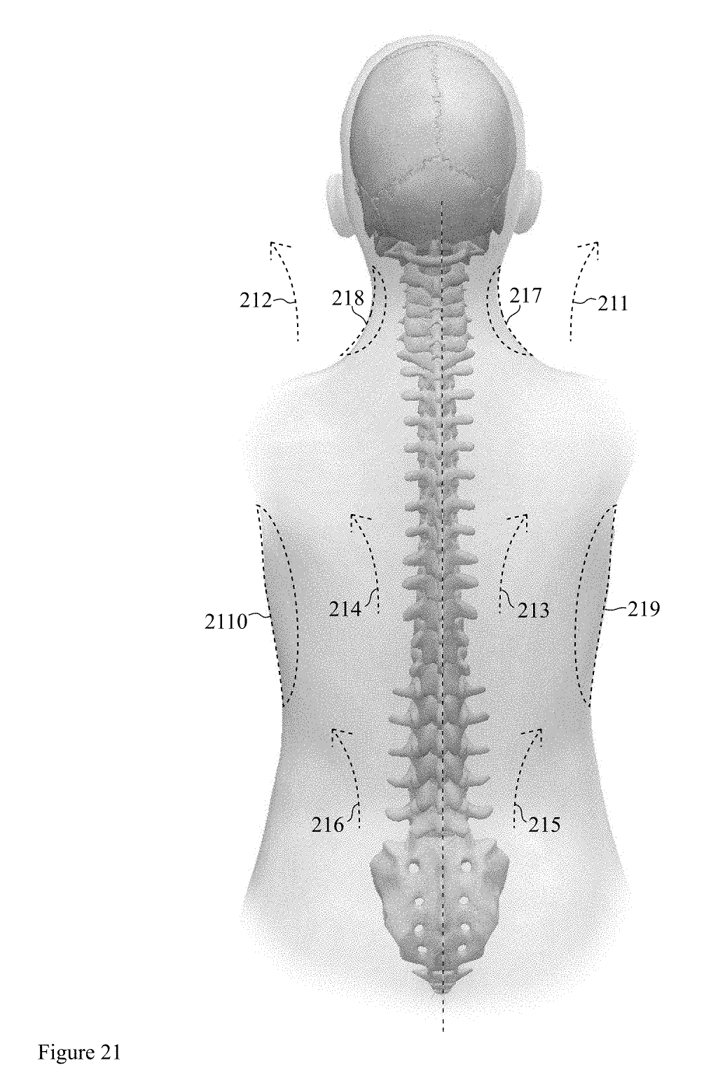

Incipient motion of the spine in the frontal plane is illustrated in FIG. 21 and includes flexion to left and right in the three different sections of the spine. In right incipient lateral flexion (211) in the cervical spine, the highest pressure will occur in right lateral neck area (217). In left incipient lateral flexion (212) in the cervical spine, the highest pressure will occur in left lateral neck area (218). In right incipient lateral flexion in the thoracic spine (213), the highest pressure will occur in right lateral thoracic cage region (219). In left lateral incipient flexion in the thoracic spine (214), the highest pressure will occur in left lateral thoracic cage region (2110). In a right incipient lateral flexion attempt in the lumbar spine (215), the highest pressure profile will occur in right lateral thoracic cage region (219). In a right incipient lateral flexion (216) in the lumbar spine, the highest pressure profile will occur in right lateral thoracic cage region (2110). Although the anatomic regions with the highest pressures will be the most convenient locations to monitor pressure, other pressure points which appear in distal and proximal orthotics may also be suitable locations for pressure monitoring. Also, while the pressure can be measured directly, other forces, strains, displacements, and the like may also be measured at the target locations to assess incipient motion.

Properly placed sensors can also be used to assess pressure accumulation due to swelling or other conditions. The internal body pressure which causes or results from swelling differs from that resulting from incipient joint motion and can be measure with differently located pressure, strain, force, and other sensors located on a body scaffold. Swelling, including turgescence and tumefaction, is a transient abnormal enlargement of a body part or area not caused by proliferation of cells. It is usually caused by an accumulation of fluid in tissues. It can occur throughout the body (generalized), or can be localized in a specific body part or organ.). A body part may swell in response to injury, infection, or disease. Swelling, especially of the ankle, can occur if the body is not circulating fluid well.

In fractures swelling is an autoimmune response, and casts are traditionally fabricated with additional space or volume to accommodate edema. If a cast is too tight, or if the excessive swelling occurs, the patient may suffer compartment syndrome which can in the worst cases result in amputation. The diagnostic systems of the present invention monitor inflammation and swelling by positioning pressure sensors in three-dimensional fabricated body scaffolds. Swelling (edema accumulation) is most intense in the areas where muscle density is highest within muscle compartments. Swelling occurs unevenly within splinted areas. The diagnostic systems of the present invention provide specific sensory locations within static orthotic devices to monitor post trauma swelling (circumferential expansion).

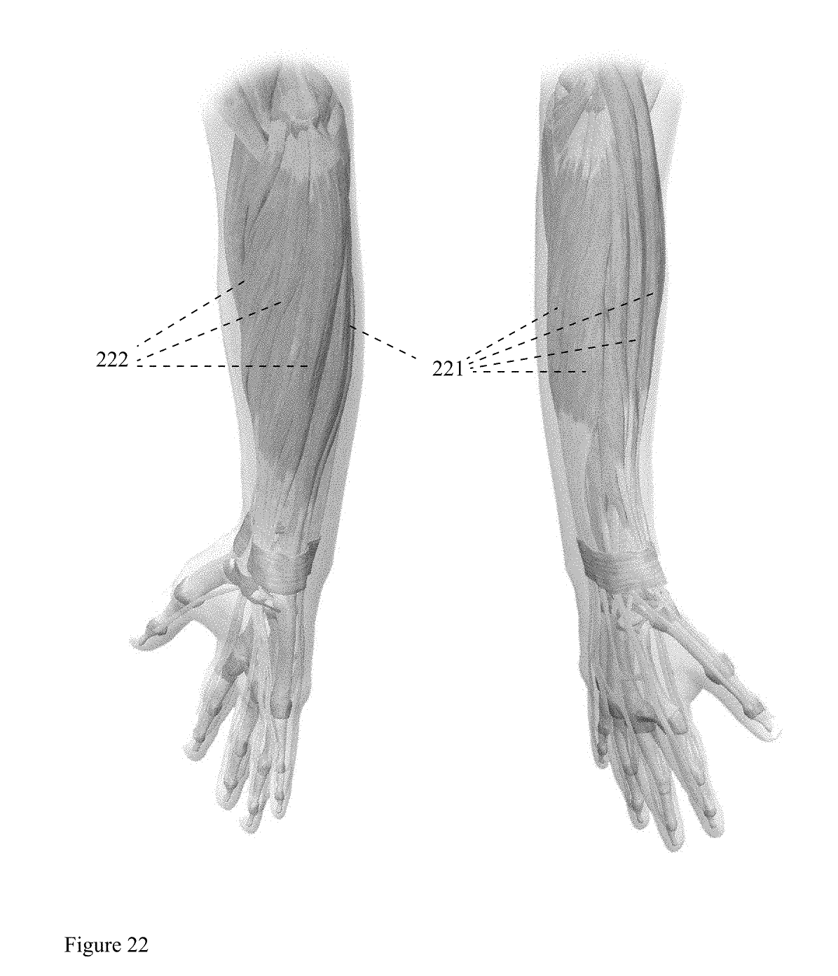

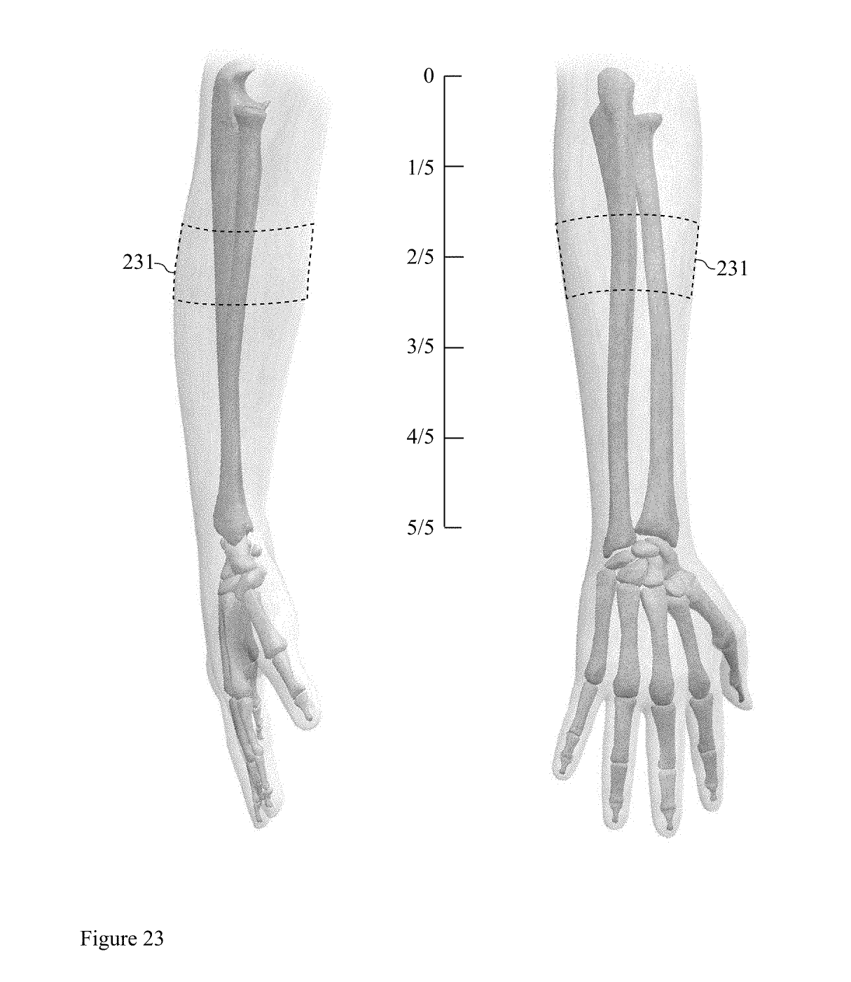

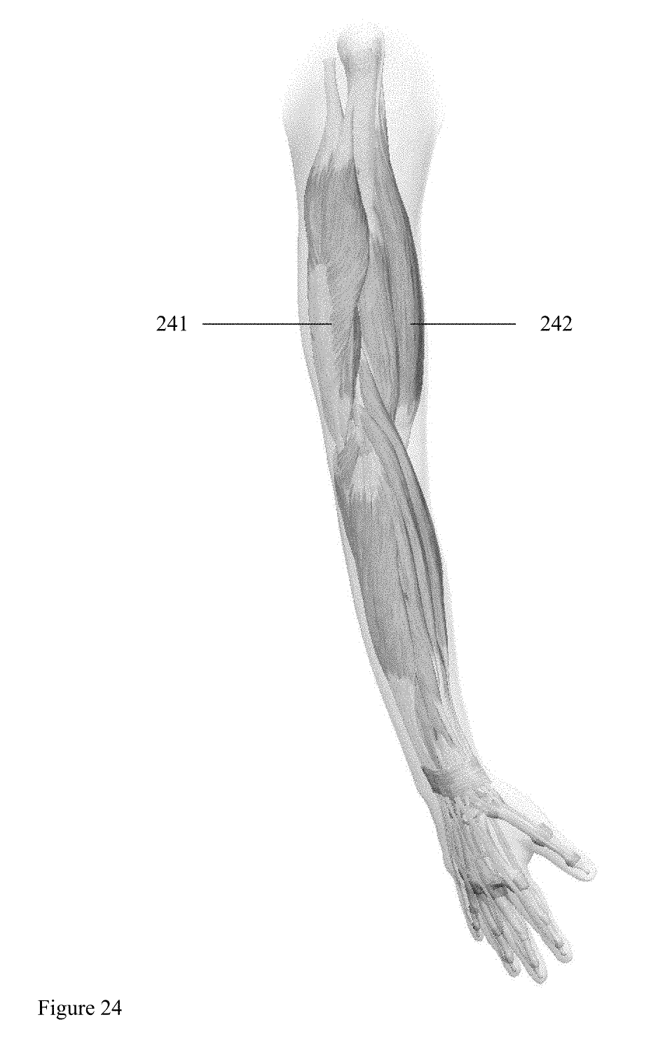

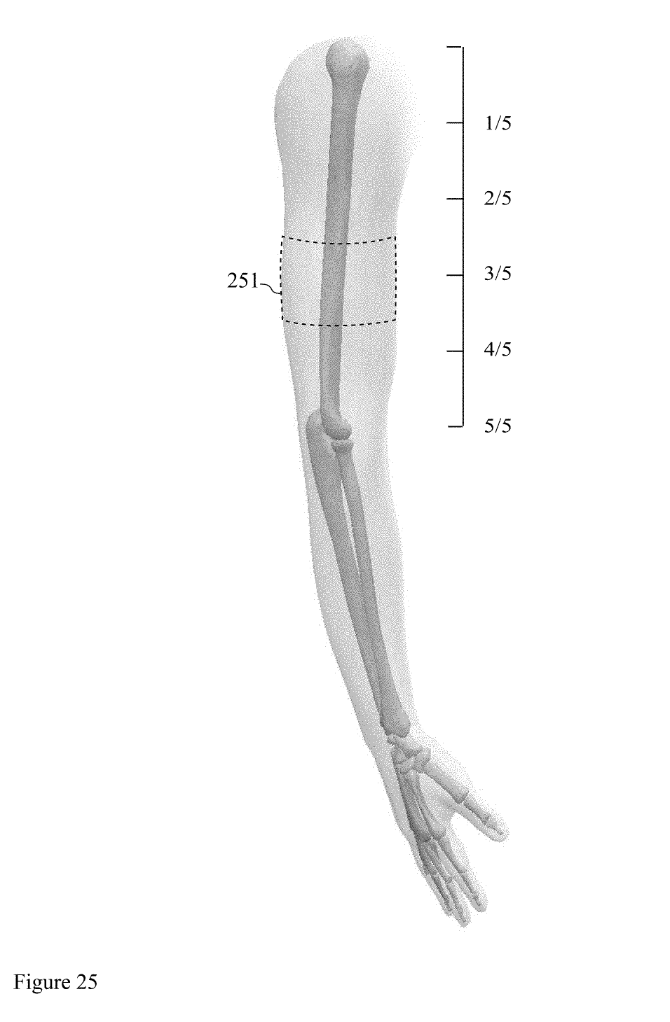

Particular locations for locating anatomic anchor points to position biomechanical sensors on patient's anatomy to monitor swelling in a patient's arm are shown in FIGS. 22-25. In an ulna or radius fracture, sensors can be located proximate the muscle of extensor (221) and flexor (222) muscles (FIG. 22). A target circumferential area is roughly located 2/5 length of the forearm (231) measuring from proximal end (FIG. 23). In a humerus fracture, sensors can be located proximate the muscles of triceps (241) and biceps (242) (FIG. 24). A target circumferential area is roughly located 3/5 length of the forearm (251), measuring from proximal end (FIG. 25).

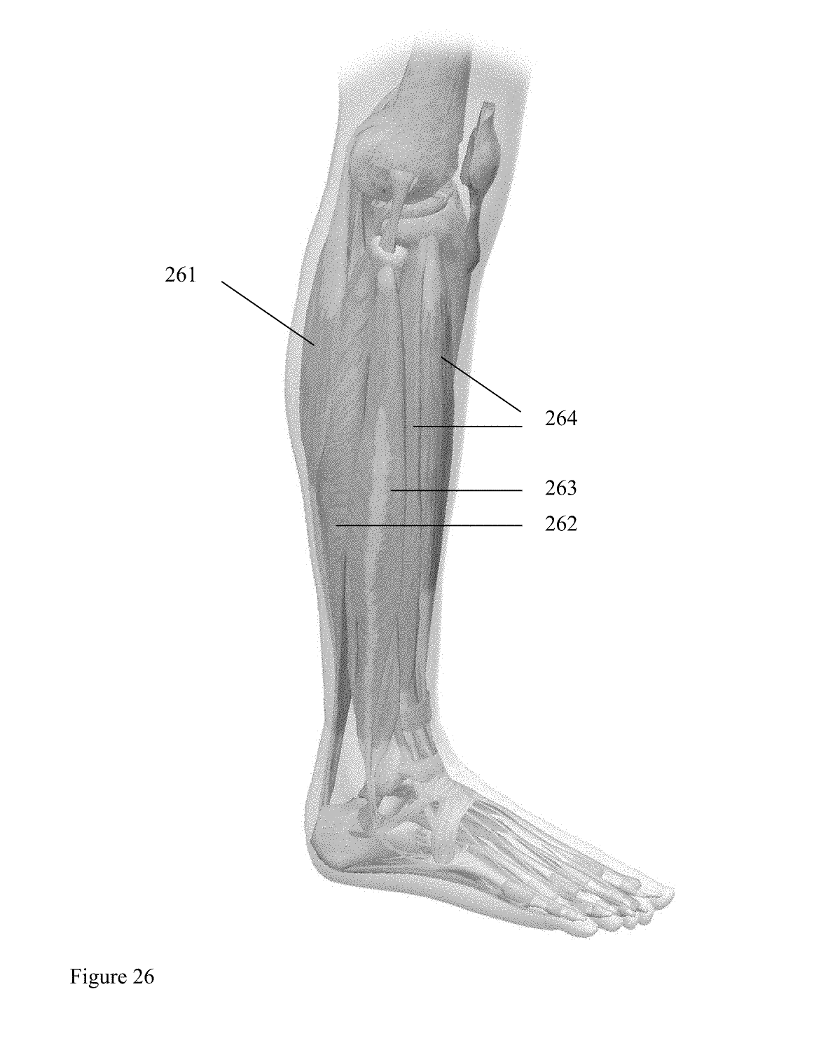

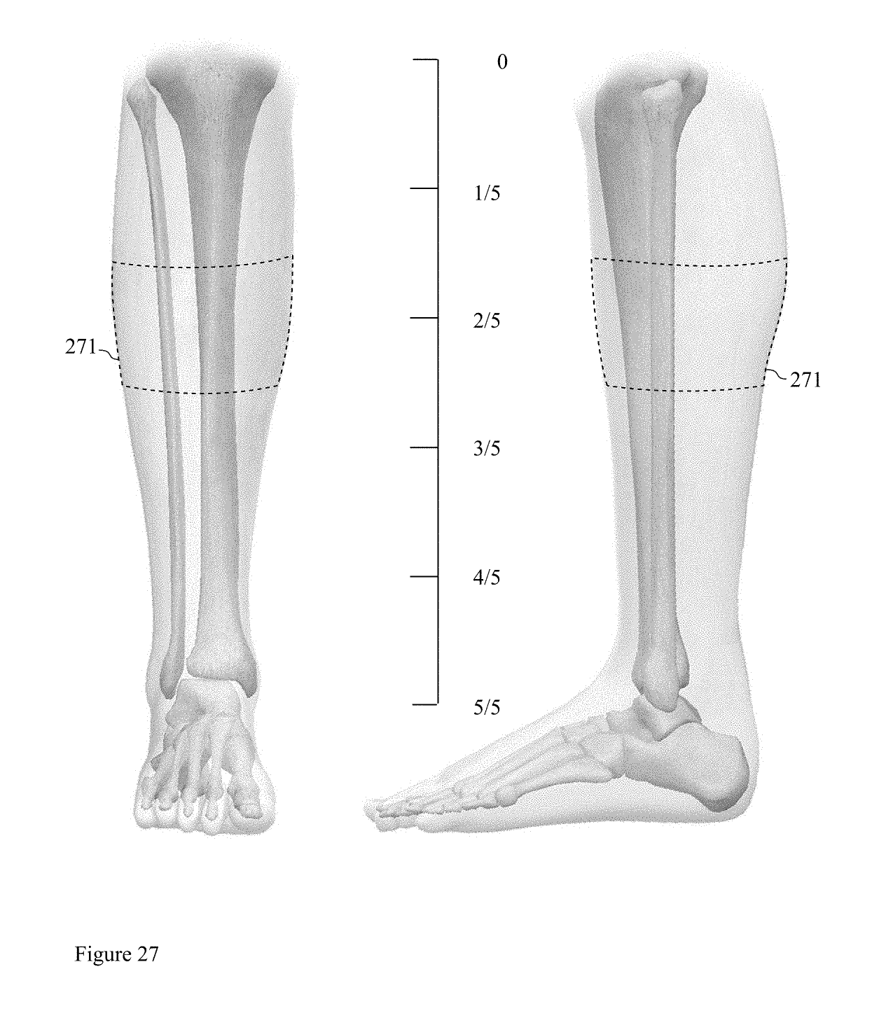

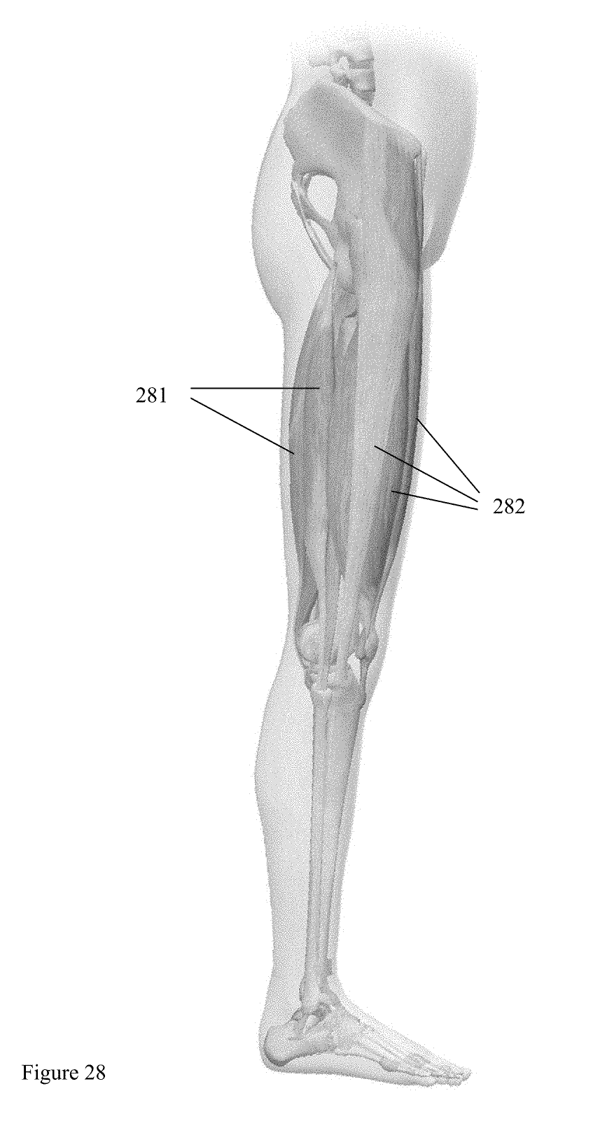

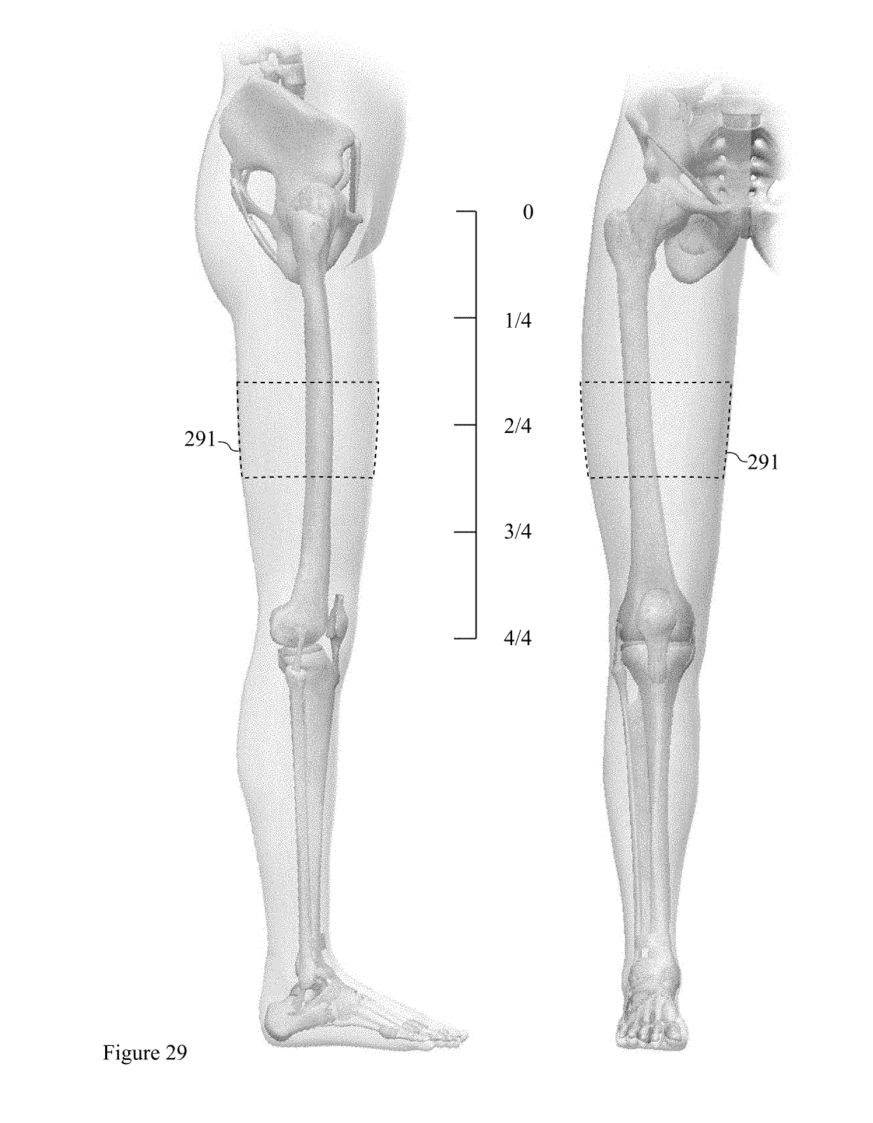

Referring to FIGS. 26-29, in fractures of the tibia or fibula, sensors can be located proximate the muscle of gastrocnemius (261), posterior compartment (262), lateral compartment (263) and anterior compartments (264) (FIG. 26). A target circumferential area it is roughly located 2/5 length of the lower leg (271), measuring from proximal end (FIG. 27). In femur fractures, sensors can be located proximate the muscle of the posterior compartments (281) and/or muscle of the anterior compartments (282) (FIG. 28). This circumferential area it is roughly located 2/4 length of the lower leg (291) measuring from proximal end (FIG. 29).

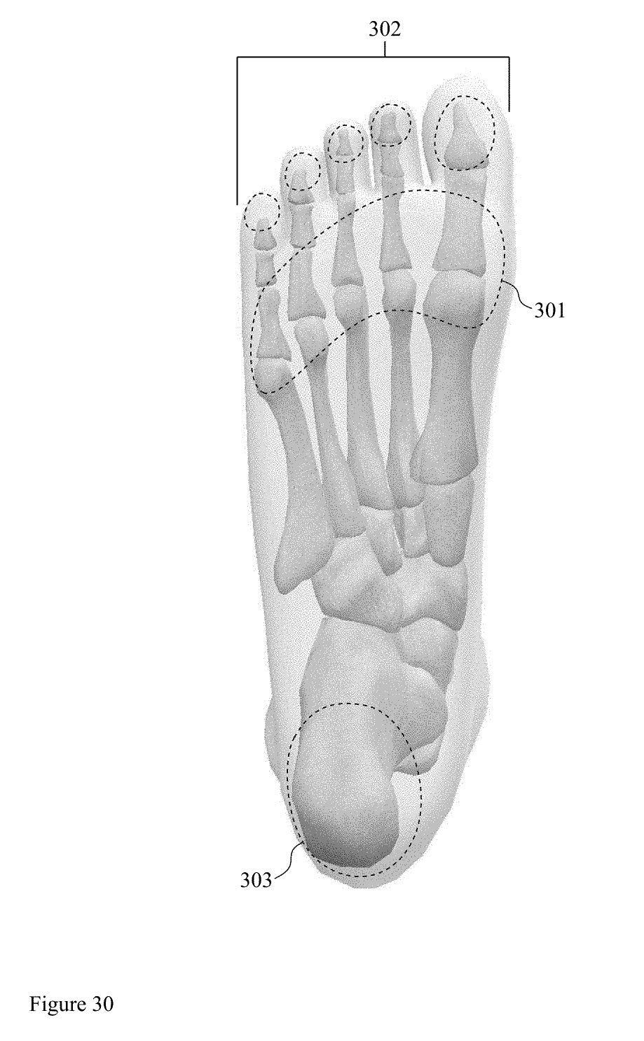

Patients with diabetes can develop many different foot problems. Conditions which are at first manageable can worsen and lead to serious complications. Foot problems are exacerbated when there is nerve damage referred to as neuropathy. Patients with diabetes can also suffer from special skin conditions because diabetes affects the capillaries, including thickening of skin resulting in calluses which limit the supply of skin nutrients skin. Callus formation occurs in high numbers of patients with diabetes, absent foot pulses, formation of hammer toe interphalangeal, and foot ulcers. A hammer toe occurs from a muscle and ligament imbalance around the toe joint which causes the middle joint of the toe to bend and become stuck in flexion. Callus and ulcers usually occur on the bottom of the foot monitoring pressure profile in these area provides diagnostic data for these cases. FIG. 30 illustrates the convenient anatomic locations on the foot to monitor the development of callus and ulcers which appear along the pad of the foot (301) (from the first to the fifth plantar metatarsophalangeal joints and plantar metatarsals), the bottom of the toes (302) (from the first to the fifth plantar distal phalanges), and the heel of foot (303) (plantar calcaneus). Monitoring pressure profile in these areas provides diagnostic data for patients suffering from diabetes.

Pressures resulting from changes in compressive force can be measured using c pressure sensors, including piezoresistive, piezoelectric, capacitive, and the like. The pressure sensors are placed in predetermined locations within in body scaffold. Usually. the pressure sensors will have padding structures to present a long term comfortable interface to patient anatomy, Pressure sensors to monitor pressure, data transmission device(s) for collecting, processing and transferring the data, batteries to power the device and wiring for internal data transfer will often also be incorporated into the body scaffolds of the present invention.

In addition to commercially available sensors, the systems of the present invention will often utilize custom pressure sensors designed to provide a more conformable or more effective body interface. Such custom sensors can also facilitate data transmission, provide more accurate data mining capabilities, be shaped to conform to specialized body scaffold geometries, and the like. Exemplary fabrication methods may utilize three-dimensional printed molds and microelectromechanical (MEMS) fabrication techniques to provide barometer chips with temperature sensors, instrumentation amplifiers, analog to digital converters, standard bus interface (an example sensor is a Miniature I2C Digital Barometer MPL115A2), and other specialized capabilities. Fabrication may also include pouring of viscoelastic materials (rubber, silicone) in vacuum environment in order to manufacture personalized biomechanical sensors for the task.

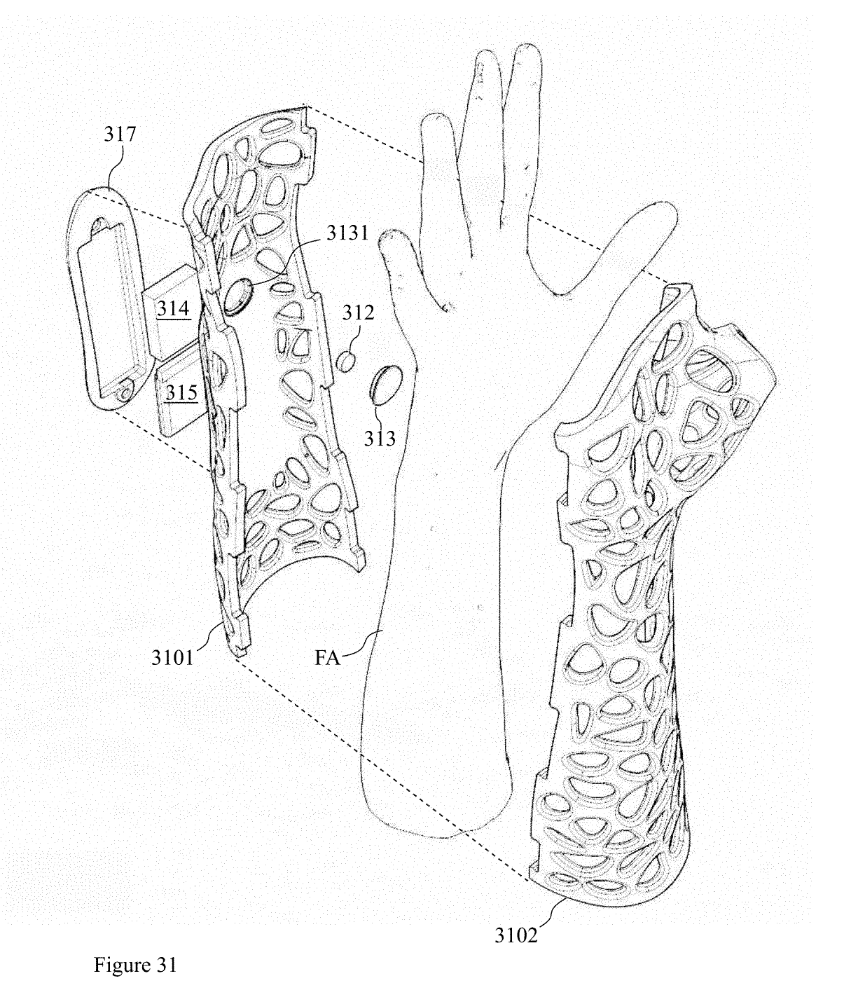

An example of flexion monitoring in the wrist area is illustrated in FIG. 31. A pressure sensor (312) having padding (313) is positioned in a receptacle (3131) in a shell 3101 of the body splint. The receptacle is positioned to located the pressure sensor (312) adjacent the posterior carpal as described previously with respect to FIG. 4. The full body scaffold or splint includes a second shell (3010) which attached to the first shell to form a splint which fully circumscribes the forearm FA. The wrist flexion monitoring system further incorporates a microcomputer (314) with relevant capabilities, batteries (315), and wiring, and optionally includes a cover (317) for the electronic components.

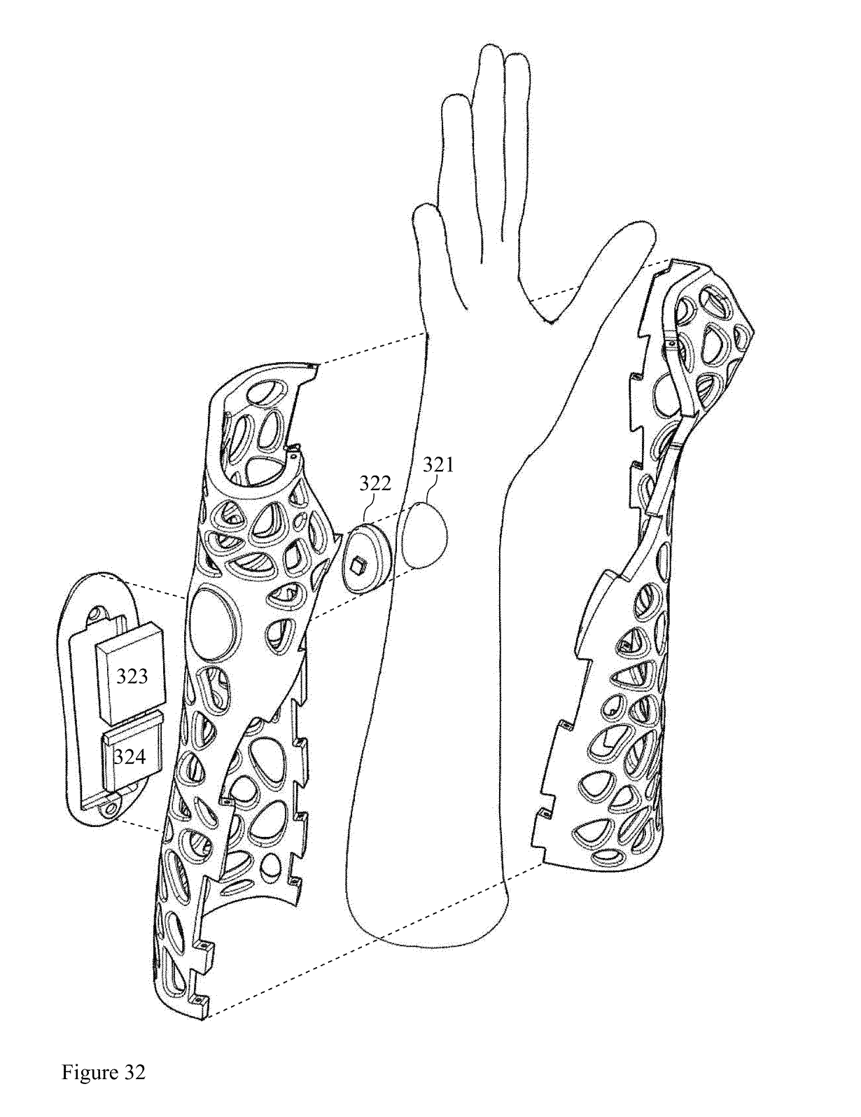

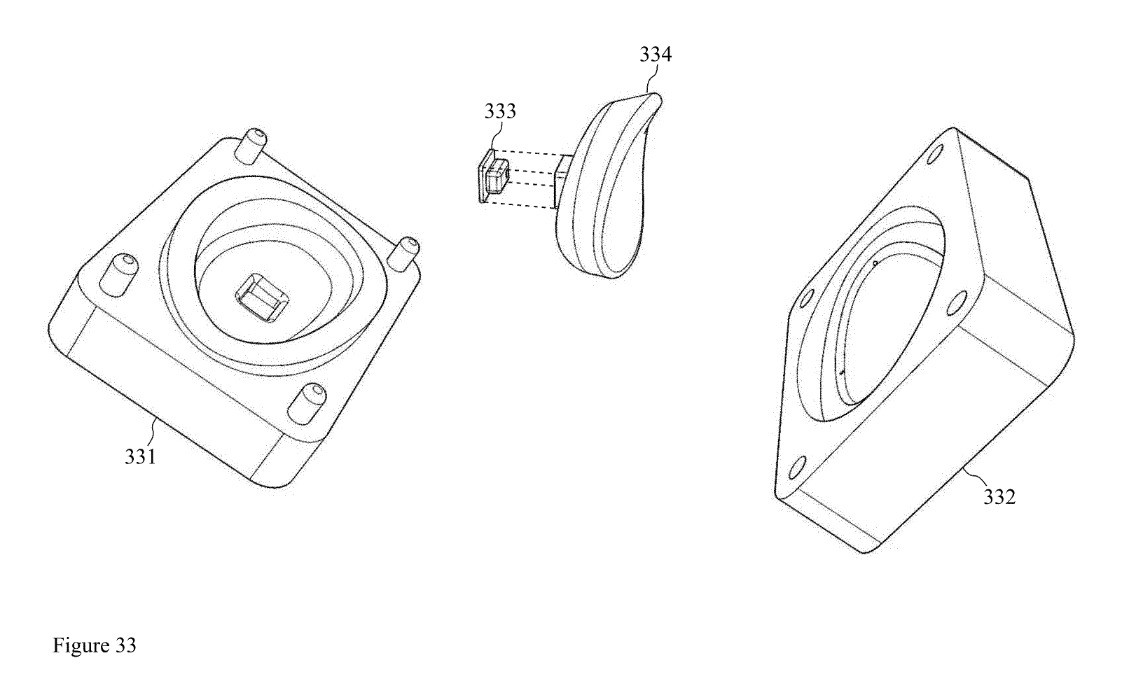

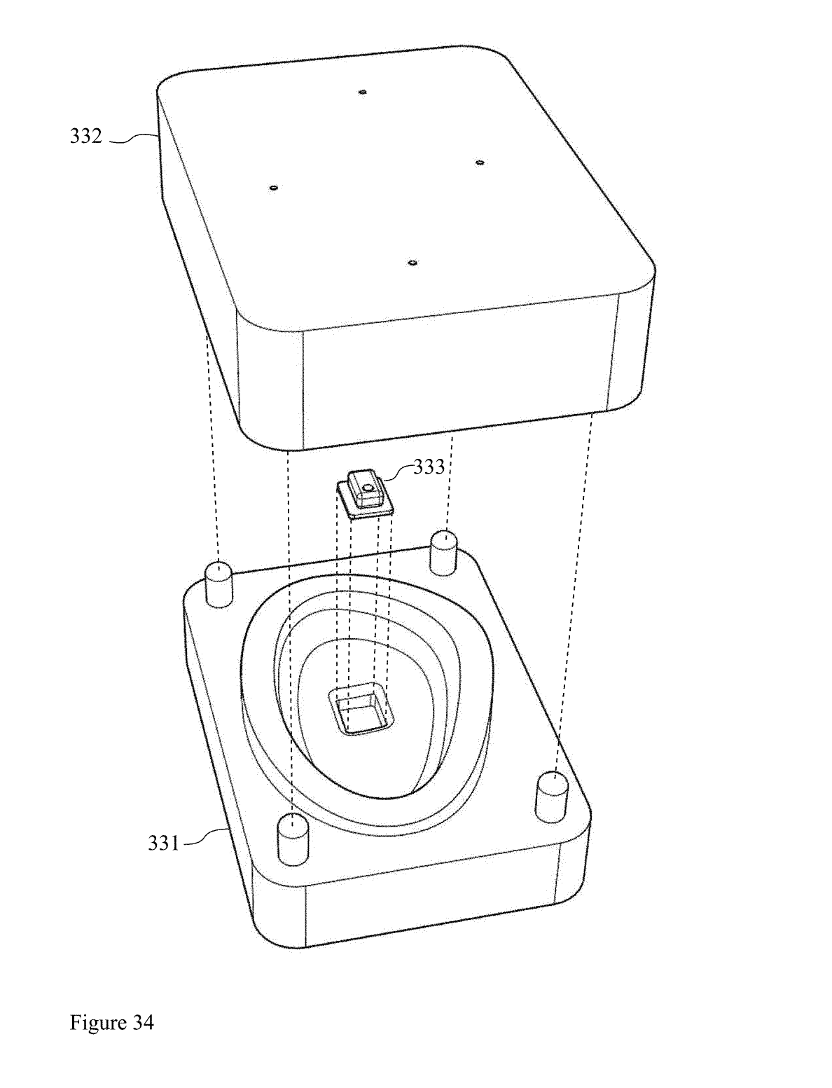

Manufacturing of a body splint for monitoring radial deviation of a wrist is described with reference to FIGS. 32-34. Step 1: The target lateral wrist area for tracking pressure is marked with (321). Step 2: During geometric data gathering and CAD planning stages, a three-dimensional surface geometry of the patient's lateral wrist is captured and used to design mold pieces (331 and 332 in FIGS. 33 and 34). An interior surface of the top mold (332) conforms to the wrist geometry and an interior surface of the bottom mold (331) defines a cavity for placement of a MEMS barometer and PCB board (333). Step 3: An upper surface of the mems digital barometer (333) is placed at the bottom of the mold mechanism, as illustrated in FIG. 34. Step 4: A rubber or silicone material having viscoelastic properties similar to human body is poured into the mold, and the resulting block (322), rubber or silicone forms a robust compliant contact surface which communicates surface contact pressure to the MEMS transducer. Vacuum is important to prevent air trapping inside mems barometers. Step 5: the block (322) is molded for time, pressure and temperature selected depending on the thickness and the elasticity of rubber or siliconee used. Custom sensors covering circular areas around joints or the entire inner surface of the personal three-dimensional printed splints can be manufactured with this technique.

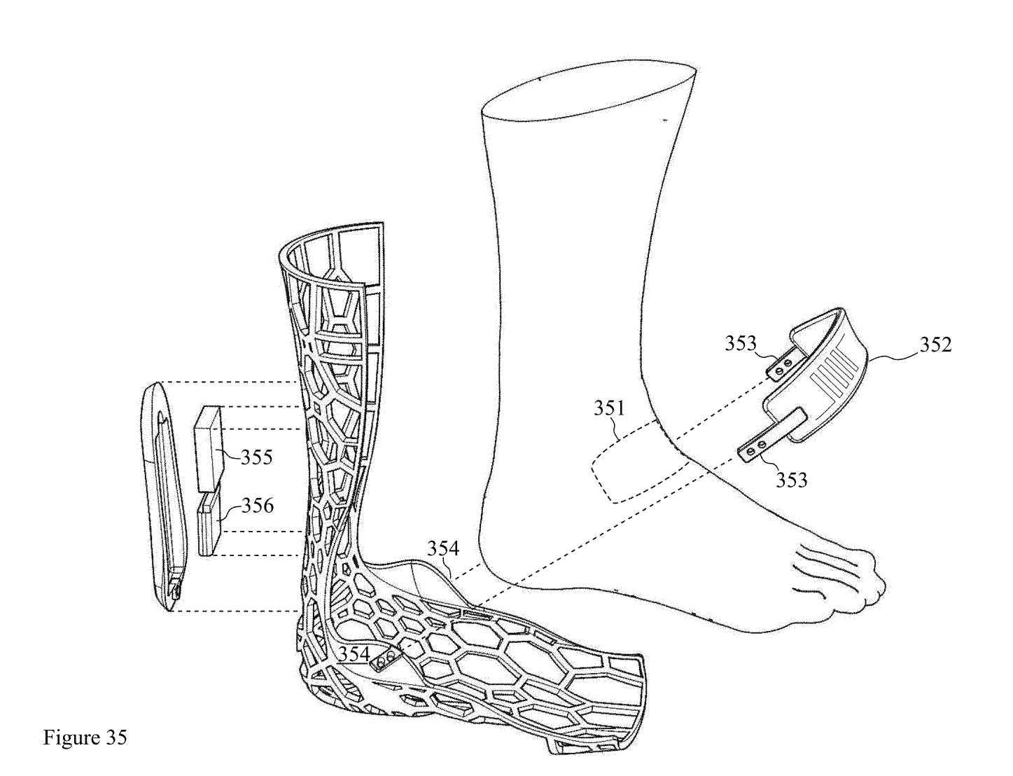

Tension measurement may be used as an alternative or in addition to pressure (compression) measurement. Tension and compression are opposites of each other and one can be converted to the other with ease. Any type of sensory technology monitoring tension can be used as a sensor in the present invention, e.g. strain gauges, tension monitoring fabrics, and the like. A strain gauge sensor typically utilizes changes in electrical conductance to monitor changes caused by tension on a flexible material surface. Although the target locations for pressure and tension measurement will generally be the same, the topology of the personal three-dimensional scaffold may be modified for particular purposes. An example is illustrated in FIG. 35 for plantar flexion monitoring in ankle area (as described above with respect to FIG. 11). A strain gauge sensor (352) (or a fabric with an embedded strain gauge sensor) is placed over the anterior tarsals region (351). The strain gauge sensor (352) includes additional snapping mechanisms (353) on each side to attach to the main body (354) of the personal three-dimensional printed splint (354). The system also incorporates a microcomputer (355) with relevant capabilities, batteries (356), and wiring. In practical application plantar flexion attempt creates a pressure profile in anterior tarsal area. This pressure is absorbed by the strain gauge sensor and cause elastic deformation in the structure, this elastic deformation is monitored by the changes in electro conductivity of the strain gauge sensor. Additional temperature data is also useful to track changes in elasticity of structures involved and further calibration.

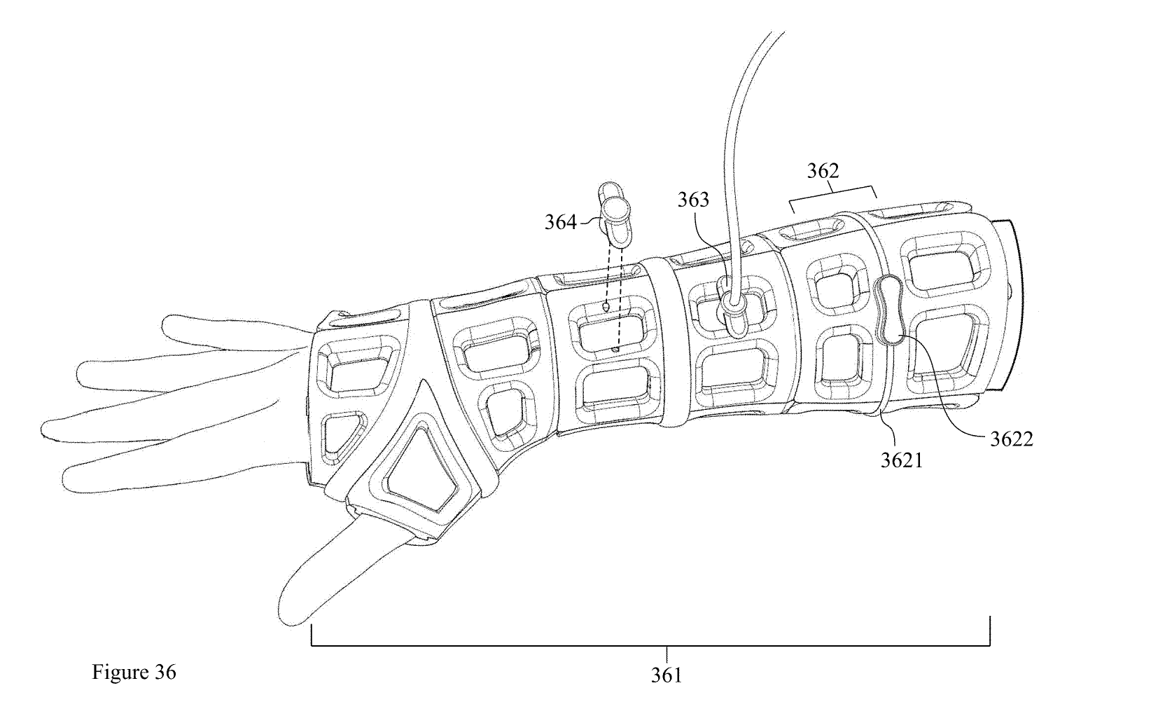

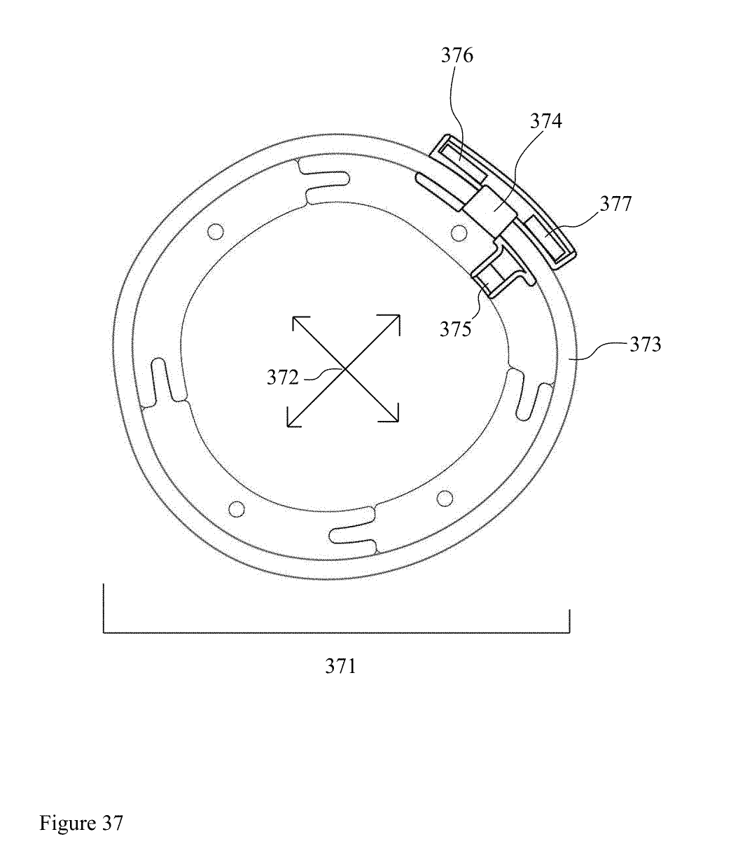

The body scaffolds of the present invention may also comprise adaptive or dynamic orthoses as an alternative to the static orthosis solutions that have been described to this point. Referring to FIGS. 36 and 37, an adaptive body scaffold is one that includes loins, hinges, articulation elements, and other dynamic features that allows the scaffold to reconfigure in response to changes in the body shape. The diagnostic system can use an adaptive splint's dynamic capabilities to monitor circumferential expansion through tension monitoring.

An upper extremity adaptive splint (361) includes sensory technology incorporating a circumferential expansion monitoring structure (362). The structure includes a rubber O-ring 3621 and a strain gauge 3622 can measure changes in electro-conductivity of the rubber O-ring due to stretching (strain gauge). Modular or embedded probes (effectors) and equipment strategically positioned to stimulate or deliver other therapies to the anatomic systems may also be provided (363, 364). Referring to FIG. 37, an alternative circumferential tension or swelling sensor (371) comprises an electrically conductive elastic band (373) is fixed to a stress sensor (e.g. a volt-ohm meter) (374). In this example the splinted area extends from the metacarpal phalangeal joints to proximal ulna and radius. The inflammatory monitoring system is located on muscle belly of extensor and flexor muscles. (375) is a temperature sensor monitoring the local body temperature of the patient, this data is also important since edema accumulation presents itself with an increased body temperature. The adaptive orthotic structure (371) is held together by pressure applied to the rubber O-ring (373). In the event of swelling, internal pressure will cause the rubber O-ring to expand cause changes in the electro conductivity of the o ring. In most cases edema presents itself with heat, item marked with 375 is a temperature sensor for monitoring the local body temperatures of the patient. The system also incorporates a microcomputer with relevant capabilities (376), batteries (377), and wiring.

The body scaffolds of the present invention may be used with external structures to deliver force to the body surface to in turn cause motion, tension, compression, and torque in the splinted anatomic structures. Diagnostic system generate and collect data representative of the biomechanical process of range of motion; flexion, extension, deviation, rotation, pronation, supination and other single or multi-axis motion in correlation with the external force applied through the orthotic structure. Restrained or incipient motion means that the external force applied by the scaffold is greater then the resistive internal force(s) until equilibrium is reached.

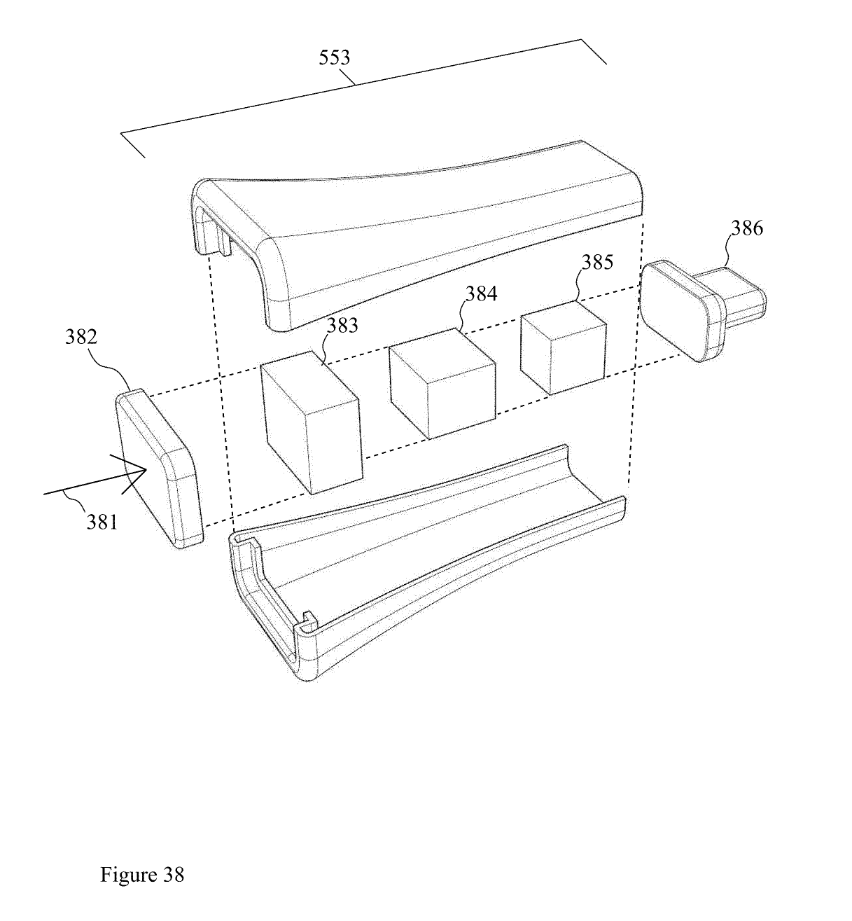

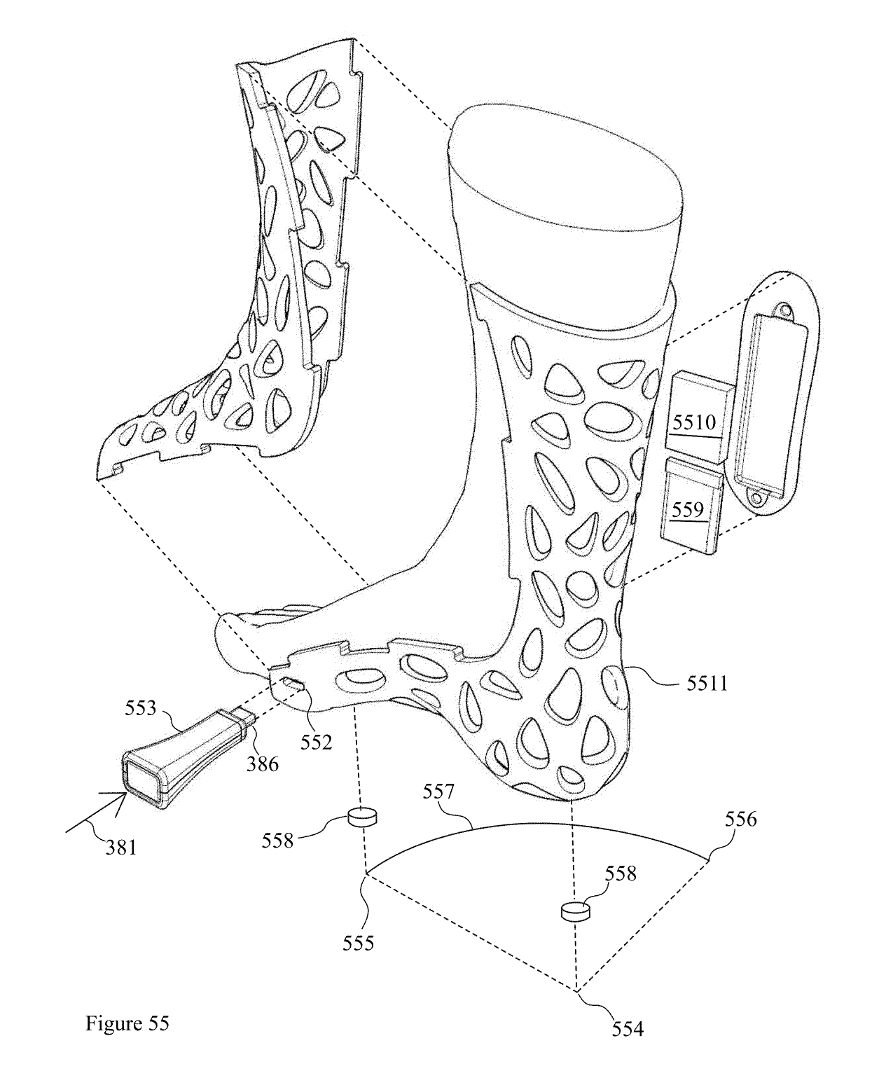

Referring to FIGS. 38 and 55, the system may digitize this evaluation by introducing a novel modular sensory probe in to the process (553). A force (381) is applied to the pressure sensor 382 located on the diagnostic probe (553). This force eventually causes rotational motion to the splinted anatomic structure (557). The change in three dimensional space is tracked with gyroscopes and accelerometers (383) embedded inside the diagnostic probe. The system also incorporates a microcomputer (384) with relevant capabilities, batteries (385), and wiring to perform the task. The diagnostic probe is a modular tool which can be attached to a personal three-dimensional printed splint (5511) through a socket (386) (alternatively, magnetic systems can also be used). A counter socket (552) is located on a relevant location in the personal three-dimensional printed splint.

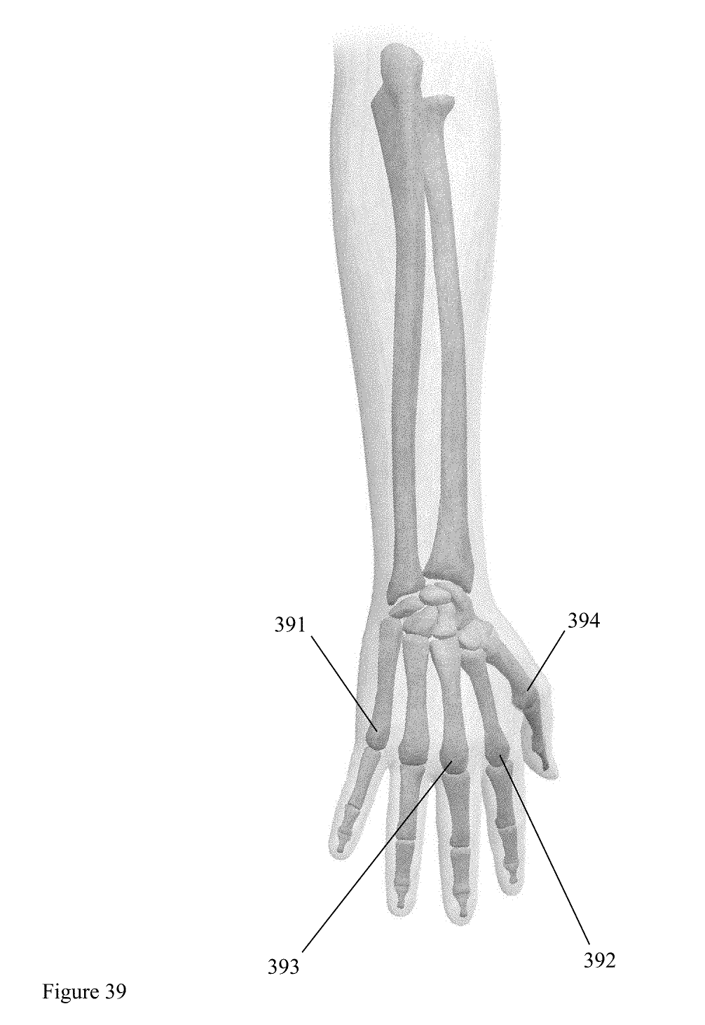

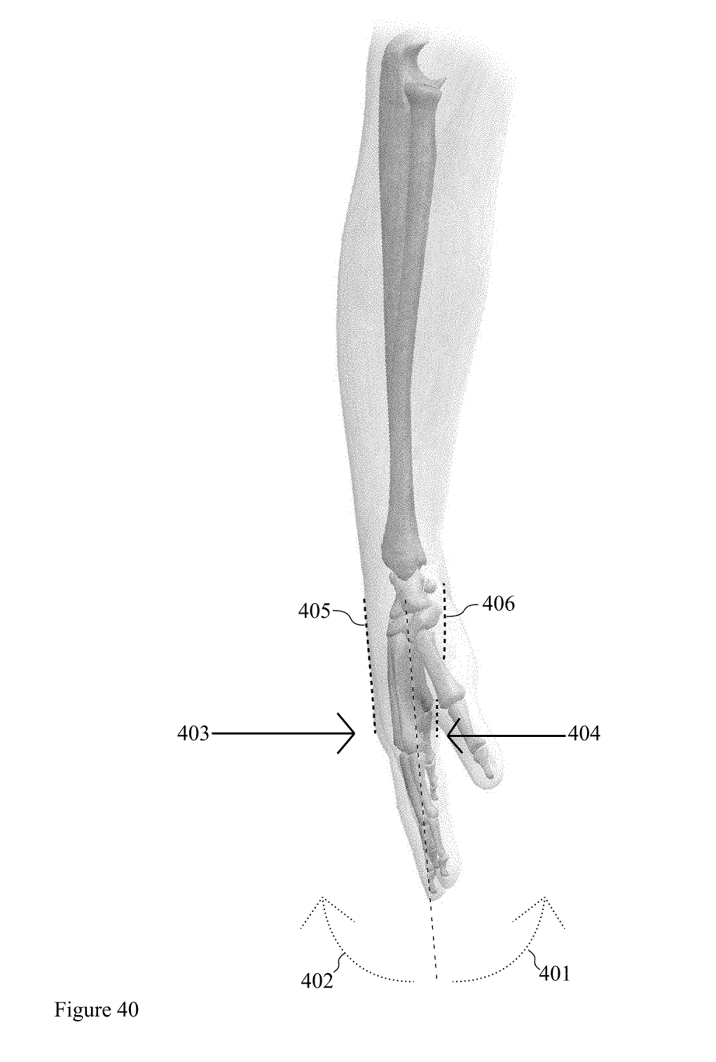

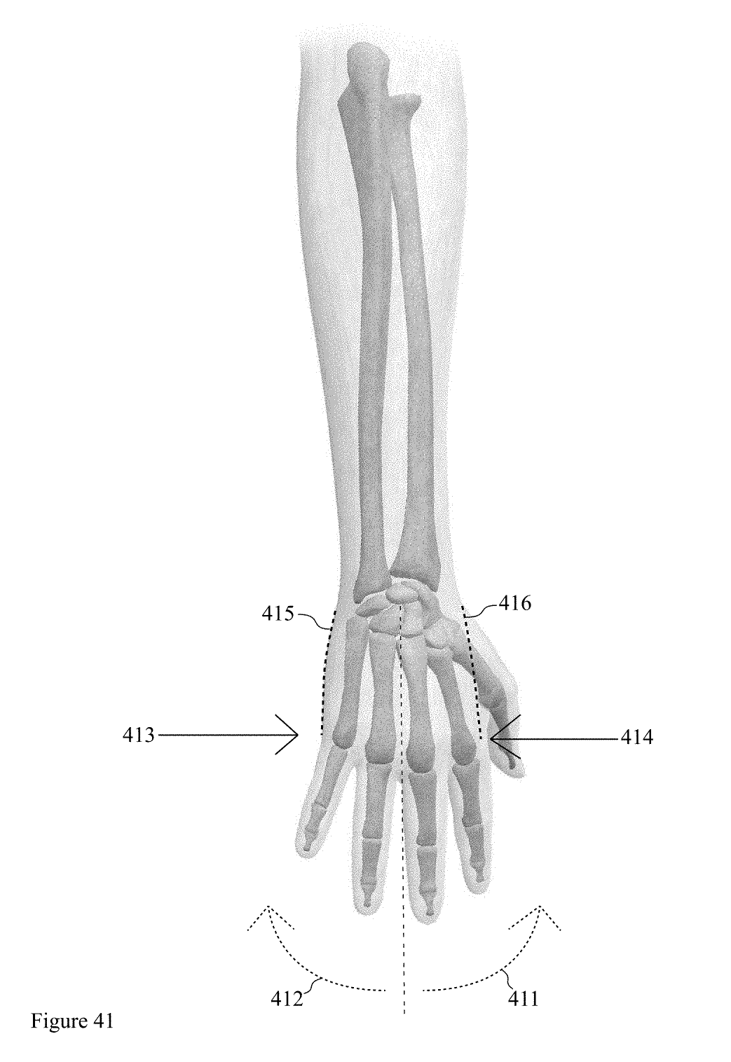

Referring to FIGS. 39-41, rotation may be induced to an upper extremity joint by applying a force perpendicular to the body scaffold or splint (also to the anatomic structure underneath) and lying within or parallel to the intended motion plane of the joint. The joint is subjected to this rotational force trough lever arm principle. Any point on a straight line starting from the joint to the distal end of the scaffold (located on the relevant motion plane of the intended motion and applied perpendicularly) is convenient to obtain the desired data. Referring to FIG. 40, flexion in the sagittal plane (401) can be induced by applying a force (403) during the orthopedic evaluation. The force can be delivered to a region (405) extending from posterior carpals to posterior third metacarpophalangeal region. Extension (402) is induced by applying force (404) to a region (406) extending from the anterior carpals to the anterior third metacarpophalangeal region. Referring to FIG. 41, radial deviation (411) in the frontal plane is achieved by applying a force (413) during orthopedic evaluation. The force may be delivered in region (415) extending from the lateral carpals to lateral fifth metacarpophalangeal region. Ulnar deviation (412) in the frontal plane is achieved by applying a force (414) during orthopedic evaluation; 414 is the demonstration of the force applied. The force may be delivered in region (416) extending from the medial carpals to medial second metacarpophalangeal region.

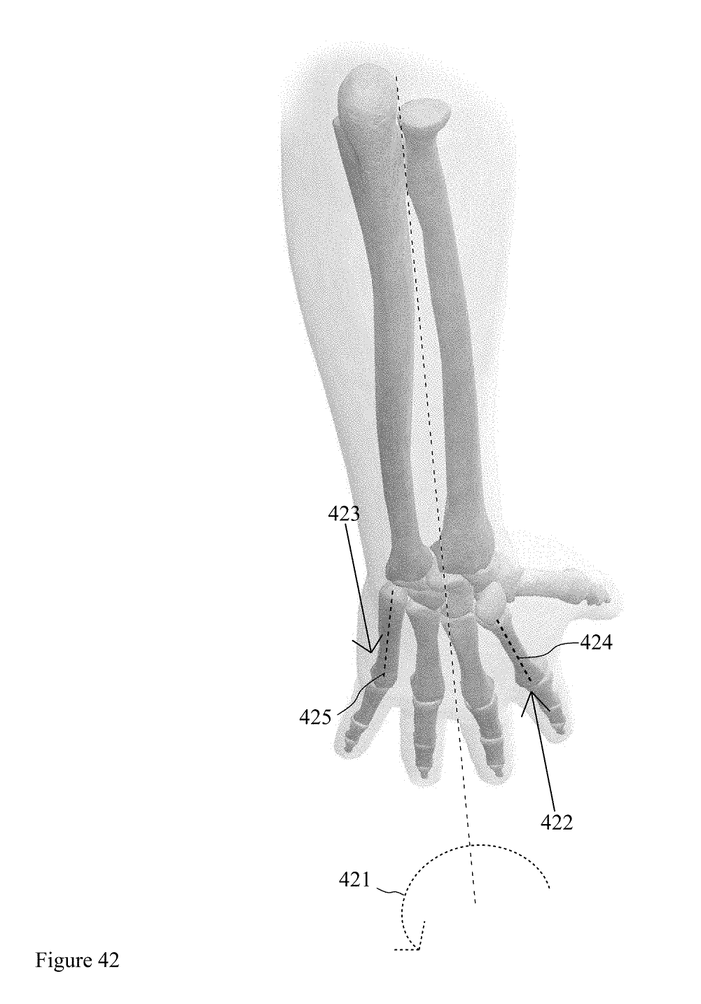

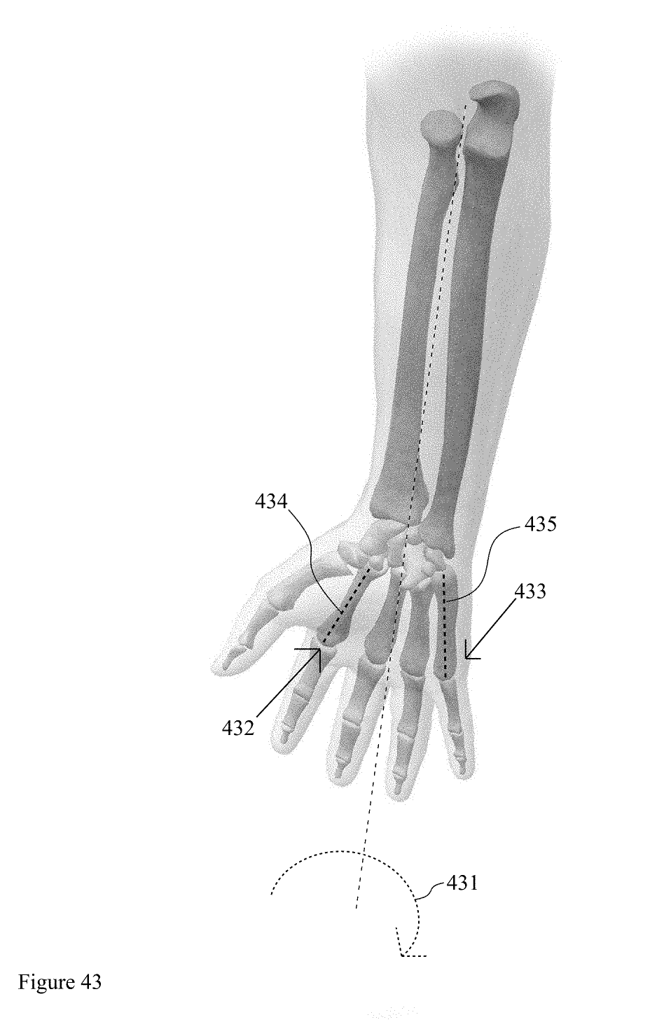

Referring to FIG. 42, wrist pronation (421) is a rotational motion in the transverse plane and is induced by forces (422 and 423) delivered to two anatomic locations (424 and 425). The most convenient anatomic locations to deliver intended motion are posterior second metacarpophalangeal region (424) and the anterior fifth metacarpophalangeal region (425). Referring to FIG. 43, wrist supination (431) is also a rotational motion in the transverse plane and is induced by forces (432 and 433) delivered to two anatomic locations (434 and 435). The most convenient anatomic locations to deliver the forces are the anterior second metacarpophalangeal region (434) and the posterior fifth metacarpophalangeal region (435).

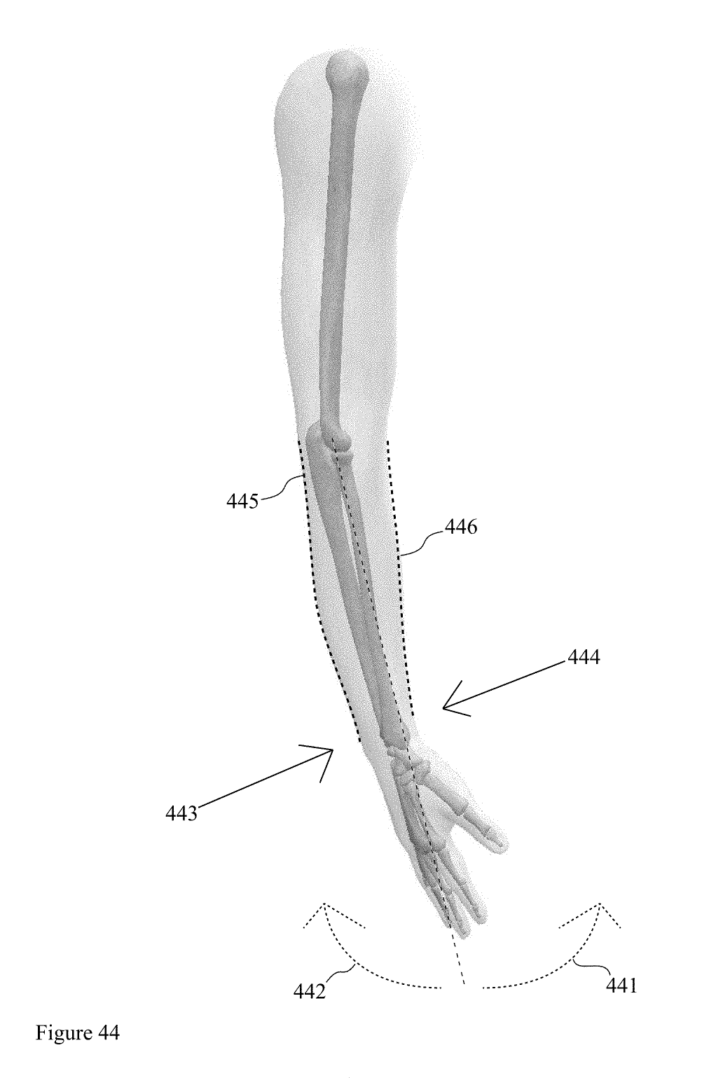

Referring to FIG. 44, elbow flexion (441) in the sagittal plane may be induced by applying a force (443) to a region (445) extending from lateral elbow to lateral carpals region (or depending on the distal end of the splint, lateral fifth metacarpophalangeal). Elbow extension (442) in the sagittal plane may be induced by applying a force (444) to a region (446) extending from the medial elbow to medial carpals region (or depending on the distal end of the splint, lateral second metacarpophalangeal).

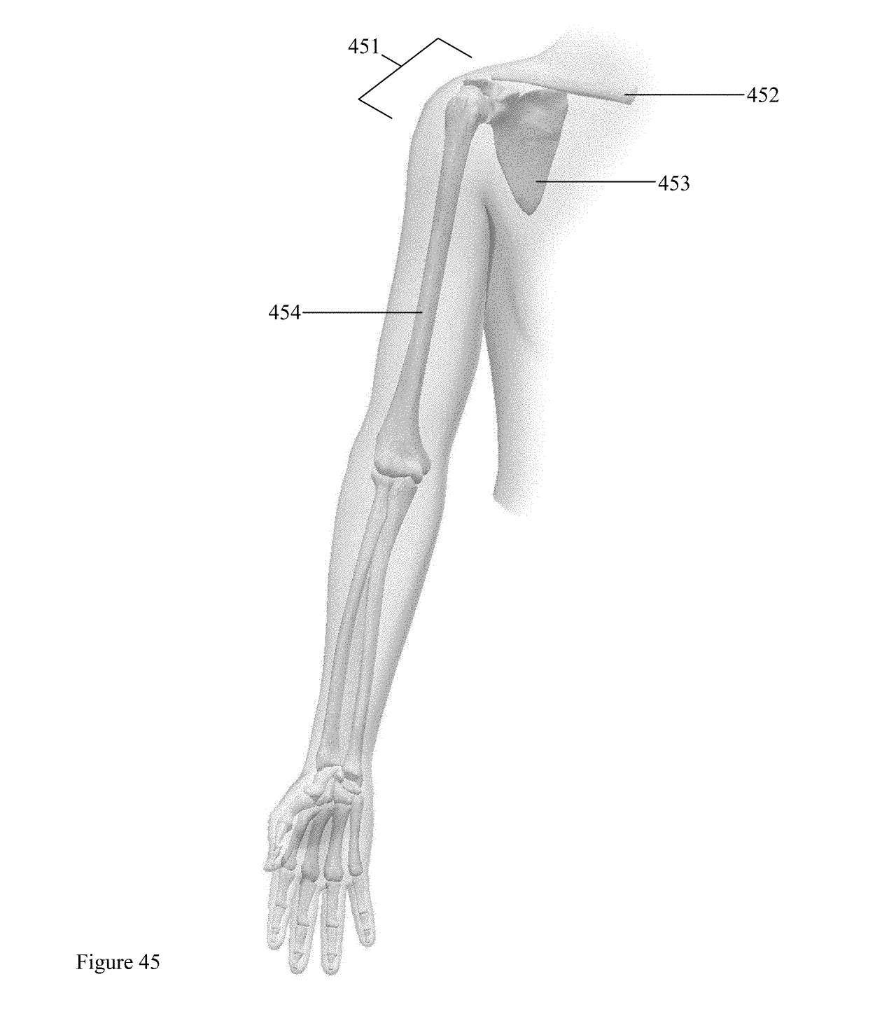

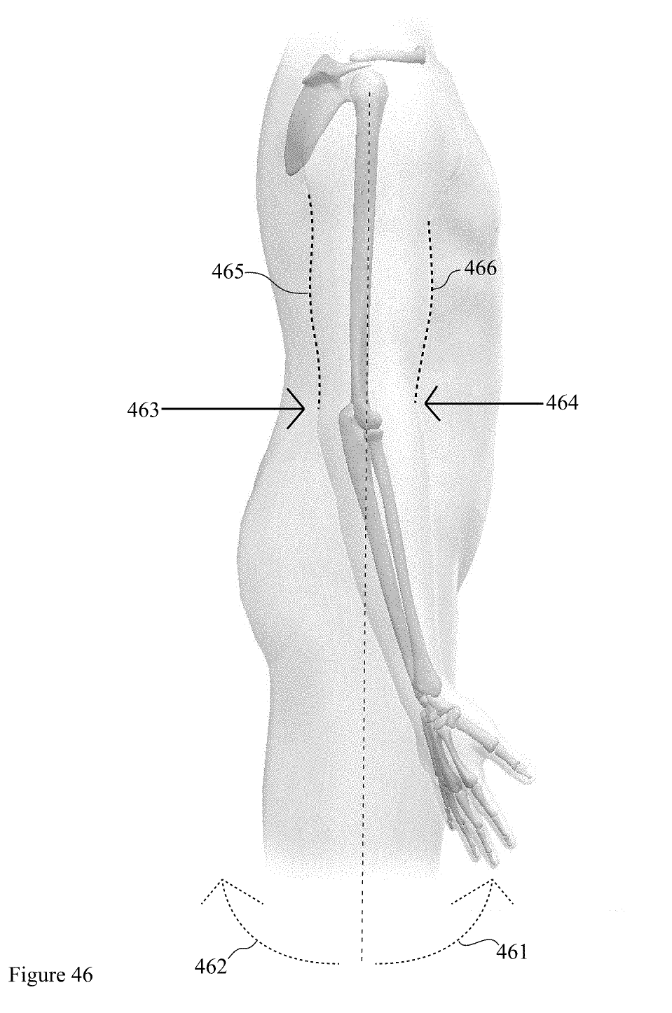

Referring to FIGS. 45-47, the shoulder (451) is joint connecting the arm with the torso. The shoulder is made up of three bones: the clavicle (452), the scapula (453) and the humerus (454). As shown in FIG. 46, shoulder flexion (461) in the sagittal plane is induced by applying a force (463) to region (466) extending from the posterior proximal upper arm to the posterior elbow region (posterior humerus region). Shoulder extension (462) in the sagittal plane is induced by applying a force (464) to a region (466) extending from the anterior proximal upper arm to anterior elbow (anterior humerus region).

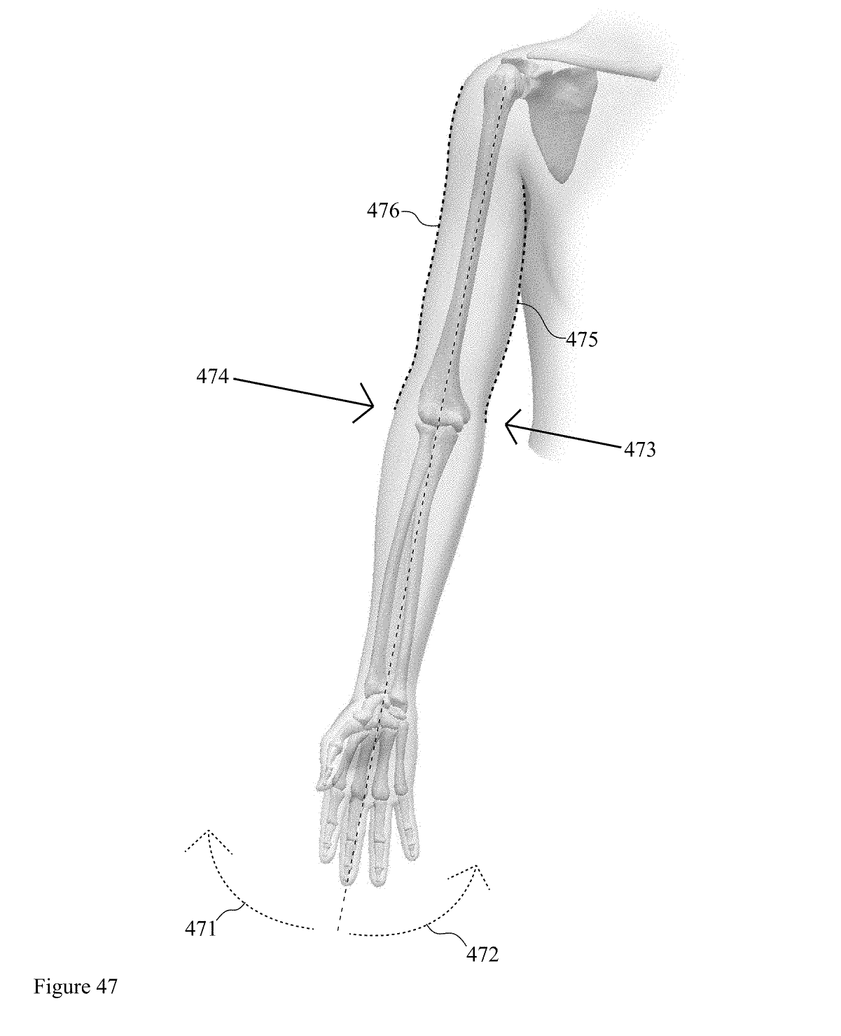

As shown in FIG. 47, shoulder abduction (471) in the frontal plane is induced by applying a force (473) to a region (475) extending from the medial proximal upper arm to the medial elbow (anterior humerus region). Shoulder adduction (472) in the frontal plane is induced by applying a force (474) to a region (476) extending from the lateral proximal upper arm to the lateral elbow region (lateral humerus region). Medial and lateral rotation of humerus requires the upper extremity to be in flexion position (preferably 90 degrees). The anatomic locations and force dynamics of this orthopedic evaluation are applied from elbow flexion and extension.

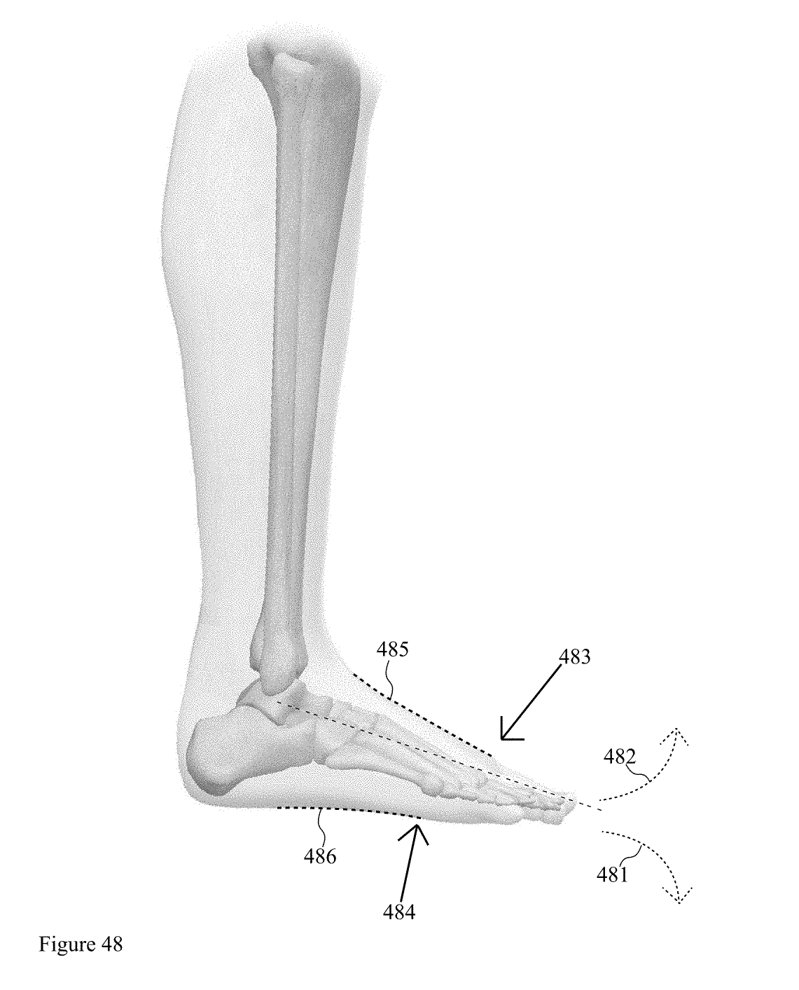

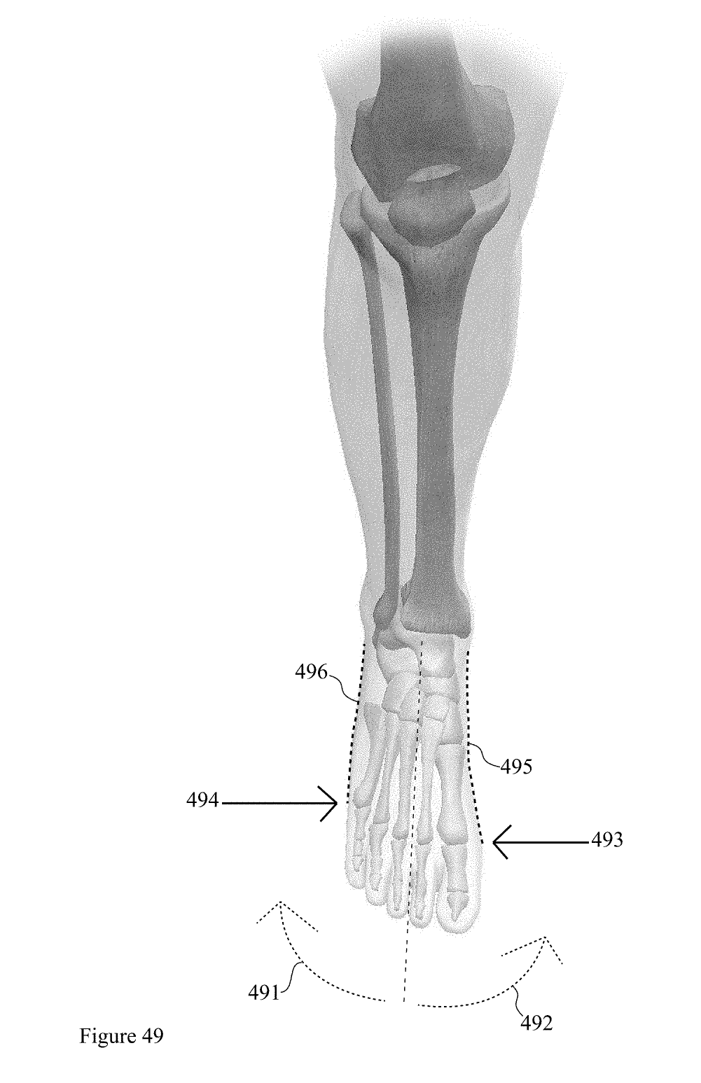

Referring to FIGS. 48-49, ankle plantar flexion in sagittal plane (481) is induced by applying a force (483) to a region (485) extending from the posterior tarsals to the posterior the metatarsophalangeal region. Doris flexion in the sagittal plane (482) is induced by applying a force (484) to a region (485) that extends from the anterior tarsals to the anterior third metatarsophalangeal region. As shown in FIG. 49, ankle abduction in the transverse plane (491) is induced by applying a force (493) to a region (495) extending from the medial tarsal to medial first metatarsophalangeal region. Ankle adduction in transverse plane (492) is induced by applying a force (494) to a region (496) extending from the lateral tarsal to the lateral fifth metatarsophalangeal region.

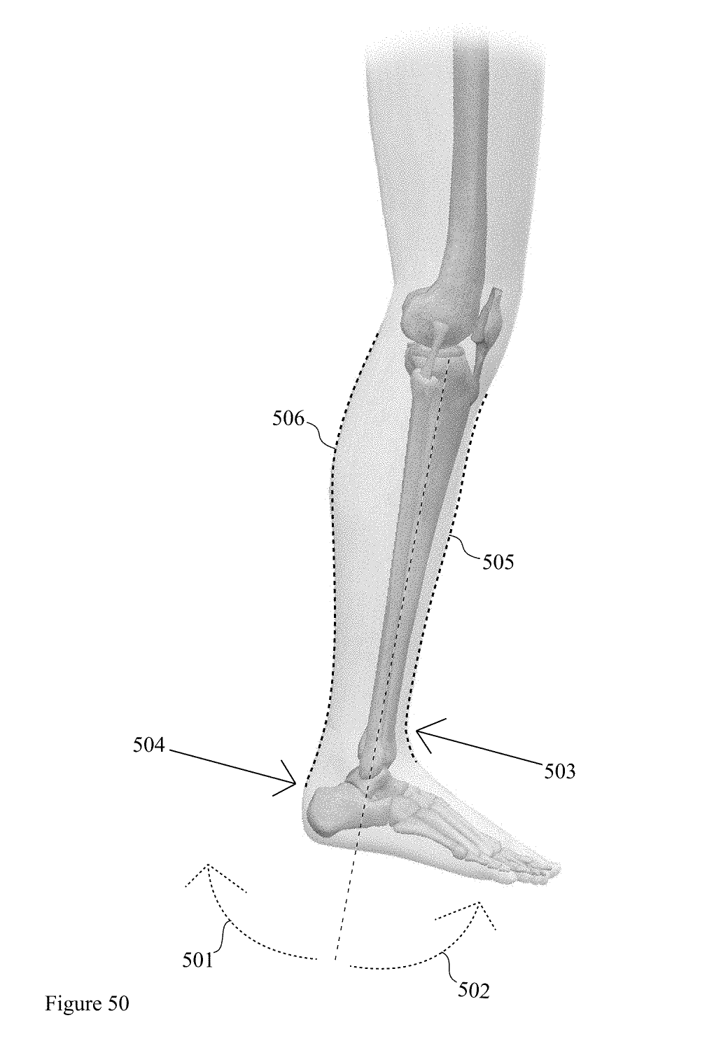

Referring to FIG. 50, knee flexion in sagittal plane (501) is induced by applying a force (503) to a region (505) extending from the anterior knee to the anterior tibia region. Knee extension in sagittal plane (502) is induced by applying a force (504) to a region (506) extending from the posterior knee to the posterior tarsal.

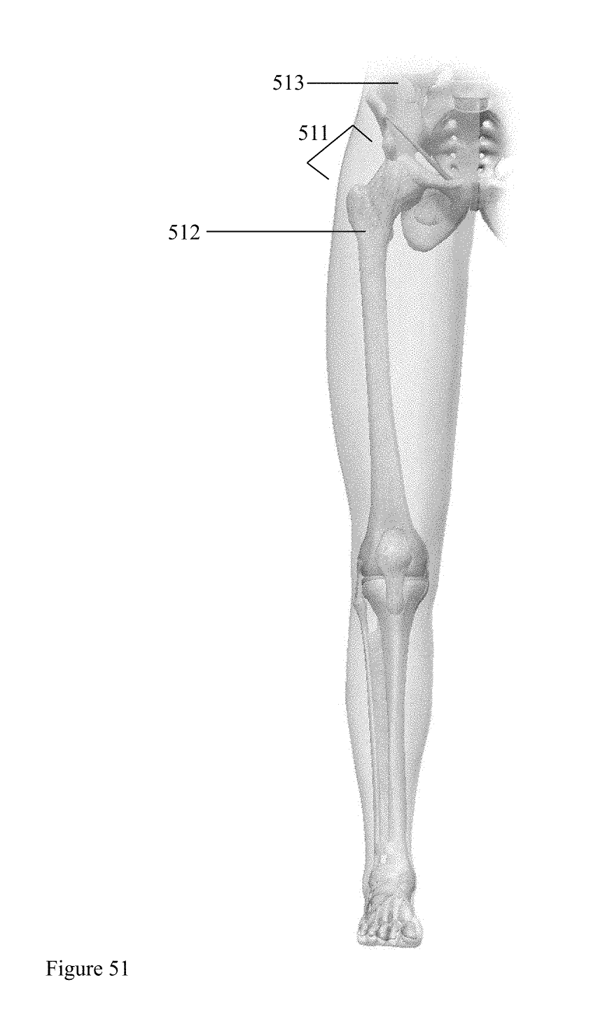

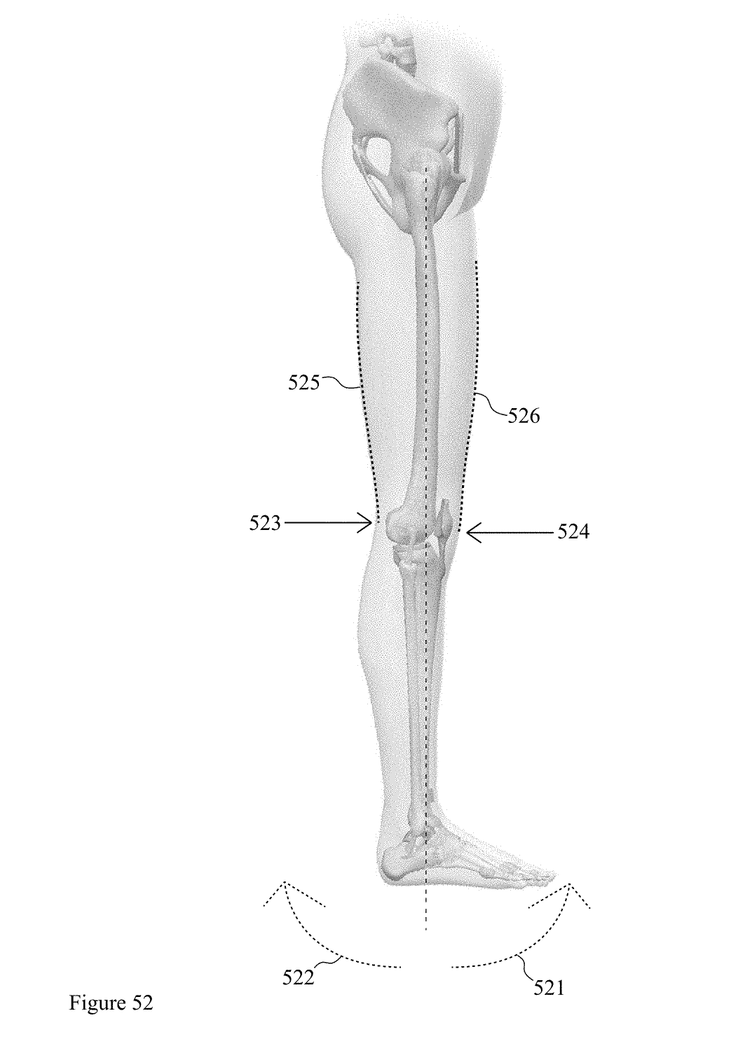

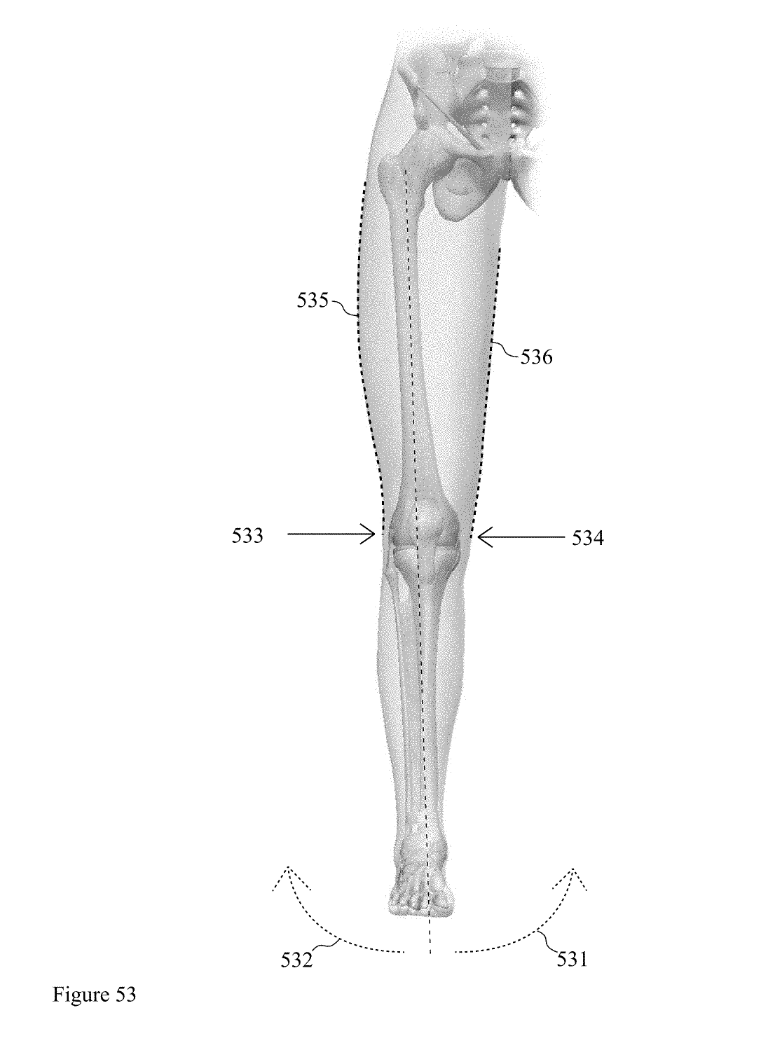

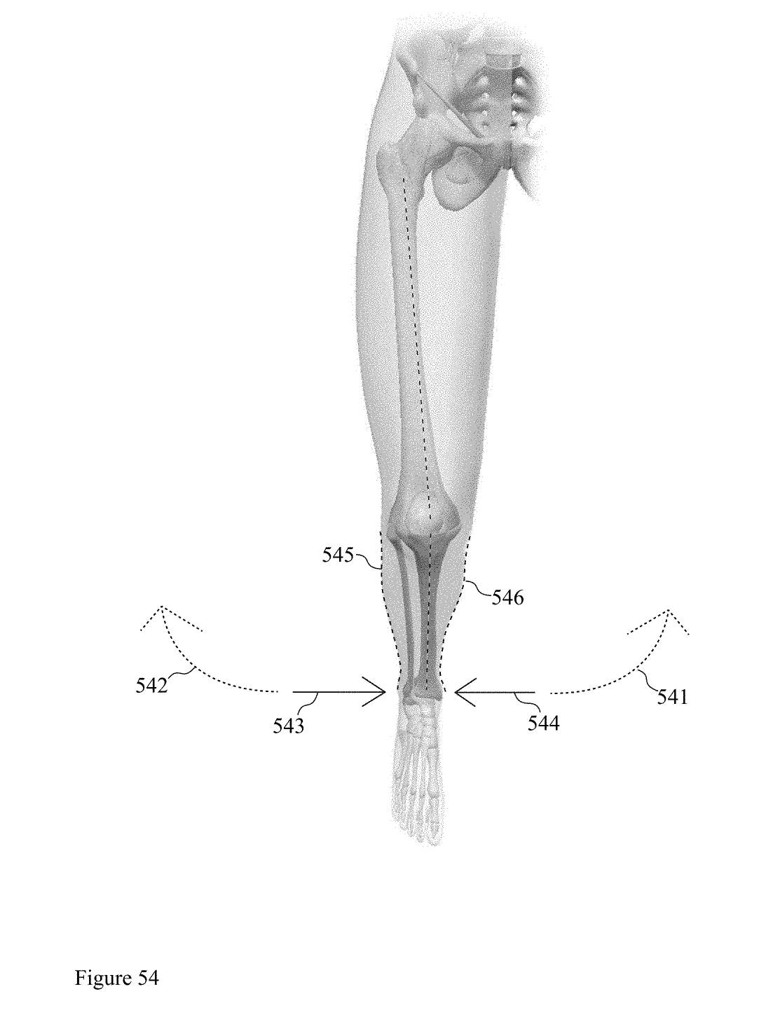

Referring to FIGS. 51-54, the acetabulofemoral (hip) joint is the joint between the proximal femur (512) and the pelvis (513) (acetabulum). The leg is the entire lower extremity (femur, knee, tibia, fibula, ankle and foot. Hip joint flexion in the sagittal plane (521) is induced by applying a force (523) to a region (525) extending from the posterior hip joint to the posterior knee region (or depending on the distal end of the splint, posterior tarsal region). Hip joint extension in the sagittal plane (522) is induced by applying a force (524) to a region (526) extending from the anterior hip joint to the anterior knee (or depending on the distal end of the splint, anterior tarsal region). As shown in FIG. 53, hip abduction in frontal plane (531) is induced by applying a force (533) to a region (535) extending from the medial hip joint to the medial knee region (or depending on the distal end of the splint, medial tarsal region). Hip adduction in frontal plane (532) is induced by applying a force (534) to a region (536) extending from the lateral hip joint to the lateral knee region (or depending on the distal end of the splint, lateral tarsal region). As shown in FIG. 54, hip medial rotation in the transverse plane (541) (for this analysis the knee is must be in flexion preferably 90 degrees) in induced by applying a force (543) to region (545) extending from the lateral knee to the lateral tarsal region. Hip lateral rotation in the transverse plane (542) is induced by applying a force (544) to a region (546) extending from the medial knee to the medial tarsal. The diagnostic system provides attachable multiple sensory interface to these regions for relevant data collection and transmission.

The diagnostic and therapeutic systems of the present invention will frequently use embedded sensors and therapeutic elements as described and illustrated above. Additionally and alternatively, certain embodiments of the present may use and incorporate the diagnostic probe (553) previously described with reference to FIG. 38. The probe 553 is designed to be a tool to measure the force (381) delivered by the orthopedist and measure changes in three-dimensional space caused by anatomic motion in tandem with the applied force. The probe incorporates gyroscopes (383), accelerometers (384), a pressure sensor (381) and other related electronic equipment to perform the task (385). An example with lower extremity splints in range of motion evaluation for the ankle in transverse plane for abduction. The biomechanics foot abduction is covered in FIG. 49 and area marked with (495) is the pre-determined anatomic location for this data mining challenge. In FIG. 55, probe (551) provides the force applied by the orthopedic examiner. This force is transferred to personal splint (5511) through the diagnostic probe (553). Socket (552) ensures that the probe (553) is positioned 90 degrees to the patient anatomy. The diagnostic probe transfers the applied pressure to the splint. Central axis (554) is the axis of the rotation, the position of the foot before the orthopedic examination is marked with (555), and the maximum rotational position of the foot is marked with (556). Arch (557) is the range of motion curve for this particular examination

Alternatively gyroscopes and accelerometers (558) can be positioned on the orthotic for monitoring the changes in space but the anatomic location and the direction of the force applied must be preserved and assured embedding gyroscopes (558) and accelerometers (558) will require additional relevant device (559) and batteries (5510).

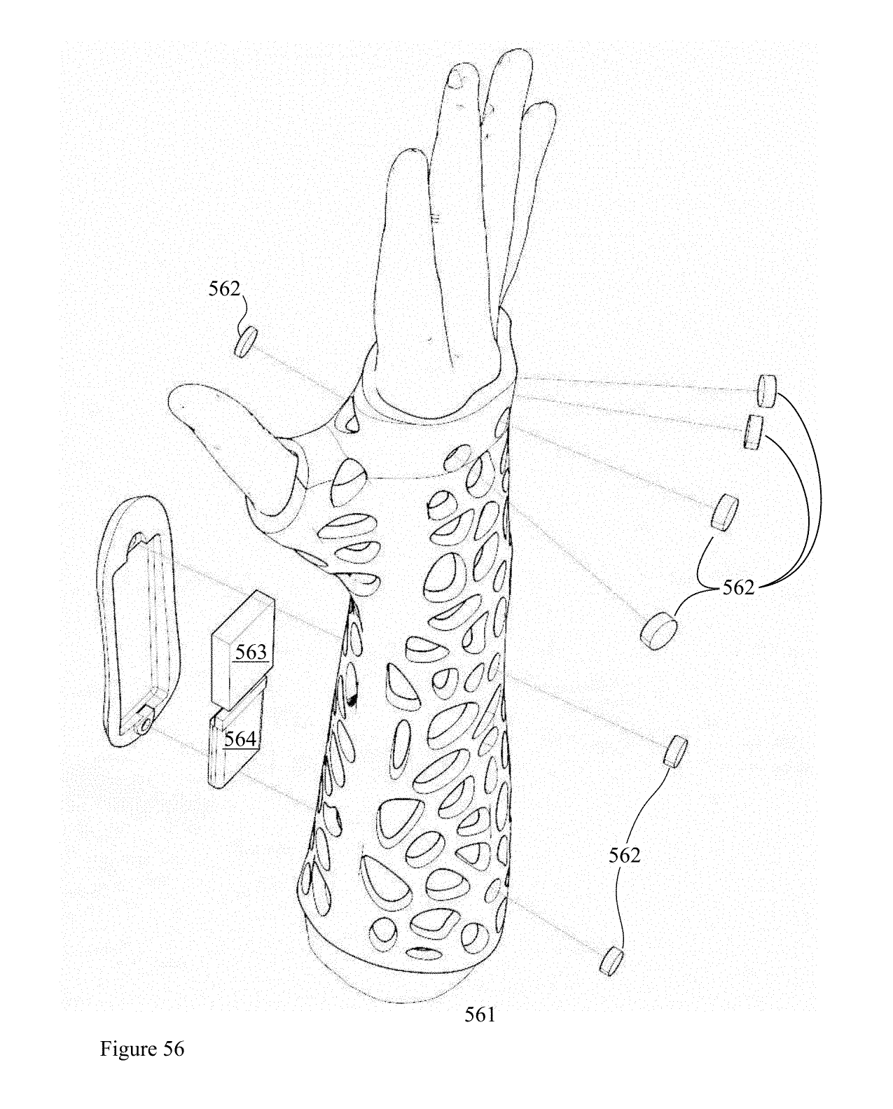

The entire personal splint can be manufactured with three-dimensional printing with flexible (skin-like) viscoelastic material or casted in a three-dimensional printed mold. FIG. 56 illustrates such splint (561) incorporating accelerometers, gyroscopes and other indicators of three-dimensional position recognition elements (562). The position data obtained from here can be used for exercise monitoring and guiding physical exercises. The system also incorporates a microcomputer with relevant capabilities (563), batteries (564), and wiring to perform the task.

In additional embodiments, the static orthotic structures and other body scaffolds of the present invention can be manufactured to provide adjustable pressure to the anatomic structures underneath. The required pressure can be generated by basic mechanical structures embedded in the device or by modifying the damping geometries with in static orthosis. Such systems allow further data collection and analysis by digitizing the adjustable force for biomechanical and orthopedic analysis. The analysis of the forces involved in progressive orthosis can provide further understanding in spasticity and fracture related cases.

Another use of an adjustable pressure monitoring system in treating and monitoring bone fractures. Typically, fractures require some level of external pressure in order to support support to the injured area. This pressure helps stabilizing the area and also help the fracture to heal. Bones are piezoelectric structures in nature, and the transfer of ions is an important contributor to fracture healing. Conventionally, the external pressure is applied by a medical professional during casting of the splint and particularly during cast's solidification process. This pressure can be applied to the fractured anatomic location by the medical professional in the same manner Due to the nature of conventional applications there is no way of measuring pressure or precisely defining the area for applying the pressure to the relevant area.

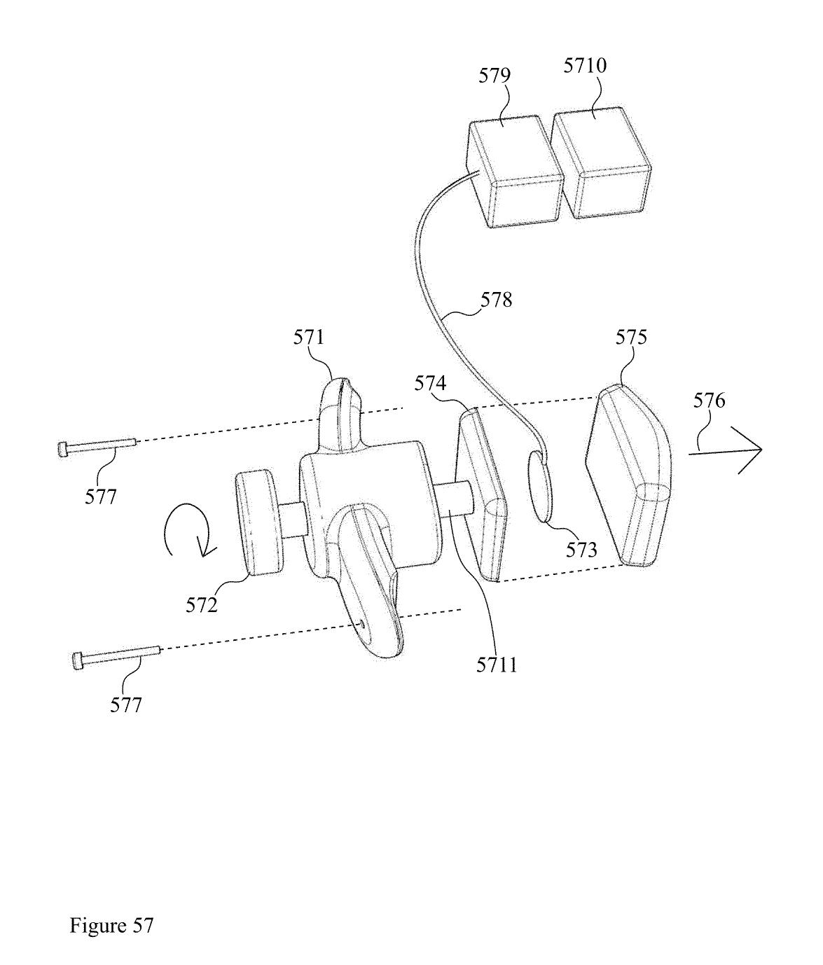

The splints and body scaffolds of the present invention can external pressure units (embedded or modular) for applying adjustable pressure to the patient anatomy to enable progressive orthotic rehabilitation. As shown in FIG. 57, a main body of a pressure delivering unit (571) may be attached to a body scaffold with screws (577) or other suitable magnetic or mechanical attachments. An intended pressure (576) is delivered through a viscoselastic body (575). The desired three-dimensional geometry of interface with the patient may be determined from data collected during the anatomic reference stage, and the structure is manufactured accordingly. The applied pressure (576) may be monitored with a pressure sensor (573) located between two units of the adjustable pressure system (574) and (575). Shaft (5711) may be a screw-type driver configured to delivering the adjustable pressure. Knob (572) may be rotated to adjust rotate the screw and adjust the pressure. The system also incorporates a microcomputer with relevant capabilities (579), batteries (5710), and wiring (5780).

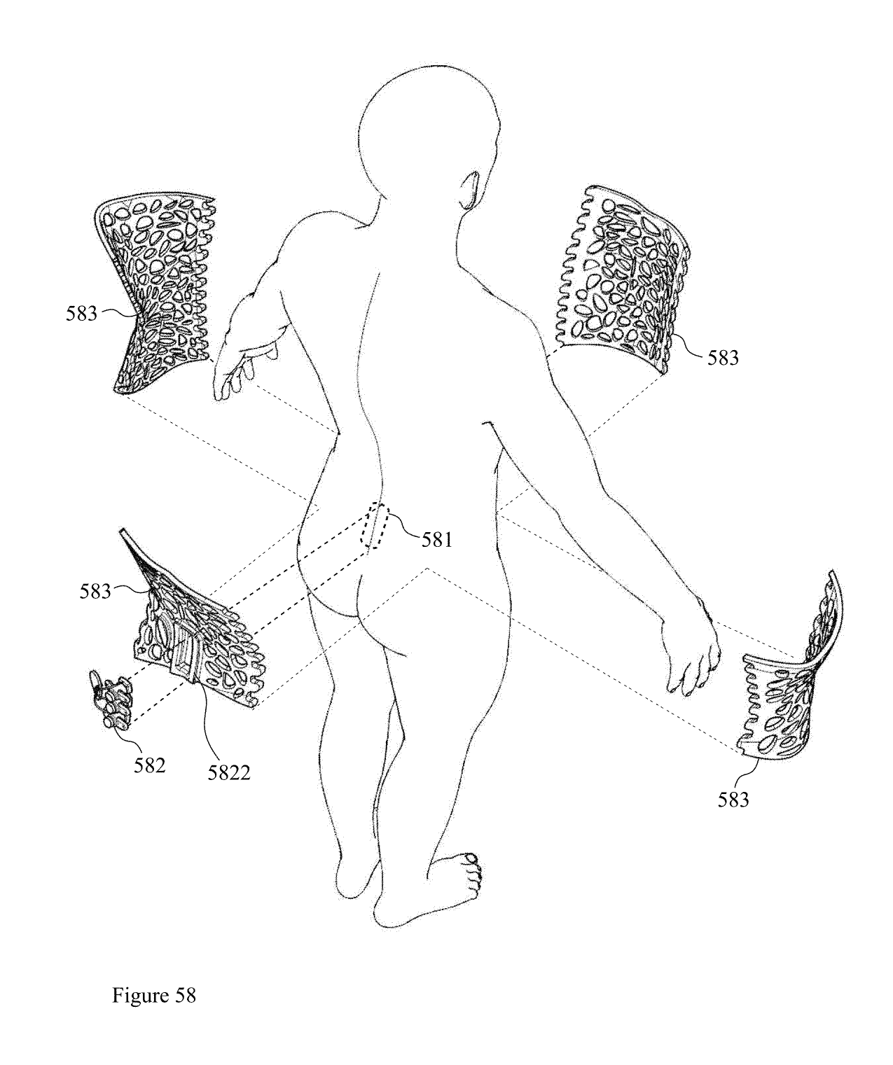

As shown in FIG. 58, a spinal orthotic (body scaffold) may incorporate four segments (583) that can be assembled to circumscribe the patient's P abdomen and/or chest. Pressure may be applied to a target zone (581) in the posterior cervical, thoracic, or lumbar region by locating a sensory unit (582) in a receptacle (5822) in the rear segment of the scaffold.

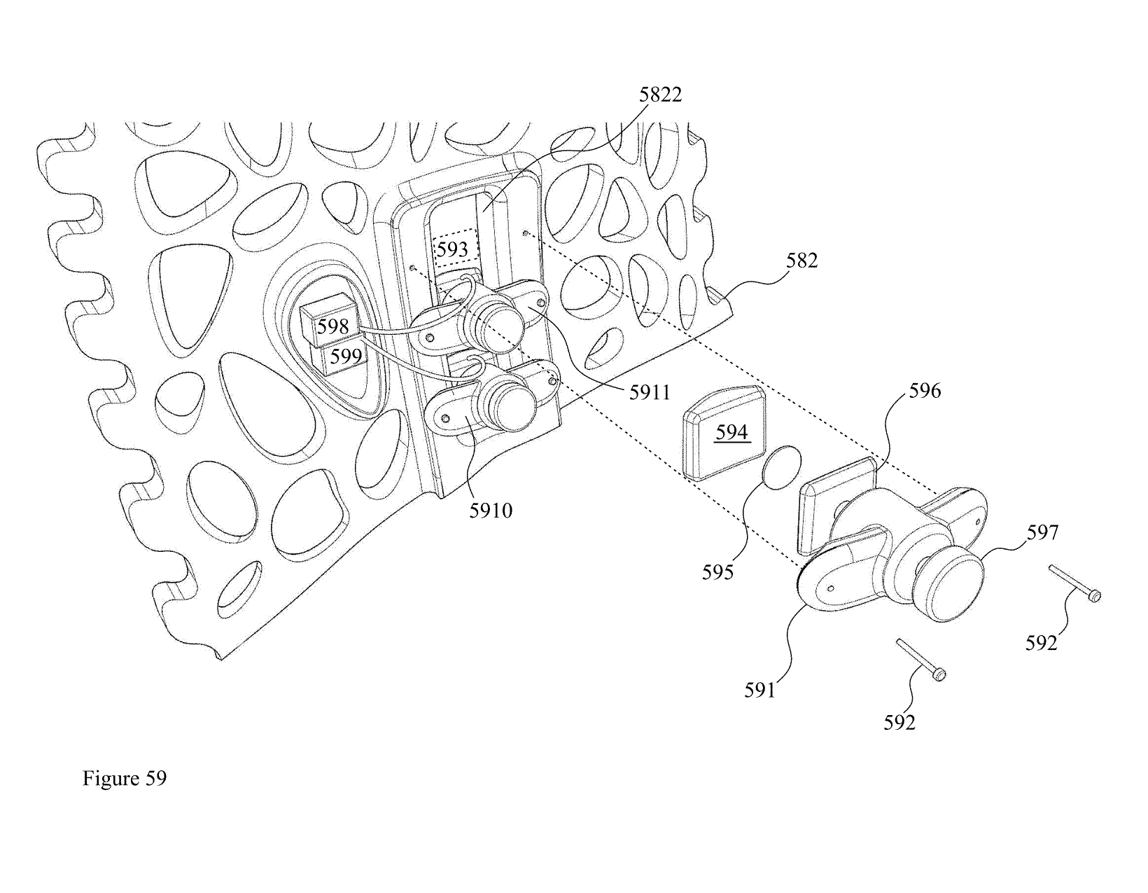

The sensory unit (582) is shown in greater detail in FIG. 59. A main body (591) is received in the receptacle 5822 and is held in place by screws (592). The anatomic target is shown at (593) on posterior lumbar spine. A pressure sensor (595) is located between a viscoelastic pad (594) and the pressure-applying member (596) of the main body (591), and a knob (597) may be rotated to advance and retract the pad (594) in an anterior-posterior direction to adjust the pressure applied to the target (593). The applied pressure is sensed and may be collected and transmitted. The system also incorporates a microcomputer with relevant capabilities (598), batteries (598), and wiring. Units 5910 and 5911 are other adjustable pressure delivering and monitoring units stabilized on the personal three-dimensional printed orthotic. Pressures may alternatively be applied by modifying the geometry of the paddings to increase or decrease the pressure transferred to the target.

The systems of the present invention may support therapeutically beneficial technologies to support and promote recovery in numerous neuromuscular and musculoskeletal conditions. Although the benefits of these technologies are known, they are difficult or impossible to administer during orthotic intervention due to physical restrictions and production methods used for manufacturing such orthotic equipment. Traditional manufacturing technologies used are unable to incorporate precision manufacturing solutions required for practical application of such technologies and methods. Current solutions also include physically tooling the orthotic in order to have access to the relevant areas (opening a window in the structure). This method is undesired by patients as the tooling process involves risk of damaging the tissue beneath and far from practical. In particular, the present invention can be used to deliver proven and other interventions that would normally be precluded by the presence of a splint. Both static and dynamic orthotic devices are convenient hubs for locating sensors and therapeutic elements delivering therapies. The therapeutic system focuses on a range of medical techniques and technologies in the areas of therapeutic and pharmaceutical assistance and enable their usage during orthotic intervention periods. The therapeutic system provides engineering solutions and related anatomic locations for; LIPUS (low pulsed ultrasound therapy), TENS (transcutaneous electrical nerve stimulation), EMS (electrical muscle stimulation), thermotherapy (heat therapy) and cryotherapy (cold therapy), LLLT (low level laser therapy), Electromagnetic therapy, massage/vibration therapy, and techniques of delivering pharmaceuticals to personal three-dimensional printed orthotics (static or dynamic) The therapeutic system relays heavily on deep understanding of therapeutic and pharmaceutical assistance technologies and methods of administration. In every case, therapeutic stimulation or delivery of medical beneficiaries will require modular or embedded probes (effectors) and equipment strategically positioned to deliver their influence to the anatomic systems (363, 364). Any temporary stabilization mechanism can be utilized for the task, mechanic (slots, screws, hinges, etc.), magnetic or chemical base.

There are many therapeutic and beneficiary technologies stimulating and improving patient anatomy with a wide range of energy transfer (acoustic, vibration, photon, electrical, electromagnetic, heat etc.) and use of pharmaceuticals. From engineering point the process will require physical modifications on personal three-dimensional printed orthotics and involved objects to provide relevant structures for integration and applications. The integration challenges can be grouped in accordance with types of administration and practicality. Therapeutic technology integration. This section covers medical therapy and beneficiary with energy transfer with modular probes and effectors. The system is built with a few basic components, a power source (mostly electrical), wiring to transfer the power coming from the power source, probes for converting (or manipulating) the power of origin to therapeutic energy Finally the probes are stabilized (modular or embedded) to the personal three-dimensional printed orthotics with various connectors (mechanical, electromagnetic, or chemical) to deliver therapy. A few examples of the delivery challenges; LIPUS (low pulsed ultrasound stimulation); Ultrasound is widely used for imaging purposes and as an adjunct to other therapies. Low-intensity pulsed ultrasound (LIPUS), having removed the thermal component found at higher intensities, is used to improve bone healing. However, its potential role in soft-tissue healing is still under investigation. The acoustic energy generated from ultrasound is produced from a piezoelectric crystal within a transducer (probe), which emits high-frequency acoustic pressure waves on the skin in direct location of the fractured area. EMS (Electrical muscle stimulation); is the elicitation of muscle contraction using electric impulses. EMS is used as a strength training tool for healthy subjects and athletes, a rehabilitation and preventive tool for partially or totally immobilized patients, a testing tool for evaluating the neural and/or muscular function in vivo, and a post-exercise recovery tool for athletes. The impulses are generated by a device and delivered through electrodes (probes) on the skin in direct proximity to the muscles on muscle masses of related muscles. Muscle masses of the extensor and fixator muscles are marked as 231, 251, 271, and 291 in FIGS. 23, 25, 27, 29. TENS (Transcutaneous Electrical Nerve Stimulation); is a therapy that uses low-voltage electrical current for pain relief. The electrodes are often placed on the area of pain or at a pressure point, creating a circuit of electrical impulses that travels along nerve fibers, acupuncture points are very convenient locations for this application. Low-level laser therapy (LLLT) is a form of laser medicine used in physical therapy and veterinary treatment that uses low-level (low-power) lasers or light-emitting diodes to alter cellular function.

In a typical application relevant probes are positioned on nerve endings, acupuncture points and joints depending on the dose, wavelength, timing, pulsing and duration. Thermotherapy; is the use of heat in therapy, such as for pain relief and health. It can take the form of a hot cloth, hot water, ultrasound, heating pad, hydrocollator packs, whirlpool baths, cordless FIR heat therapy wraps, and others. It can be beneficial to those with arthritis and stiff muscles and injuries to the deep tissue of the skin. Heat may be an effective self-care treatment for conditions like rheumatoid arthritis. Specific cryotherapy is the local or general use of low temperatures in medical therapy. Cryotherapy is used to treat a variety of benign and malignant tissue damage. Its goal is to decrease cell growth and reproduction (cellular metabolism), increase cellular survival, decrease inflammation, decrease pain and spasm, promote the constriction of blood vessels (vasoconstriction), and when using extreme temperatures, to destroy cells by crystallizing the cytosol, which is the liquid found inside cells, also known as intracellular fluid (ICF). Typically heat is generated by converting electrify in to thermal energy through electrically resistance components, such resistance components include metal heating elements, ceramic heating elements and composite heating elements. Cold is traditionally more difficult to generated by fans and complex machinery (moving hear from one location to another in controlled volumes (fridges)). In a more practical manner heat and cold can be generated with thermoelectric effects and materials. In a typical case a thermoelectric probe is placed on patient anatomy to deliver its influence. In a typical application relevant probes are positioned on injured areas, nerve endings, acupuncture points and joints depending on the case. The therapeutic system uses personal three-dimensional printed orthotic structures as a hub for therapeutic technologies concerning energy transfer.

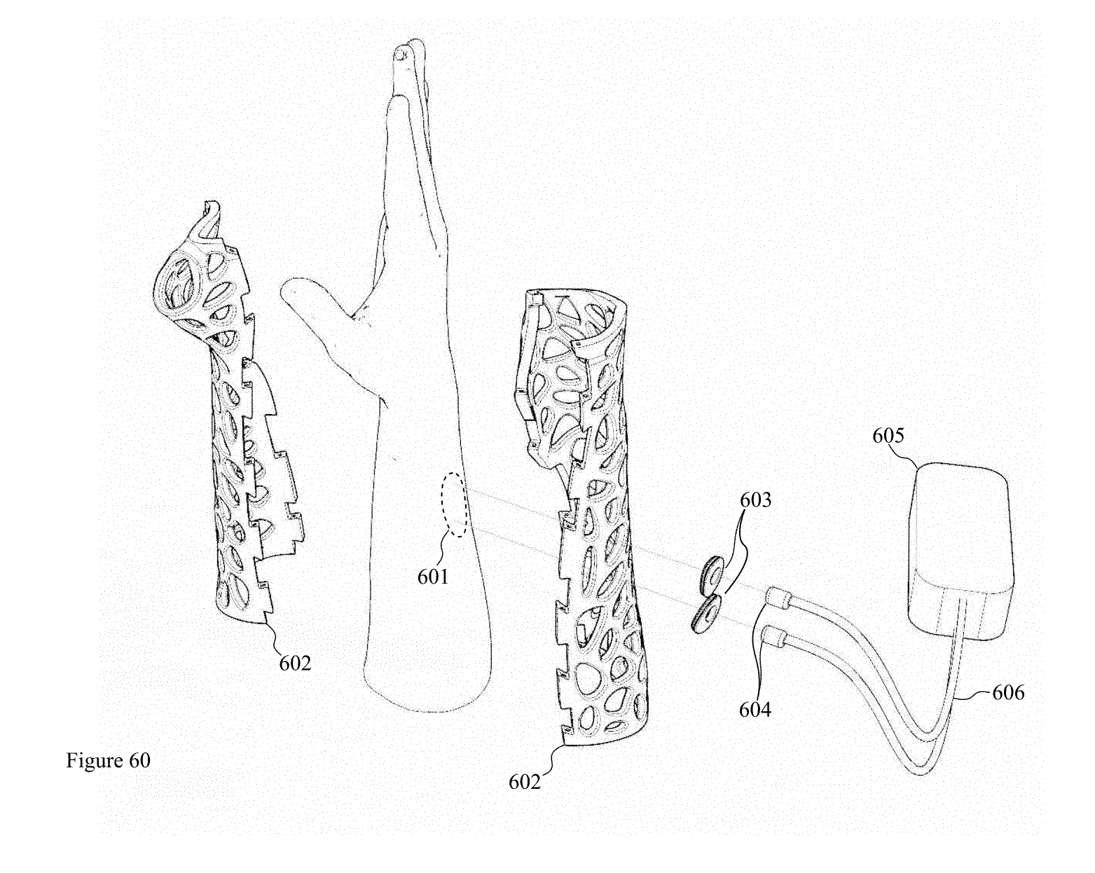

The therapeutic system uses the personal three-dimensional printed orthotic structure as a hub for therapeutic technology, for this section all the therapeutics involved are delivering their influence with dedicated probes to deliver energy to patients anatomy. Placement of probes are specific to each developing therapeutic challenge and are case sensitive. Anatomic motions discussed above can be directed to specific muscle groups according for EMS. In a typical case (FIG. 60) the desired anatomic location (601) to deliver the therapy is determined by the medical professional. A topology of the design (602) is modified to allow access to the desired anatomic region. A mechanical solution (603) is presented to physically stabilize relevant probes (604) (See also FIG. 36, 364, 363). The therapeutic device is power by a power source (605) and the energy directed with wires (606).

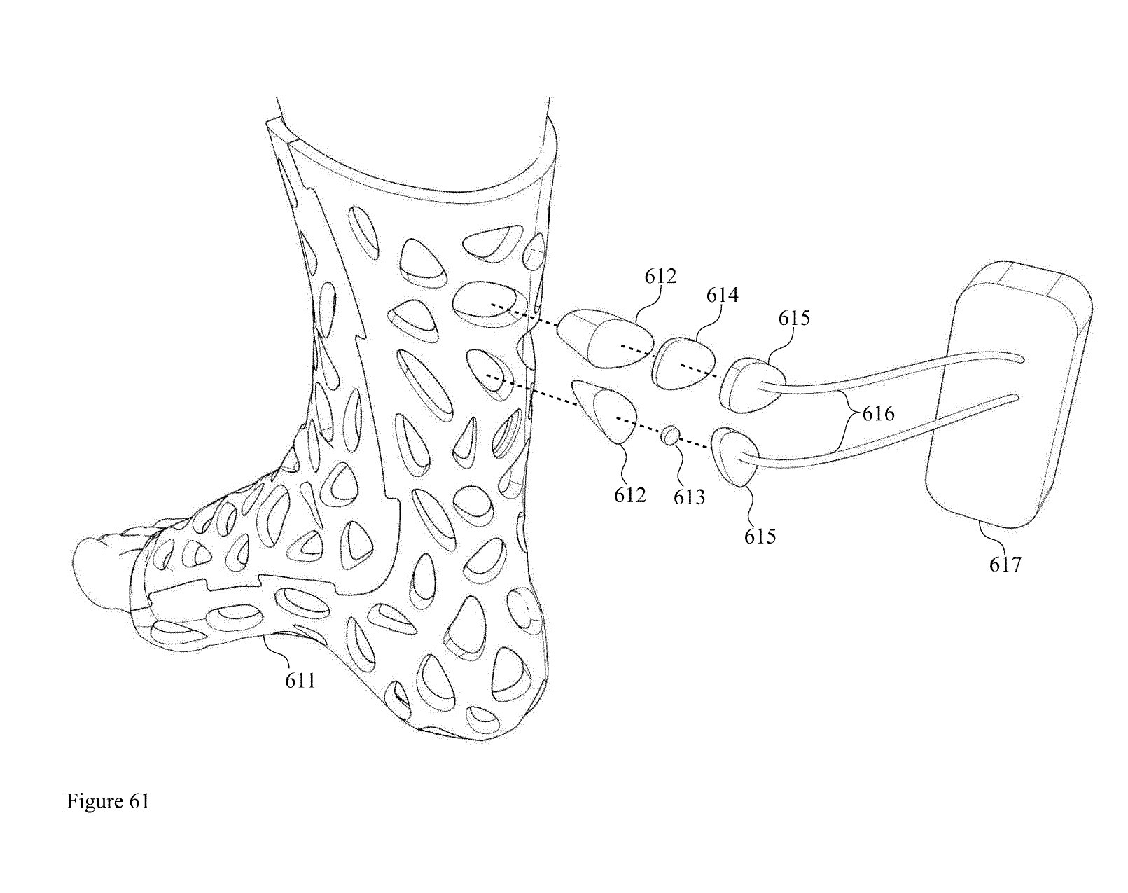

A second example is illustrated in FIG. 61 and is a lover extremity case with thermotherapy and/or cryotherapy with the use of thermoelectric and a custom heating element. Referring to FIG. 61, units providing the heat or cold are placed on a three-dimensional printed splint (611). Elements (612) provide a conformable interface to improve contact area with the skin and deliver the therapy from active heat or cold generating effectors materials. Effectors can be standard components (613) or custom build components (614). Housings (615) connect connected with wires (616) to an electrical source (617).

Both thermotherapy and cryotherapy require relatively larger areas in order to efficiently transfer energy this application is an alternative system for delivering heat or cold to a patient anatomy. Rather than placing effectors on patient anatomy this method involves circulating heated or cooled liquids trough personal three dimensional printed orthotics in order to affect larger areas. This liquid radiating method is applicable to any personal three-dimensional printed orthosis. With this method any desired/relevant area of a splint can be modified to function as an effector and deliver thermotherapy are cryotherapy to patient anatomy. From design point, additional subtractions and modifications are needed in the splint geometry to allow liquid to pass. More complex radiator geometries (paths) can also be designed with this method.

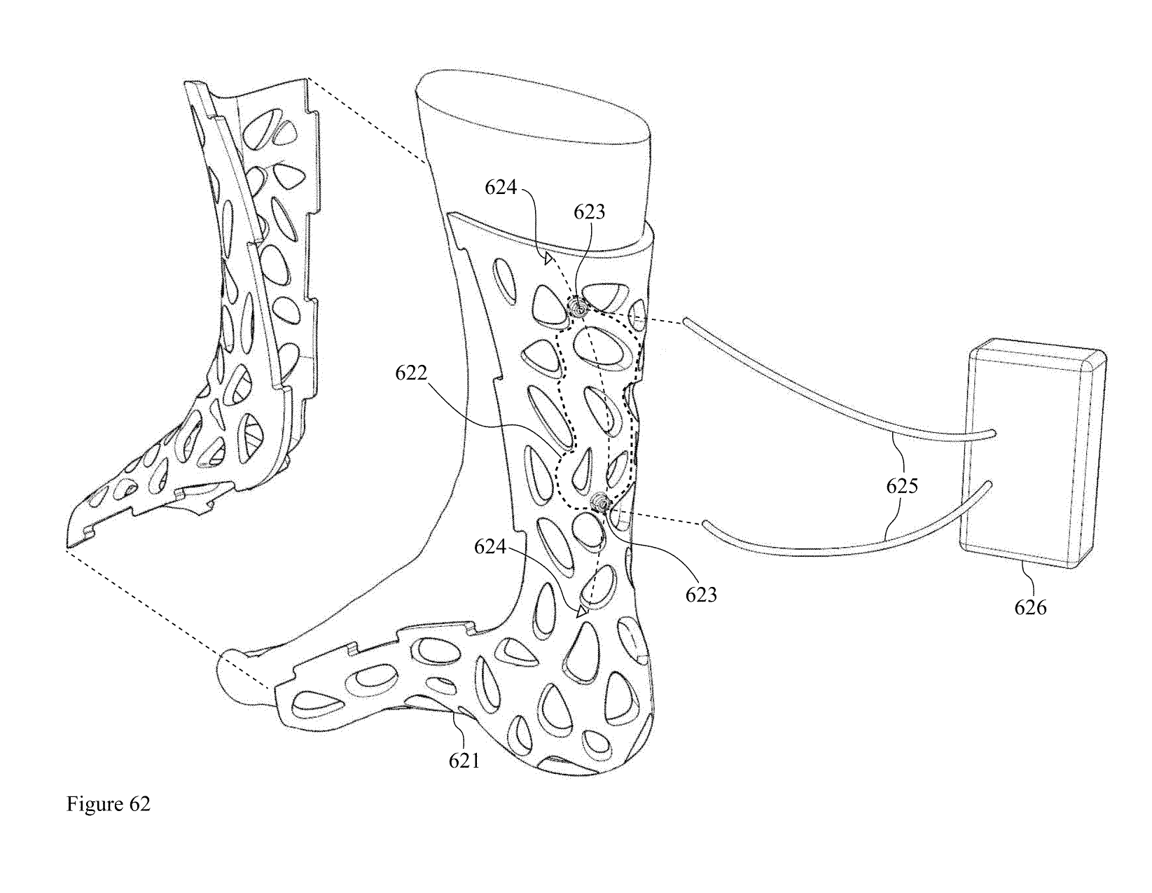

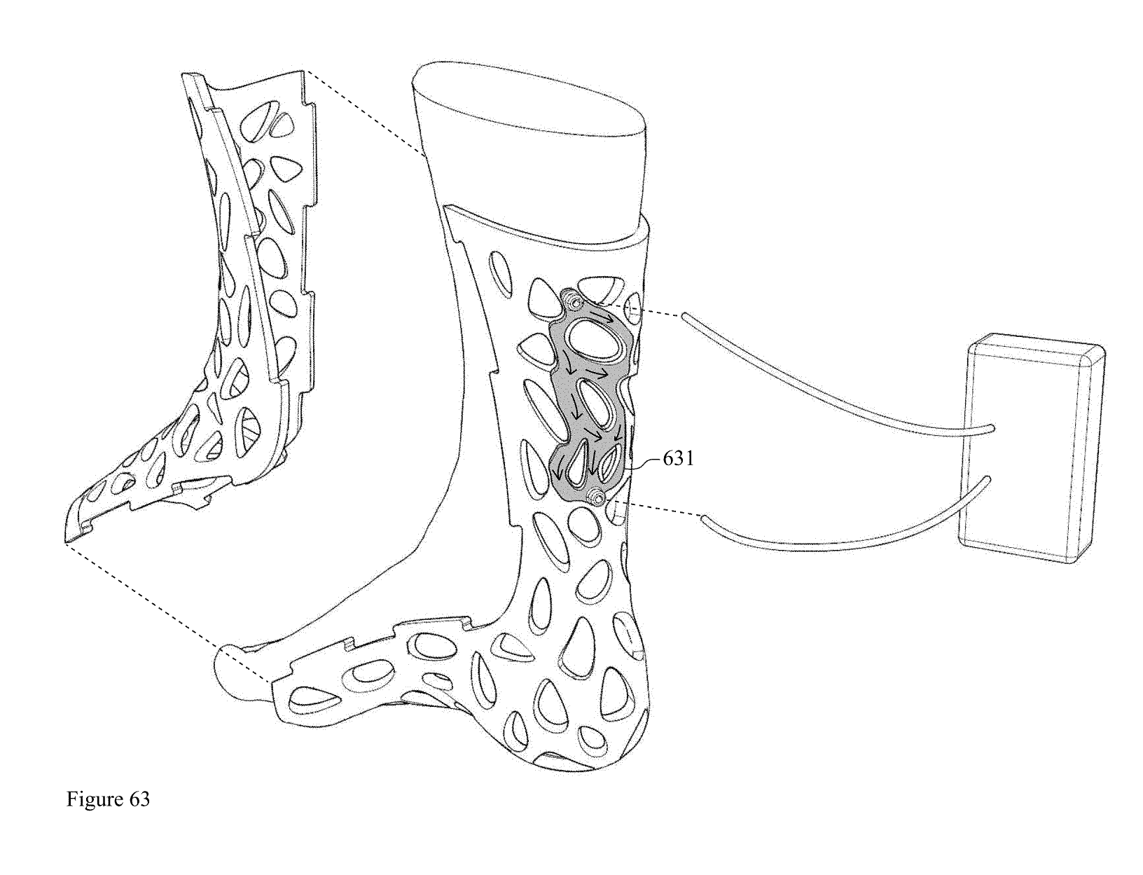

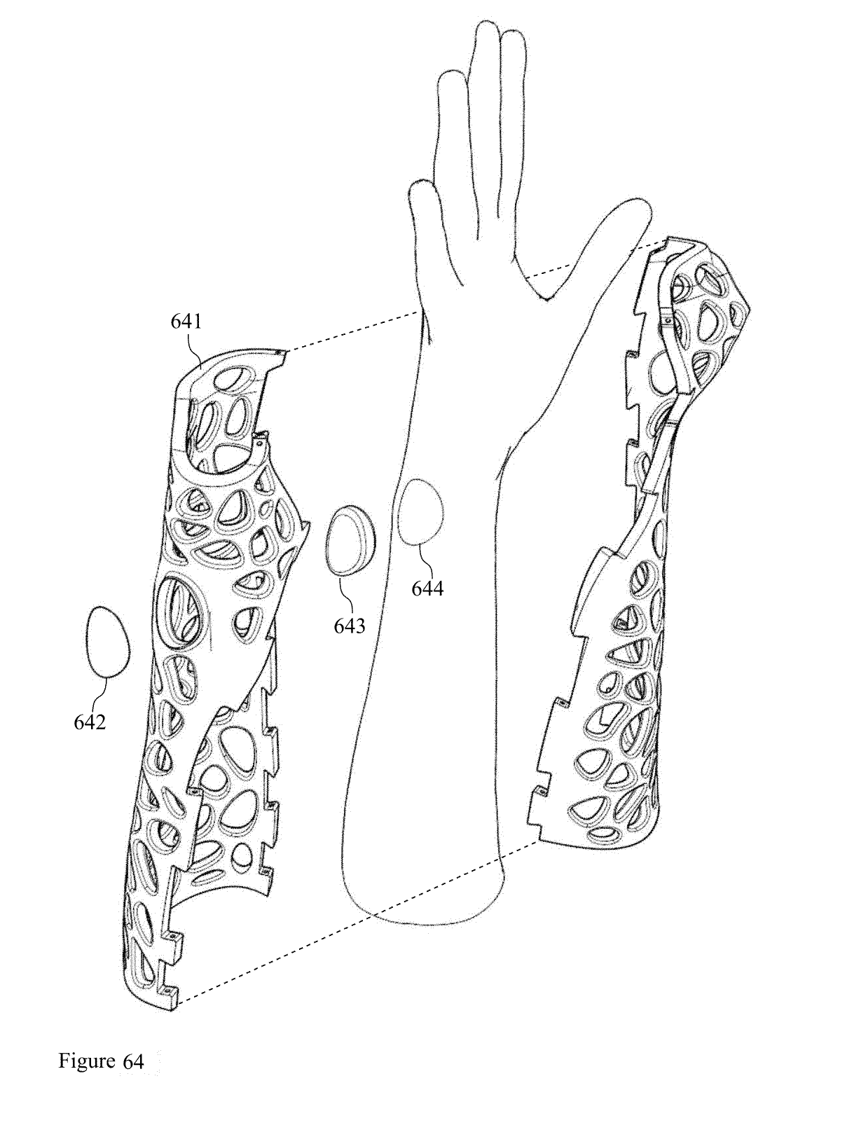

Referring to FIG. 62, another heat exchange system circulates heated or cooled liquids to achieve heat transfer. This liquid radiating method is applicable to any personal three-dimensional printed orthosis (621). Any desired area (622) of the splint can function as the effector area of the thermotherapy/cryotherapy delivering system. From design point, additional passages may be provided in the splint geometry to provide liquid flow paths. More complex radiator geometries (paths) can also be designed with this method. At least two openings and fixtures (623) and (623) are needed at the ends of the radiator structure to allow circulation. Referring to FIG. 63, a circulation path is formed at (631). Also structural effects of subscribing compartments within the splint must be taken in to consideration for durability. 624 is the section of the embedded radiator geometry. Areas marked with (641) in FIG. 64 are full body areas of the three-dimensional printed splint, areas marked with (642) are subtractions from the splint geometry to allow liquid circulation. The liquid is transferred with pipes (625) (silicone or rubber is mostly used in similar medical applications). The source of the system (626) is a circulation pump with heating and cooling components, heat adjustment settings. Combinations of different therapy methods present even greater therapeutic opportunities. Scheduling for different therapy with modular probes allows multiple therapeutic technology integration. Also placing modular or embedded effectors into to proximity of each other allows simultaneous therapeutic integration to the anatomy.

There are two possible routes for administrating pharmaceuticals trough orthotic structures to patient anatomy, dermal route or injection route. Dermal route of pharmaceutical administration is a technique of drug delivery where topical medication is the chosen method. Many topical medications are epicutaneous, meaning that they are applied directly to the skin to treat ailments via a large range of classes including but not limited to lotions, creams, ointments, liniments, liposomes, powders, pastes, films, gels, hydrogels, DMSOs (Dimethyl sulfoxide), artificial vesicles, jet injectors, dermal patches, transdermal patches, transdermal sprays, iontophoresis, non-cavitational ultrasound, cavitational ultrasound, electroporation, microneedles, thermal ablation and microdermabrasion. Injection or infusion route of pharmaceutical administration is a technique of drug delivery. Injection or infusion is simply putting fluid into the body, usually with a syringe and a hollow needle which is pierced through the skin to a sufficient depth for the material to be administered into the body. There are several methods of Injection or infusion including but not limited to, intradermal, subcutaneous (SC), intramuscular (IM), intravenous (IV), intraosseous (IO), intraperitoneal (IP), intrathecal, epidural, intracardiac, intraarticular, intracavernous, and intravitreal. The therapeutic system uses personal three-dimensional printed orthotic structures as a hub for placing pharmaceutical administration equipment and systems.

Dermal route of pharmaceutical administration require direct skin contact of relevant epicutaneous materials. Typically absorption of these materials by the skin is a process requiring time also frequent exposure to epicutaneous materials in a stable deserted environment. FIG. 64 illustrates a modified personal three-dimensional printed orthotic structure (641) with enough access to an anatomic location marked with (644). In some cases depending on the viscosity of the used substances additional sponge like container structures marked as (643) with direct skin contact can provide extended periods of absorption. Also additional structures can be beneficial for physically isolating related compartments (642).

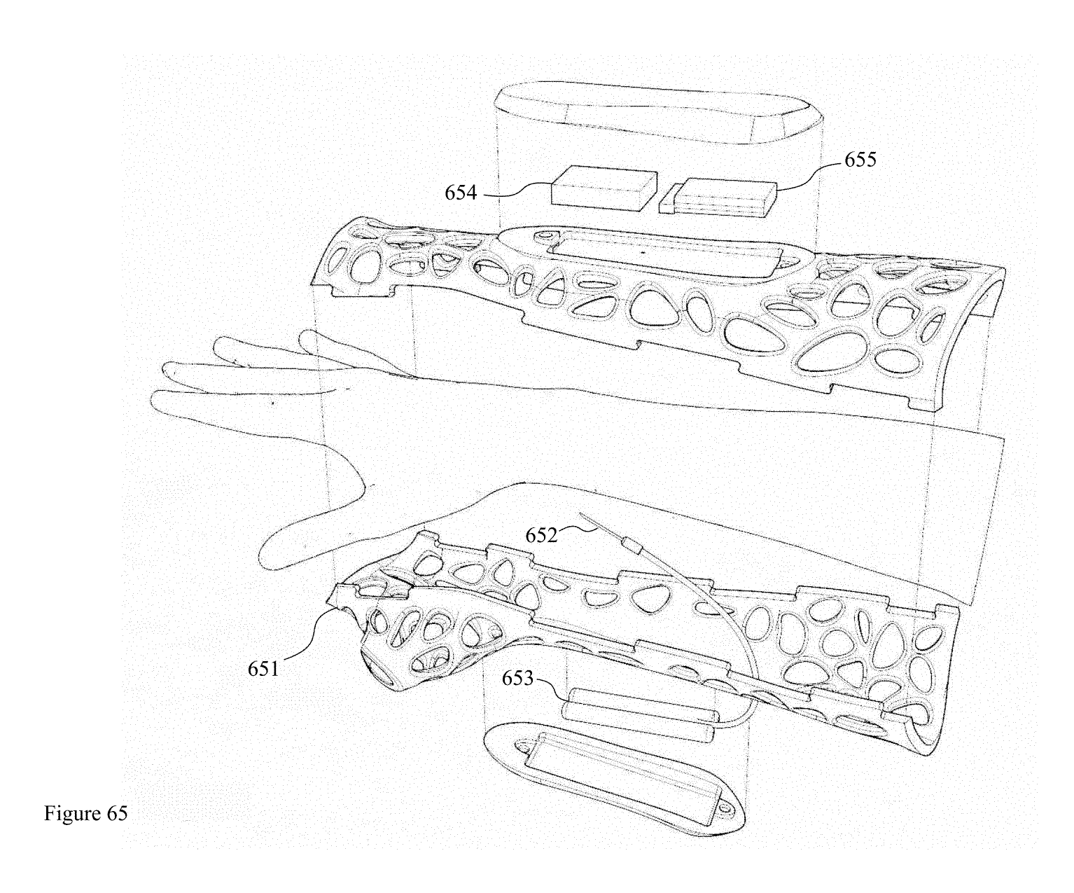

Injection and infusion of pharmaceutical substances is the final solution of the disclosure. In cases with infusion and injection the pharmaceutical fluid have to be injected via, either a pre-positioned root or a dynamic system with relevant mechanisms (spring, pressure, magnetic based) to open the necessary root of administration, to relevant anatomic structures (intravenous, intradermal, subcutaneous, and intramuscular). Mechanical adjustments in the dynamic injection system can determine the penetration level and the angle of the root of administration. FIG. 65 illustrates a modified personal three-dimensional printed orthotic structure (651) with dedicated equipment. The system incorporates a pre-installed needle (652), alternatively a mechanism with springs, magnets and air pressure can be adapted for opening the root of administration. A compartment for containing pharmaceutical liquid is demonstrated in (653) additional components of the system such as a microcomputer with relevant capabilities batteries are marked with (654) and (655). With the new generation of pharmaceutical intervention splints the users will be able to benefit from pharmaceutical effects on demand.

What is claimed is:

1. A method for fabricating a conformable body interface which can sense motion of a body joint, said method comprising:
fabricating a body scaffold which can be removably placed over a three-dimensional body surface to conform to one or more target regions of said body surface adjacent to the body joint; and
attaching at least one sensor element to the body scaffold at a location selected to position the sensor near the target region on the body surface when the body scaffold is placed over the three-dimensional body surface, wherein the sensor is configured to detect motion of the body joint.

2. A method as in claim 1, wherein the sensors is configured to detect at least one of flexion, extension, rotation, pronation, and supination.

3. A method as in claim 2, wherein the sensor is selected from the group consisting of pressure sensors, strain sensors, force sensors, accelerometers, gyroscopes, velocity sensors, tilt sensors and pulse sensors.

4. A method as in claim 1, wherein fabricating the body scaffold comprises:
obtaining a data set representing the three-dimensional, soft tissue body surface adjacent to a body joint;
wherein the data set is obtained by directly or indirectly scanning the three-dimensional soft-tissue body surface of a patient to produce an initial data set representing the geometry of the one or more target regions on the soft tissue body surface; and
modifying the initial data set to include locations for attaching the one or more sensors to the body scaffold to produce a modified data set.

5. A method as in claim 4, wherein fabricating comprises three dimensional printing based on the modified data set.

6. A method as in claim 4, wherein fabricating comprises numerically controlled machining of a substrate based on the modified data set.

7. A method as in claim 4, wherein attaching comprises inserting sensor elements into receptacles that are defined in the data set.

8. A method as in claim 4, wherein attached comprises securing the interface element to marked locations that are defined in the initial data set.

9. A method as in claim 1, wherein the sensor is positioned to detect incipient anatomic motion.

10. A method as in claim 9, wherein the sensor is placed to detect incipient flexion, extension, deviation, rotation, pronation, and supination.

11. A method as in claim 9, wherein the sensor is positioned to detect incipient anatomic motion in any one of a wrist joint, an elbow joint, an ankle joint, a toe, a spine, and a neck.

12. A method as in claim 4, wherein the initial data set defines a lattice structure which at least partially circumscribes the soft tissue surface.

13. A method as in claim 12, wherein the soft tissue surface comprises one of an upper limb, a lower limb, a wrist, an ankle, a spine, and a neck.

14. A method for generating a data set for fabricating a conformable body scaffold, said method comprising:
directly or indirectly scanning a three-dimensional soft-tissue body surface of a patient to produce an initial data set representing the surface geometry of at least one target region on the soft tissue body surface adjacent to a body joint; and
modifying the initial data set to include one or more locations for attaching one or more sensors configured to detect motion of the body joint to the conformable body scaffold to produce a final data set suitable for controlling a fabrication machine to produce the conformable body scaffold.

15. A method as in claim 14, wherein the initial data set defines a lattice structure which at least partially circumscribes the soft tissue surface.

16. A method as in claim 14, wherein the soft tissue surface comprises one of an upper limb, a lower limb, a wrist, an ankle, a spine, and a neck.

17. A conformable body interface comprising:
a body scaffold comprising a three-dimensional lattice configured to be removably placed over a three-dimensional soft-tissue surface; and
one or more sensors attached to one or more locations on the body scaffold, wherein the one or more locations selected to position the sensor near the target region on the body surface when the body scaffold is placed over the three-dimensional body surface, wherein the sensor is configured to detect motion of the body joint.

18. A conformable body interface as in claim 17, wherein the sensors are configured to detect at least one of flexion, extension, rotation, pronation, and supination.

19. A conformable body interface as in claim 17, wherein the sensor is selected from the group consisting of pressure sensors, strain sensors, force sensors, accelerometers, gyroscopes, velocity sensors, tilt sensors and pulse sensors.

20. A conformable body interface as in claim 17, wherein the body scaffold comprises an orthotic aid.

21. A conformable body interface as in claim 17, wherein the interface element further comprises a therapeutic element selected from the group consisting of an ultrasound transducer, a heat source, a cooling source, an electrical source for muscle stimulation, an electrical source for electroconvulsive therapy, or a magnetic source.
